(19) United States
(12) Patent Application Publication
(10) Pub. No.: US 2026/0016471 A1
(43) Pub. Date: Jan. 15, 2026

(54) IMPROVED ASSAY COMPOSITIONS AND METHODS

(71) Applicant: BECKMAN COULTER, INC., Brea, CA (US)

(72) Inventors: Corey M. Carlson, Minneapolis (MN); Mark D. Holland, Chaska, MN (US); Christopher R. Knutson, Chanhassen, MN (US); Michael T. Kjome, Chaska, MN (US); Miklos Szabo, Chaska, MN (US); Lauren Wolfe, Chaska, MN (US)

(21) Appl. No.: 18/875,238

(22) PCT Filed: Jul. 5, 2023
(86) PCT No.: PCT/US2023/069667

§ 371 Date: Dec. 16, 2024

Related U.S. Application Data
(60) Provisional application No. 63/358,514, filed on Jul. 5, 2022.
(60) Provisional application No. 63/389,136, filed on Jul. 14, 2022.

(51) Int. Cl. G01N 33/543; G01N 21/76; G01N 33/535; G01N 33/76

(52) U.S. Cl. CPC G01N 33/54326; G01N 21/76; G01N 33/535; G01N 33/76; G01N 2333/471; G01N 2333/59; G01N 2333/916

(57) ABSTRACT

This disclosure provides improved assays and assay methods that include compositions comprising enzymes, such as ALP, conjugated to an antigen or antibody, as well as compositions that include enzyme substrates, wherein the compositions are optimized and adapted to provide improved assay performance. The disclosure also describes compositions of matter and kits that comprise the compositions for use in immunoassays.

### CROSS-REFERENCE TO RELATED APPLICATIONS

The application is related to, and claims the benefit of priority of, U.S. Provisional Patent Application Ser. No. 63/358,514, filed on Jul. 5, 2022, and U.S. Provisional Patent Application Ser. No. 63/389,136, filed on Jul. 14, 2022, each of which is incorporated herein by reference in its entirety.

### BACKGROUND

Immunoassays are critical tools in many areas of medicine and research and discovery, including disease diagnosis, disease prognosis, disease risk assessment, determining and monitoring therapeutic treatment and efficacy, as well as clinical pharmacokinetic and bioequivalence studies in drug discovery and pharmaceutical industries. Continuing to improve assay specificity, throughput, sensitivity, and increase the number of validated target analytes will provide better clinical evaluation, therapeutic interventions.

Alkaline phosphatase (ALP) is an enzyme used in combination with a colorimetric or chemiluminescent reagent in immunoassays. Typically, upon catalysis of a substrate by ALP, chemiluminescent or colored fluorescent signals that can be measured and quantified are produced. Enzymes, such as ALP, and associated substrate molecules as well as compositions comprising the same that are used in an assay can be result-effective variables for overall assay performance. Thus, it is of significant benefit to develop enzyme conjugates or derivatives, substrate compounds and compositions comprising the same that provide for improved assay performances (e.g., increased activity, stability, analyte signal, and/or reduced background signal, lower limits of detection and quantitation, improved signal-to-noise, and allows for the use of lower concentrations and/or amounts of assay reagents.).

### SUMMARY

In an aspect, the disclosure relates to a container comprising:
a. at least one reagent comprising particles, wherein a mass of the particles is at least 0.1 μg and up to 100 μg; and
b. a chemiluminescent substrate formulation wherein the chemiluminescent substrate formulation comprises a chemiluminescent compound, or salt thereof, of formula I:

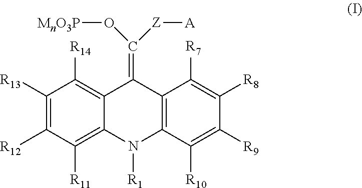

wherein
A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
R₁ is selected from the group consisting of C₅₋₁₄aryl, C₁₋₆ alkyl, C₁₋₆ haloalkyl, and C₅₋₁₄ aralkyl groups;
R₇-R₁₄ are independently H, C₁₋₆ alkoxy, halo, C₁₋₄alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
R₁₅ is C₁₋₆ alkyl;
each M is independently selected from the group consisting of H, an alkali metal, alkaline earth metal, transition metal, ammonium, phosphonium, organic amine salt, and an amino acid salt;
Z is O or S; and
n is 0, 1, or 2.

In some embodiments of this aspect, the container can be situated in an immunoassay analyzer or instrument. In some further embodiments, the immunoassay analyzer or instrument comprises a system that uses disposable tips remove a sample aliquot from a sample, a dispense control system, an image capture device and/or camera unit that may be used to provide feedback on an immunoassay analyzer or instrument performance in real time, a dispenser that heats a wash fluid prior to it being added to the reaction vessel, or a mechanism that is adapted to de-gas a fluid, or a combination thereof. In yet further embodiments, the immunoassay system comprises a luminometer system, the luminometer system, comprising: a photomultiplier tube configured to sense photons emitted from an assay reaction over a period of time; an analog circuit configured to provide an assay analog voltage based on the photons emitted from the assay over the period of time, the analog circuit comprising: a current sensing resistor coupled to convert current from the photomultiplier tube to a voltage; an amplifier configured to amplify the voltage; and a dedicated electrical connection between a terminal of the current sensing resistor and a terminal of the amplifier; a counter circuit configured to provide an assay photon count based on the photons emitted from the assay over the period of time; and a luminometer controller configured to: in response to the assay analog voltage being greater than a predetermined value, calculating a relative light unit value of the photons emitted from the assay over the period of time based on the assay analog voltage and an optimized linear function.

In some embodiments of this aspect, the container is adapted for use in an immunoassay method.

In another aspect, the disclosure relates to an immunoassay analyzer or instrument comprising a container, wherein the immunoassay analyzer or instrument comprises a system that uses disposable tips remove a sample aliquot from a sample, a dispense control system, an image capture device and/or camera unit that may be used to provide feedback on immunoassay analyzer or instrument performance in real time, a dispenser that heats a wash fluid prior to it being added to the reaction vessel, or a mechanism that is adapted to de-gas a fluid, and/or wherein the immunoassay system comprises a luminometer system, the luminometer system, comprising: a photomultiplier tube configured to sense photons emitted from an assay reaction over a period of time; an analog circuit configured to provide an assay analog voltage based on the photons emitted from the assay over the period of time, the analog circuit comprising: a current sensing resistor coupled to convert current from the photomultiplier tube to a voltage; an amplifier configured to amplify the voltage; and a dedicated electrical connection between a terminal of the current sensing resistor and a terminal of the amplifier; a counter circuit configured to provide an assay photon count based on the photons emitted from the assay over the period of time; and a luminometer controller configured to: in response to the assay analog voltage being greater than a predetermined value, calculating a relative light unit value of the photons emitted from the assay over the period of time based on the assay analog voltage and an optimized linear function; and wherein the container comprises up to 100 μg of paramagnetic microparticles.

In another aspect, the disclosure relates to an immunoassay analyzer or instrument comprising a container, wherein the immunoassay analyzer or instrument comprises a system that uses disposable tips remove a sample aliquot from a sample, a dispense control system, an image capture device and/or camera unit that may be used to provide feedback on analyzer or instrument performance in real time, a dispenser that heats a wash fluid prior to it being added to the reaction vessel, or a mechanism that is adapted to de-gas a fluid, and/or wherein the immunoassay system comprises a luminometer system, the luminometer system, comprising: a photomultiplier tube configured to sense photons emitted from an assay reaction over a period of time; an analog circuit configured to provide an assay analog voltage based on the photons emitted from the assay over the period of time, the analog circuit comprising: a current sensing resistor coupled to convert current from the photomultiplier tube to a voltage; an amplifier configured to amplify the voltage; and a dedicated electrical connection between a terminal of the current sensing resistor and a terminal of the amplifier; a counter circuit configured to provide an assay photon count based on the photons emitted from the assay over the period of time; and a luminometer controller configured to: in response to the assay analog voltage being greater than a predetermined value, calculating a relative light unit value of the photons emitted from the assay over the period of time based on the assay analog voltage and an optimized linear function; and wherein the container comprises a sample aliquot with a volume of 2 μl to 50 μl.

In related aspects and embodiments, the disclosure relates to the use of the immunoassay instrument in an immunoassay method.

In another aspect, the disclosure relates to a method comprising: contacting a sample that is suspected of containing a target analyte with an antibody composition, to form a mixture; incubating the mixture in a container or vessel; and adding a substrate composition to the container or vessel to form a reaction mixture; wherein the antibody composition comprises: an antibody to the target analyte conjugated to alkaline phosphatase (ALP) or an antigen conjugated to ALP; a buffer comprising a zinc salt and a magnesium salt; wherein the buffer concentration of free Zn²⁺ is in a range of 5 μM to 1 mM and the concentration of free Mg²⁺ is in a range of 150 times the concentration of free Zn²⁺ to 3500 times the concentration of free Zn²⁺, when the pH of the composition is in a range of 5.5 to 6.4; and wherein the substrate composition comprises: a chemiluminescent compound, or salt thereof, of formula I:

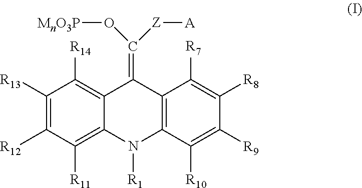

wherein
A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
R₁ is selected from the group consisting of C₅₋₁₄aryl, C₁-6 alkyl, C₁₋₆ haloalkyl, and C₅₋₁₄ aralkyl groups;
R₇-R₁₄ are independently H, C₁₋₆ alkoxy, halo, C₁₋₄alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
R₁₅ is C₁₋₆ alkyl;
each M is independently selected from the group consisting of H, an alkali metal, alkaline earth metal, transition metal, ammonium, phosphonium, organic amine salt, and an amino acid salt;
Z is O or S; and
n is 0, 1, or 2.

In another aspect, the disclosure relates to an immunoassay method comprising: in a reaction vessel, contacting a sample suspected of containing an analyte with 0.1 μg-100 μg of particles; and (a) subjecting the reaction vessel to further sample processing on an immunoassay instrument.

In another embodiment of this aspect, the disclosure relates to an immunoassay method to detect alpha-fetoprotein (AFP) in a sample, wherein the immunoassay method comprises: contacting a sample that is suspected of containing AFP with an anti-AFP antibody conjugated to an enzyme, to form a mixture, wherein the sample is a blood sample, a plasma sample, or an amniotic fluid sample; incubating the mixture in a container or vessel; adding a chemiluminescent substrate to the container or vessel to form a reaction mixture; incubating the reaction mixture; measuring light generated by the reaction mixture; and detecting a concentration of AFP in the sample from the light generated by the reaction mixture; wherein a Limit of Blank (LoB) of the immunoassay method is 0.08-0.16 ng/ml, a Limit of Detection (LoD) of the immunoassay method is 0.22-0.23 ng/mL, and/or a Limit of Quantitation (LoQ) of the immunoassay method is 0.13-0.20.

In another embodiment of this aspect, the disclosure relates to an immunoassay method to detect thyroid stimulating hormone (TSH) in a sample, wherein the immunoassay method comprises: contacting a sample that is suspected of containing TSH with an anti-TSH antibody conjugated to an enzyme, to form a mixture, wherein the sample is a plasma sample, a urine sample, an amniotic fluid sample, a whole blood sample, a synovial fluid sample, a cerebrospinal fluid sample, a saliva sample, a seminal fluid sample, a nasal fluid sample, a mucous sample, or a bronchoalveolar lavage sample; incubating the mixture in a container or vessel; adding a chemiluminescent substrate to the container or vessel to form a reaction mixture; incubating the reaction mixture; measuring light generated by the reaction mixture; and detecting a concentration of TSH in the sample from the light generated by the reaction mixture; wherein a Limit of Blank (LoB) of the immunoassay method is 0.001-0.002 uIU/mL, a Limit of Detection (LoD) of the immunoassay method is 0.002-0.003 uIU/mL, and/or a Limit of Quantitation (LoQ) of the immunoassay method is 0.0008-0.001 uIU/mL.

In another embodiment of this aspect, the disclosure relates to an immunoassay method to detect beta human chorionic gonadotropin (βhCG) in a sample, wherein the immunoassay method comprises: contacting a sample that is suspected of containing βhCG with an anti-βhCG antibody conjugated to an enzyme, to form a mixture, wherein the sample is a plasma sample, a urine sample, an amniotic fluid sample, a whole blood sample, a synovial fluid sample, a cerebrospinal fluid sample, a saliva sample, a seminal fluid sample, a nasal fluid sample, a mucous sample, or a bronchoalveolar lavage sample; incubating the mixture in a container or vessel; adding a chemiluminescent substrate to the container or vessel to form a reaction mixture; incubating the reaction mixture; measuring light generated by the reaction mixture; and detecting a concentration of βhCG in the sample from the light generated by the reaction mixture; wherein a Limit of Blank (LoB) of the immunoassay method is 0.0-0.1 mIU/mL, a Limit of Detection (LoD) of the immunoassay method is 0.2 mIU/mL, and/or a Limit of Quantitation (LoQ) of the immunoassay method is 0.1-0.2 mIU/mL.

In another embodiment of this aspect, the disclosure relates to an immunoassay method to detect free thyroxine 4 (T4) in a sample, wherein the immunoassay method comprises: contacting a sample that is suspected of containing T4 with an anti-T4 antibody conjugated to an enzyme, to form a mixture, wherein the sample is a plasma sample, a urine sample, an amniotic fluid sample, a whole blood sample, a synovial fluid sample, a cerebrospinal fluid sample, a saliva sample, a seminal fluid sample, a nasal fluid sample, a mucous sample, or a bronchoalveolar lavage sample; incubating the mixture in a container or vessel; adding a chemiluminescent substrate to the container or vessel to form a reaction mixture, wherein the chemiluminescent substrate is a substrate for the enzyme; incubating the reaction mixture; measuring light generated by the reaction mixture; and detecting a concentration of T4 in the sample from the light generated by the reaction mixture; wherein a Limit of Blank (LoB) of the immunoassay method is less than 0.18 ng/mL.

These various aspects and embodiments are not intended to describe every disclosed embodiment or every implementation of the disclosure, and the description that follows more particularly exemplifies certain illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. shows an illustration of an Allowable Total Difference (ATD) zone. Image adapted from Assay Migration guidance document (see, “Guidance for Industry and Staff: Assay Migration Studies for In Vitro Diagnostic Devices” issued 2013, available online as a regulatory guidance document for assay migration studies for in vitro diagnostic devices at the FDA's website.

FIG. 2 shows reproducibility of the Access AFP assay, as evaluated following Clinical & Laboratory Standard Institute (CLSI) guidance in EP05-A3 on both DxI 9000 and Access 2 immunoassay analyzers including between-site, between-lot, between-day, between-run, and within-run variance components. Reproducibility on the DxI 9000 immunoassay analyzer was markedly improved compared to reproducibility on the Access 2 immunoassay analyzer.

FIG. 3 shows the results of a quantitative comparison study that compared the Access AFP assay on a DxI 9000 immunoassay analyzer to the Access AFP assay on an Access 2 immunoassay analyzer. Regression analysis following CLSI EP09C-ED3 was completed in addition to comparison to Allowable Total Difference (ATD) zones prescribed within the Assay Migration guidance (see, 2013 guidance document, above).

FIG. 4 shows the results of an evaluation of Access AFP assay imprecision following CLSI EP05-A3 on a DxI 9000 immunoassay analyzer on each of three reagent lots. A representative reagent lot is shown for illustration; all lots yielded acceptable performance.

FIG. 5 shows the results of an evaluation of Access AFP assay Limit of Blank (LoB), Limit of Detection (LoD), and Limit of Quantitation (LoQ) following CLSI EP17-A2 on a DxI 9000 immunoassay analyzer on each of three reagent lots. A representative precision profile at low concentrations is shown.

FIG. 6 shows the results of an evaluation of Access AFP assay linearity following CLSI EP06-Ed2 on a DxI 9000 immunoassay analyzer across 3 reagent lots. Acceptable nonlinearity was observed for each individual assessment.

FIG. 7 shows reproducibility of the Access hsTnI assay, as evaluated following CLSI EP05-A3 on both Dx1 9000 and Access 2 immunoassay analyzer including between-site, between-lot, between-day, between-run, and within-run variance components. Independent studies were completed for both serum and lithium heparin plasma. Reproducibility on the DxI 9000 immunoassay analyzer was markedly improved compared to reproducibility on the Access 2 immunoassay analyzer.

FIG. 8A-FIG. 8B show the results of a quantitative comparison study that compared the Access hsTnI assay (lithium heparin plasma (8A); serum (8B)) on a DxI 9000 immunoassay analyzer to the Access hsTnI assay on a Access 2 immunoassay analyzer. Regression analysis following CLSI EP09C-ED3 was completed in addition to comparison to Allowable Total Difference (ATD) zones prescribed within the Assay Migration guidance. Study design criteria were met.

FIG. 9 shows the results of an evaluation of Access hsTnI assay imprecision following CLSI EP05-A3 on a DxI 9000 immunoassay analyzer on each of three reagent lots. Serum and lithium heparin plasma were evaluated individually. A representative reagent lot is shown for illustration; all lots yielded acceptable performance

FIG. 10 shows the results of an evaluation of Access hsTnI assay LoB, LoD, and LoQ following CLSI EP17-A2 on a DxI 9000 immunoassay analyzer on each of three reagent lots. Serum and lithium heparin plasma were evaluated individually. A representative precision profile at low concentrations is shown.

FIG. 11 shows the results of an evaluation of Access hsTnI assay linearity following CLSI EP06-Ed2 on a DxI 9000 immunoassay analyzer across 3 reagent lots. Independent studies were completed for serum and lithium heparin plasma. Acceptable nonlinearity was observed for each individual assessment.

FIG. 12 shows an example embodiment of APS-5, an acridan phosphate ester, included in LUMI-PHOS PRO (Lumigen, Inc., Southfield, MI), a substrate in accordance with the disclosure.

FIG. 13 shows the relative light unit (RLU) response when detected using LUMI-PHOS 530 (Lumigen, Inc., Southfield, MI) or an APS-5-containing substrate (LUMI-PHOS PRO) before (April Curve) and after (Oct Curve) storage of an ALP-containing reagent pack at 4° C. for 6 months.

FIG. 14A-FIG. 14C show the effect of altering concentrations of zinc and magnesium on the percent change in ALP activity after 3 days at 37° C. at pH 6 (FIG. 14A), pH 7 (FIG. 14B), and pH 8 (FIG. 14C).

FIG. 15 shows Mg/Zn ratio plotted versus relative light unit (RLU) loss for a cTnI assay, as further described in Example 3D.

### DETAILED DESCRIPTION

In a general sense, the disclosure provides immunoassay methods and compositions comprising assay substrates and formulations that find use in those methods, and/or systems (including, for example, instruments or analyzers) that provide for assays that have improved performance or other advantages, relative to the state of the art. In an aspect, the disclosure provides improved assays comprising an assay substrate (for example, a chemiluminescent substrate) and/or formulation comprising the same, in accordance with the example embodiments described herein. In another aspect, the disclosure provides improved assays comprising an assay formulation (e.g., an enzyme or conjugated enzyme in, e.g., a buffer formulation) in accordance with the example embodiments described herein. In yet other aspects, the disclosure provides improved assays comprising a combination of an assay substrate and at least one assay formulation in accordance with the example embodiments described herein. In further aspects, the disclosure provides kits and compositions of matter comprising a combination of an assay substrate and at least one assay formulation in accordance with the example embodiments described herein. In another aspect, the disclosure relates to a method comprising: contacting a sample that is suspected of containing a target analyte with an antibody composition, to form a mixture; incubating the mixture in a container or vessel; and adding a substrate composition to the container or vessel to form a reaction mixture; wherein the antibody composition comprises: an antibody to the target analyte conjugated to alkaline phosphatase (ALP) or an antigen conjugated to ALP; a buffer comprising a zinc salt and a magnesium salt; wherein the buffer concentration of free Zn²⁺ is in a range of 5 μM to 1 mM and the concentration of free Mg²⁺ is in a range of 150 times the concentration of free Zn²⁺ to 3500 times the concentration of free Zn²⁺, when the pH of the composition is in a range of 5.5 to 6.4; and wherein the substrate composition comprises a chemiluminescent compound as described herein. In some further aspects the disclosure provides one or more containers comprising one or more of buffers, one or more reagents comprising a solid substrate; an antibody; an antibody-enzyme conjugate; and/or a chemilumiscent compound. In some further aspects the disclosure provides one or more containers comprising a chemilumiscent compound and an enzyme in a ratio of 1 μg chemiluminescent compound to 1500 molecules of enzyme. In other aspects, the disclosure provides an immunoassay analyzer that comprises a dispense control system, an image capture device or camera unit, a dispenser or mechanism adapted to degas a fluid, and a luminometer system, and further comprising one or more of the containers in accordance with the aspects and embodiments disclosed herein.

In embodiments of the above aspects, the improvement in the assay performance can refer to any result (that is, one or more measurable parameters) of the particular assay that shows improvement relative to the assay performance in the absence of one or more of the assay substrates, formulations, methods, and/or systems in accordance with the disclosure. In some further embodiments, the improvement in the assay can comprise any one or more of improved assay precision, improved assay sensitivity, improved assay reproducibility, improved assay signal-to-noise (S/N) ratio (e.g., an improved signal, an improved reduction in assay background/noise, or both), improved assay limit of blank (LoB), improved assay limit of detection (LoD), improved assay limit of quantitation (LoQ), and/or improvement in reagent consumption (e.g., increasing the number of tests per assay reagent pack/unit). The terms LoB, LoD, and LoQ are used herein consistent with the definitions provided in CLSI EP17-A2. (See, CLSI. Evaluation of Detection Capability for Clinical Laboratory Measurement Procedures; Approved Guideline—Second Edition. CLSI document EP17-A2. Wayne, PA: Clinical and Laboratory Standards Institute; 2012.). In some further embodiments the improvement in the assay relates to an increase in assay sensitivity (i.e., the assays provide for the detection of lower amounts of a target analyte in a sample). In such embodiments, the assays can further provide for such increase in detection sensitivity without any loss in assay precision and/or reproducibility. Such improvements in assay sensitivity can provide a number of advantages over state of the art assays including detection of a disorder or disease at an earlier stage (e.g., early onset, high risk of developing a disease, or increased likelihood of developing a disease) which can provide for increased success and improvements associated with therapeutic interventions and patient outcomes. In some other embodiments, improved assay sensitivity can allow for the detection of additional disease states that are shown to be associated with lower concentrations of a target analyte concentration, and which are not detectable using the state of the art assay systems and methods. Accordingly, assays providing for increased sensitivity for one or more target analytes provides for a substantial improvement in early identification of patient classes (e.g., patients at risk of developing a disease), as well as identifying, diagnosing, and treating various conditions, disorders, and diseases associated with one or more biomarkers, such as those described herein.

In embodiments of the above aspects, the assay substrates, formulations, methods, and/or systems that can be used in methods comprising measuring concentration of one or more target analytes in a liquid sample (e.g., a biological sample obtained from a subject, such as, for example, a human patient). Exemplary sample types include but are not limited to serum, plasma, urine, amniotic fluid, whole blood, synovial fluid, cerebrospinal fluid, saliva, seminal fluid, nasal fluid, mucous (e.g., swabs, sputum, etc.) or bronchoalveolar lavage, and the like.

### Definitions

When used herein, the terms “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

The term “comprises”, “includes”, “has” and similar variations thereof do not have a limiting meaning where these terms appear in the description and claims. Such terms will be understood to imply the inclusion of a stated step or element or group of steps or elements but not the exclusion of any other step or element or group of steps or elements. In contrast, the term “consisting of” means including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

As used herein, the recitations of numerical ranges by endpoints include all numbers subsumed within that range (for example, 1 to 5 includes 1, 1.5, 2, 2.75, 3, 3.80, 4, 5, etc.). Further, reference within the disclosure of the phrase, “up to” a stated number (for example, up to 50) includes the number (for example, 50).

When used herein, the terms “in the range” or “within a range” (and similar phrases) with reference to numerical values includes the endpoints of the stated numerical range.

For any method disclosed herein that includes discrete steps, the steps may be conducted in any feasible order, and if appropriate and operationally possible, any combination of two or more steps may be conducted simultaneously.

All headings are for the convenience of the reader and should not be used to limit the meaning of the text that follows the heading, unless so specified. Further, all patent and non-patent references that are referenced herein are incorporated by reference herein in their entirety.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers that reflect quantity of components, a measured value, molecular weights, and so forth as used in the specification and claims are to be understood as being inclusive of variation in the measured quantity as would be expected by the skilled artisan making the measurement and exercising a level of care commensurate with the objective of the measurement and the precision of the associated measuring equipment. Accordingly, unless otherwise indicated to the contrary, the numerical parameters set forth in the specification and claims are approximations that may vary depending upon the desired properties sought to be obtained by the disclosure. At the very least each numerical parameter should be construed in light of the number of reported significant digits and by applying ordinary rounding techniques. For example, for recited pH, temperature, amounts, or concentrations, these values may vary by amounts that do not have any significant effect on the resulting structure, stability, activity, or result-effective variable or parameter.

Notwithstanding that the numerical ranges and parameters setting forth the broad scope of the disclosure are approximations, the numerical values set forth in the specific examples are reported as precisely as possible. One of skill will appreciate that all numerical values inherently contain a range necessarily resulting from the standard deviation found in their respective testing measurements.

As used herein, the term “antibody” or its plural, “antibodies”, also known as immunoglobulins, encompass full-length antibody sequences including, for example, monoclonal antibodies (including full-length monoclonal antibodies), polyclonal antibodies, multispecific antibodies formed from at least two different epitope binding fragments, bispecific antibodies, human antibodies, and humanized antibodies. Antibody “fragments” (or “antigen-binding fragments”, “binding fragments”, “epitope-binding fragments”, and the like) as described herein typically refer to any antibody sequence that is less than the full-length antibody sequence, and still exhibits specific binding activity to the target antigen. In example embodiments, antibody fragments typically comprise at least a combination of three CDR sequences of a heavy chain variable domain (HCDR1, HCDR2, HCDR3) and at least three CDR sequences of a light chain variable domain (LCDR1, LCDR2, LCDR3). Some non-limiting examples of antibody fragments include single-chain Fvs (scFv), single chain Fv-Fc (scFv-Fc), single-chain antibodies, single domain antibodies, domain antibodies, Fab fragments, F(ab′)2 fragments, camelised antibodies, antibody fragments that exhibit the desired biological activity (e.g. the antigen binding portion), disulfide-linked Fvs (dsFv), and anti-idiotypic (anti-Id) antibodies, intrabodies, and epitope-binding fragments of any of the above. In some embodiments, the disclosure provides antibodies that include immunoglobulin molecules and immunologically active fragments of immunoglobulin molecules, i.e., molecules that contain at least one antigen-binding site. Antibodies and fragments thereof may also include peptide fusions with antibodies or portions thereof such as a protein fused to an Fc domain.

Immunoglobulin molecules can be of any isotype (e.g., IgG, IgE, IgM, IgD, IgA and IgY), subisotype (e.g., IgG1, IgG2, IgG3, IgG4, IgAQ1 and IgA2) or allotype (e.g., Gm, e.g., G1m(f, z, a or x), G2m(n), G3m(g, b, or c), Am, Em, and Km(1, 2 or 3)). Antibodies and fragments thereof may be derived from any mammal, including, but not limited to, humans, monkeys, pigs, horses, rabbits, dogs, cats, mice, etc., or other animals such as birds (e.g. chickens).

In some aspects, the disclosure provides assay, systems, kits, and methods that comprise one or more antibodies, or compositions comprising the same. In some exemplary embodiments, the antibodies are IgGs, scFvs, Fab, monoclonal antibodies (mAbs) or single chain Fv-Fc (scFv-Fc) antibodies. A typical or conventional mAb comprises two heavy chain subunits and two light chain subunits. Each mAb heavy chain contains one variable domain (VH) which contributes to antigen binding and a constant domain (CH) made up of three or four subregions (C_{H}1, C_{H}2, C_{H}3, C_{H}4). The VH comprises three complementarity-determining regions (CDRs), HCDR1, HCDR2, and HCDR3. Each mAb light chain contains one variable domain (VL) and one constant domain (CL). The VL comprises three CDRs, LCDR1, LCDR2, and LCDR3. There are two isotypes of light chain constant domains, kappa (κ) and lambda (λ), found in mammals. Disulfide bonds join each C_{H}1 domain to one CL domain, and join C_{H}2 domains to one another. Five types of heavy chains (α, δ, ε, γ, and μ) are found in different classes of antibodies (IgA, IgD, IgE, IgG, and IgM). mAb heavy chains have hinge regions that confer structural flexibility and mobility.

The “Fc” region encompasses domains from the constant region of the heavy chain of an immunoglobulin, including a fragment, analog, variant, mutant, or derivative thereof. Suitable immunoglobulins include IgG1, IgG2, IgG3, IgG4, and other classes such as IgA, IgD, IgE and IgM. The Fc region may be a native sequence Fc region or an altered Fc region. The Fc region of an immunoglobulin generally comprises two constant domains, a C_{H}2 domain and a C_{H}3 domain. The “Fv” region encompasses the VH and VL domains of an immunoglobulin. As used herein, “scFv-Fc” antibodies refer to a fusion protein of a single VH and a single VL domain, connected with a hinge region, and the C_{H}2 domain and the C_{H}3 domain of a single CH domain.

The antibodies or antigen-binding fragments thereof generally described herein can comprise binding domains that bind to an epitope of a target analyte. “Binding domain” or “binding sequence” may be interchangeably used with an antibody fragment or “antigen-binding fragment”, and as used herein refers to a portion of an antibody sequence that is adequate and sufficient to bind to or to interact with a target structure, antigen, or epitope. In some embodiments the antibody and/or antibody composition in accordance with the disclosure can comprise a commercially available antibody that is conjugated to an enzyme (e.g., ALP).

### Exemplary Target Analytes

In some aspects, this disclosure provides assay substrates, formulations, methods, and/or systems that can be used in methods comprising measuring concentration of one or more target analytes. Exemplary analytes include troponin (such as cardiac troponin I), alpha-fetoprotein (AFP), thyroid stimulating hormone (TSH), free thyroxine (T4), creatinine kinase (CK-MB), sex hormone binding globulin (SHBG), and cancer antigen 19-9 (CA 19-9, or “GI monitor”). Numerous other analytes may also be detected by immunoassay and the analytes listed herein are provided as examples and are not intended to be limiting.

### Troponin

In an aspect, the disclosure provides a method that comprises measuring serum or plasma concentration of cardiac troponin I (cTnI) in a blood sample. cTnI is a recognized analyte that is associated with cardiac health, especially as an indicator of heart damage arising from conditions such as heart attack. cTnI can also be associated with other diseases associated with troponin release including, for example, acute pulmonary embolism, heart failure, myocarditis, and end stage renal disease. The method and assay disclosed herein may provide improved sensitivity, precision, or reproducibility or combinations thereof, as well as improvements associated with therapeutic interventions.

In some embodiments, the troponin can be detected using an anti-cTnI antibody. In further embodiments, the anti-cTnI antibody is preferably a monoclonal anti-cTnI antibody.

In some embodiments, the method may include a two-site immunoenzymatic (“sandwich”) assay. For example, monoclonal anti-cTnI antibody conjugated to alkaline phosphatase may be added to a reaction vessel along with a surfactant-containing buffer and sample. After a short incubation, paramagnetic particles coated with monoclonal anti-cTnI antibody may be added. The human cTnI binds to the anti-cTnI antibody on the solid phase, while the anti-cTnI antibody-alkaline phosphatase conjugate reacts with different antigenic sites on the cTnI molecules. After incubation in a reaction vessel, materials bound to the solid phase are held in a magnetic field while unbound materials may be washed away. Then, the chemiluminescent substrate can be added to the vessel and light generated by the reaction is measured with a luminometer. The light production is typically directly proportional to the concentration of cTnI in the sample. The amount of analyte in the sample may be determined from a stored (for example, multi-point) calibration curve.

In some embodiments, the method comprises detecting cTnI using ACCESS hsTnI (available from Beckman Coulter, Inc., Brea, California).

In some embodiments, the method comprises comparing the cTnI concentrations to a concentration of cTnI in a normal population. In such embodiments, the difference between the cTnI concentrations in the tested sample (e.g., biological sample from a subject, such as a patient sample) and the normal population can be used to assess the cardiac health status of the subject.

In some aspects, the method further includes measuring the concentration of cTnI using a substrate formulation comprising a chemiluminescent compound. In one aspect, the substrate formulation may be a substrate formulation as described in U.S. Pat. No. 10,703,971. In some preferred embodiments, the substrate formulation comprises the substrate and/or the substrate formulation as described herein. In some preferred embodiments, the enzyme formulation comprises an enzyme formulation such as, for example, an ALP formulation, as described herein. In yet other preferred embodiments, the method comprises both a substrate formulation and an enzyme formulation essentially as described herein.

As further described in the illustrative Examples, the reproducibility (that is, precision and/or sensitivity) of the cTnI assay, performed on a DxI 9000 immunoassay analyzer, was markedly improved compared to the reproducibility on an Access 2 immunoassay analyzer (that is, a clinically-approved assay platform). In accordance with the disclosure, the improved assay can be attributed to one of, or a combination of, the instrument platform (e.g., including improvements such as a dispense control system and an image capture device or camera unit that may be used to provide feedback on analyzer or instrument performance in real time), the use of the substrate formulation disclosed herein, and/or the ALP formulation, which can also be associated with improved signal-to-noise ratio or both.

For example, as illustrated by the example embodiments described herein, the measurement of cTnI exhibits a coefficient of variation of less than 4% for concentrations of cTnI in plasma greater than 10 ng/L (which may also be reported as pg/mL). Additionally or alternatively, the example embodiments described herein demonstrate that the measurement of cTnI can exhibit a standard deviation (SD) of less than 0.5 ng/L for concentrations of cTnI in plasma less than 10 ng/L.

As the example embodiments described herein also illustrate, the measurement of cTnI can exhibit a coefficient of variation of less than 4% for concentrations of cTnI in serum greater than 15 ng/L. Additionally or alternatively, the embodiments demonstrate that the measurement of cTnI exhibits a standard deviation (SD) of less than 0.5 ng/L for concentrations of cTnI in serum less than 15 ng/L.

The reproducibility conditions of measurement that are used to calculate the coefficient of variation or the standard deviation (SD) or both include between-site and between-lot variance components. In some embodiments, the reproducibility conditions of measurement further include between-day, between-run, and within-run variance components.

### Alpha-Fetoprotein (AFP)

In an aspect, the disclosure provides a method that comprises measuring concentration of alpha-fetoprotein (AFP) in a biological sample, such as a sample comprising serum or amniotic fluid. AFP is a recognized analyte that is associated with certain cancers and tumors as well as fetal development, especially as an indicator of risk of congenital disability (e.g., birth defects or genetic disorders). The method may provide improved sensitivity, precision, or reproducibility or combinations thereof.

In some embodiments, the alpha-fetoprotein can be detected using an anti-AFP antibody. In further embodiments, the anti-AFP antibody is preferably a monoclonal anti-AFP antibody.

In some embodiments, the method comprises detecting AFP using an immunoassay analyzer or instrument. In some embodiments, the method comprises detecting AFP in an apparatus comprising a dispense control system. Exemplary dispense control systems in accordance with the disclosure include, but are not limited to, those described in U.S. Pat. Pub. No. 2020/0264207. In some embodiments, the method comprises detecting AFP in an immunoassay analyzer or instrument including an image capture device or camera unit that may be used to provide feedback on the analyzer or instrument performance in real time. Exemplary immunoassay analyzers or instruments including an image capture device or camera units in accordance with the disclosure include, but are not limited to, those described in U.S. Pat. No. 11,263,433. In some preferred embodiments, the method comprises detecting APF using a DxI 9000 immunoassay analyzer (available from Beckman Coulter, Inc., Brea, California).

In some embodiments, the method may include a two-site immunoenzymatic (“sandwich”) assay. For example, a sample is added to a reaction vessel with an anti-AFP antibody conjugated to alkaline phosphatase (for example, a mouse monoclonal anti-AFP-alkaline phosphatase conjugate), and particles (for example, paramagnetic particles) coated with a second anti-AFP antibody (for example, a second mouse monoclonal anti-AFP antibody). The AFP in the sample can bind to the immobilized monoclonal anti-AFP on the particle while, at the same time, the anti-AFP-alkaline phosphatase conjugate reacts with different antigenic sites on the sample AFP. After incubation in a reaction vessel, materials bound to the solid phase are held in a magnetic field while unbound materials are washed away. Then, the chemiluminescent substrate may be added to the reaction vessel and light generated by the reaction is measured with a luminometer. Typically, the light production is directly proportional to the concentration of AFP in the sample. The amount of analyte in the sample may be determined from a stored (for example, multi-point) calibration curve.

In some aspects, the method includes detecting AFP using ACCESS AFP (available from Beckman Coulter, Inc., Brea, California).

In some aspects, the method includes comparing the AFP concentrations to a concentration of AFP in a normal population. In such embodiments, the difference between the AFP concentrations in the tested sample (e.g., biological sample from a subject, such as a patient sample) and the normal population can be used to assess the health status of the subject.

In some aspects, the method further includes measuring the concentration of AFP using a substrate formulation comprising a chemiluminescent compound. In one aspect, the substrate formulation may be a substrate formulation as described in U.S. Pat. No. 10,703,971. In some preferred embodiments, the substrate formulation comprises the substrate and/or the substrate formulation as described herein. In some preferred embodiments, the enzyme formulation comprises an enzyme formulation such as, for example, an ALP formulation, as described herein. In yet other preferred embodiments, the method comprises both a substrate formulation and an enzyme formulation essentially as described herein.

As further described in illustrative Examples, the reproducibility (that is, precision and sensitivity) of the AFP assay, performed on a DxI 9000 immunoassay analyzer, was markedly improved compared to Access 2 immunoassay analyzer (that is, a clinically-approved assay platform). In accordance with the disclosure, the improved assay can be attributed to one of, or a combination of, the instrument platform (e.g., including improvements such as a dispense control system and an image capture device or camera unit that may be used to provide feedback on analyzer or instrument performance in real time), the use of the substrate formulation disclosed herein, and/or the ALP formulation, which can also be associated with improved signal-to-noise ratio or both. Also as illustrated in the Examples below, the assay formulations (e.g., conjugate diluent) can allow for the improvements in the assay results and/or maintain the same results achieved with prior assays, while consuming less reagent per assay, thus providing an increased number of assays per unit of reagent, relative to the state of the art (e.g., doubling the number of tests per reagent pack (e.g., from 50 to 100 tests, or from 100 to 200 tests when reagent packs are sold in units of two).

For example, as illustrated by the example embodiments described herein, the measurement of AFP exhibits a coefficient of variation (CV) of less than 7% for concentrations of AFP greater than 10 ng/mL. Additionally or alternatively, the example embodiments described herein demonstrate that the measurement of AFP exhibits a standard deviation (SD) of less than 0.5 ng/ml for concentrations of AFP less than 10 ng/ml.

The reproducibility conditions of measurement that are used to calculate the coefficient of variation or the standard deviation (SD) or both include between-site and between-lot variance components. In some aspects, the reproducibility conditions of measurement further include between-day, between-run, and within-run variance components.

### Thyroid Stimulating Hormone (TSH)

In an aspect, the disclosure provides a method that comprises measuring serum or plasma concentration of thyroid stimulating hormone (TSH) in a blood sample. TSH is a recognized analyte that is associated with thyroid dysfunction or disease (e.g., hypothyroidism, hyperthyroidism, Hashimoto's disease, thyroid tumors, thyroid cancer, and conditions associated therewith), which can manifest as a variety of clinical indications (e.g., autoimmune-associated, body weight, temperature, strength, heart condition, mood, metabolism). The method may provide improved sensitivity, precision, or reproducibility or combinations thereof.

In some embodiments, the TSH can be detected using an anti-TSH antibody. In further embodiments, the anti-TSH antibody is preferably a monoclonal anti-TSH antibody. Generally, the method for detecting TSH can be performed in accordance with the methodology described in greater detail above (for AFP and cTnI).

In accordance with this illustrative Example, the reproducibility (that is, precision and/or sensitivity) of the TSH assay, performed on a DxI 9000 immunoassay analyzer, was at least maintained when compared to reproducibility achieved on an Access 2 immunoassay analyzer (that is, a clinically-approved assay platform). In some embodiments, it is expected that the reproducibility (that is, precision and/or sensitivity) of the TSH assay, performed on a DxI 9000 immunoassay analyzer, can be markedly improved when compared to the assay performed on an Access 2 immunoassay analyzer by one or more combinations of features disclosed herein. In accordance with the disclosure, the improved assay can be attributed to one of, or a combination of, the instrument platform (e.g., including improvements such as a a system that uses disposable tips remove a sample aliquot from a sample, a dispense control system, an image capture device and/or camera unit that may be used to provide feedback on immunoassay analyzer or instrument performance in real time, a dispenser that heats a wash fluid prior to it being added to the reaction vessel, or a mechanism that is adapted to degas a fluid), the use of the substrate formulation disclosed herein, and/or the ALP formulation, which can also be associated with improved signal-to-noise ratio or both. Also, as detailed below, the assay formulations (e.g., conjugate diluent) was sufficient to enable assay results that were similar to results achieved with prior assays, while consuming less reagent per assay and, again, providing for an increased number of assays per unit of reagent, relative to the state of the art (e.g., doubling the number of tests per reagent pack (e.g., from 100 to 200 tests, or from 200 to 400 tests when reagent packs (e.g., TSH reagent packs) are sold in units of two).

For example, the significant improvement in the number of assays per unit reagent is realized, that improvement is associated with the same or similar assay performance relative to the state of the art, exhibiting a coefficient of variation (CV) of 2-6% at typical TSH concentrations, a limit of blank (LoB) at 0.001-0.002 uIU/mL, a limit of detection (LoD) of 0.002-0.003 uIU/mL, and a limit of quantitation (LoQ) of 0.0008-0.001 uIU/mL.

The reproducibility conditions of measurement that are used to calculate the coefficient of variation or the standard deviation (SD) or both include instrument-to-instrument variance components. In some aspects, the reproducibility conditions of measurement can further include between-day, between-run, between-site, between-lot, and within-run variance components.

Beta Human Chorionic Gonadotropin (βhCG)

In an aspect, the disclosure provides a method that comprises measuring serum or plasma concentration for quantification of beta human chorionic gonadotropin (βhCG), also known as a βhCG test, in a blood sample. βhCG is a recognized analyte that is associated with pregnancy status, and is sometimes referred to as the pregnancy hormone because it is made by placental cells. As such, the method described herein may provide improved sensitivity, precision, or reproducibility or combinations thereof for tests that indicate pregnancy.

In some embodiments, the βhCG can be detected using an anti-βhCG antibody. In further embodiments, the anti-βhCG antibody is preferably a monoclonal anti-βhCG antibody. Generally, the method for detecting βhCG can be performed in accordance with the methodology described in greater detail above (for AFP and cTnI).

In accordance with this illustrative Example (e.g., Example 3), the reproducibility (that is, precision and/or sensitivity) of the βhCG assay, performed on a DxI 9000 immunoassay analyzer, was at least maintained when compared to the reproducibility obtained on an Access 2 immunoassay analyzer (that is, a clinically-approved assay platform). In some embodiments, it is expected that the reproducibility (that is, precision and/or sensitivity) of the βhCG assay, performed on a DxI 9000 immunoassay analyzer, can be markedly improved when compared to the assay performed on an Access 2 the reproducibility by one or more combinations of features disclosed herein. In accordance with the disclosure, the improved assay can be attributed to one of, or a combination of, the instrument platform (e.g., including improvements such as a dispense control system, degassing module, and an image capture device or camera unit that may be used to provide feedback on analyzer or instrument performance in real time), the use of the substrate formulation disclosed herein, and/or the ALP formulation, which can also be associated with improved signal-to-noise ratio or both. Also, as detailed below, the assay formulations (e.g., conjugate diluent) was sufficient to enable assay results that were similar to results achieved with prior assays, while consuming less reagent per assay and, providing for an increased number of assays per unit of reagent, relative to the state of the art (e.g., doubling the number of tests per reagent pack (e.g., from 50 to 100 tests, or from 100 to 200 tests when reagent packs are sold in units of two).

For example, the significant improvement in the number of assays per unit reagent is realized with the same or similar assay performance relative to the state of the art, exhibiting a coefficient of variation (CV) of 2.5-5% at typical hCG concentrations (that is, above LoQ), a limit of blank (LoB) at 0.0-0.1 mIU/mL, a limit of detection (LoD) of 0.2 mIU/mL, and a limit of quantitation (LoQ) of 0.1-0.2 mIU/mL.

The reproducibility conditions of measurement that are used to calculate the coefficient of variation or the standard deviation (SD) or both include instrument-to-instrument variance components. In some aspects, the reproducibility conditions of measurement can further include between-day, between-run, between-site, between-lot, and within-run variance components.

### Free Thyroxine (T4)

In an aspect, the disclosure provides a method that comprises measuring serum or plasma concentration of free thyroxine (T4) in a blood sample. T4, much like TSH, is a recognized analyte that is associated with thyroid dysfunction or disease (e.g., hypothyroidism, hyperthyroidism and conditions associated therewith), which can manifest as a variety of clinical indications (e.g., body weight, temperature, strength, heart condition, mood, metabolism). The method may provide improved sensitivity, precision, or reproducibility or combinations thereof.

In some embodiments, the free T4 can be detected using an anti-T4 antibody. In further embodiments, the anti-T4 antibody is preferably a monoclonal anti-T4 antibody. Generally, the method for detecting free T4 can be performed in accordance with typical competitive assay methodology, and wherein the signal measured is inversely proportional to the concentration of free T4 in the sample. In some alternative embodiments, the method for detecting free T4 can comprise biotin as a specific binding agent.

In accordance with the illustrative Example detailed below, the reproducibility (that is, precision and sensitivity) of the free T4 assay, performed on a DxI 9000 immunoassay analyzer, was at least maintained when compared to the reproducibility obtained on an Access 2 immunoassay analyzer (that is, a clinically-approved assay platform). In some embodiments, it is expected that the reproducibility (that is, precision and/or sensitivity) of the free T4 assay, performed on a DxI 9000 immunoassay analyzer, can be markedly improved when compared to the assay performed on an Access 2 immunoassay analyzer by one or more combinations of features disclosed herein. In accordance with the disclosure, the improved assay can be attributed to one of, or a combination of, assay process changes (e.g., additional reaction mixing steps, improvement in dispensing volume accuracy, and the like), the instrument platform (e.g., including improvements such as a dispense control system and an image capture device or camera unit that may be used to provide feedback on analyzer or instrument performance in real time), the use of the substrate formulation disclosed herein, and/or the ALP formulation, which can also be associated with improved signal-to-noise ratio or both.

For example, the improvement in precision exhibits a coefficient of variation (CV) of 2.7-5.2% at typical free T4 concentrations, and a limit of blank (LoB) at 0.18 ng/ml. The update assay also exhibits improvements for the measurement of a limit of detection (LoD) and a limit of quantitation (LoQ) for the free T4 analyte, which features have not been routinely measured on legacy assay platforms.

The reproducibility conditions of measurement that are used to calculate the coefficient of variation or the standard deviation (SD) or both include between-site and between-lot variance components. In some aspects, the reproducibility conditions of measurement further include between-day, between-run, and within-run variance components.

### Creatinine Kinase (CK or CK-MB)

In an aspect, the disclosure provides a method that comprises measuring serum or plasma concentration of creatinine kinase (CK-MB) in a blood sample. CK-MB, similar to cTnI, is a recognized analyte that is associated with damage to cardiac tissue (as well as other CK-producing organs and tissue, such as small intestine, brain, and uterus, for example). The method may provide improved sensitivity, precision, or reproducibility or combinations thereof.

In some embodiments, the CK can be detected using an anti-CK antibody. In further embodiments, the anti-CK antibody is preferably a monoclonal anti-CK antibody. Generally, the method for detecting CK can be performed in accordance with the methodology described in greater detail above (for AFP and cTnI).

In accordance with this illustrative Example, the reproducibility (that is, precision and sensitivity) of the CK-MB assay, performed on a DxI 9000 immunoassay analyzer, is expected to be markedly improved as compared to the reproducibility obtained on an Access 2 immunoassay analyzer (that is, a clinically-approved assay platform). In accordance with the disclosure, the improved assay is expected to be attributed to one of, or a combination of, assay process changes, the instrument platform (e.g., including improvements such as a dispense control system and an image capture device or camera unit that may be used to provide feedback on analyzer or instrument performance in real time), the use of the substrate formulation disclosed herein, and/or the ALP formulation, which can also be associated with improved signal-to-noise ratio or both.

### Sex Hormone Binding Globulin (SHBG)

In an aspect, the disclosure provides a method that comprises measuring serum or plasma concentration of sex hormone binding globulin (SHBG) in a blood sample. SHBG is a recognized analyte that is associated with testosterone levels. The method may provide improved sensitivity, precision, or reproducibility or combinations thereof.

In some embodiments, the SHBG can be detected using an anti-SHBG antibody. In further embodiments, the anti-SHBG antibody is preferably a monoclonal anti-SHBG antibody. Generally, the method for detecting SHBG can be performed in accordance with the methodology described in greater detail above (for AFP and cTnI).

In accordance with this illustrative Example, the reproducibility (that is, precision and sensitivity) of the SHBG assay, performed on a DxI 9000 immunoassay analyzer, is expected to be markedly improved as compared to the reproducibility obtained on an Access 2 the immunoassay analyzer (that is, a clinically-approved assay platform). In accordance with the disclosure, the improved assay is expected to be attributed to one of, or a combination of, assay process changes, the instrument platform (e.g., including improvements such as a dispense control system and an image capture device or camera unit that may be used to provide feedback on analyzer or instrument performance in real time), the use of the substrate formulation disclosed herein, and/or the ALP formulation, which can also be associated with improved signal-to-noise ratio or both.

### Cancer Antigen 19-9 (CA 19-9 or “GI Monitor”)

In an aspect, the disclosure provides a method that comprises measuring serum or plasma concentration of cancer antigen 19-9 (CA 19-9, or “GI monitor”) in a blood sample. CA 19-9 is a recognized analyte that is associated with various types of cancer, particularly cancers of the digestive system, as well as liver disease. The method may provide improved sensitivity, precision, or reproducibility or combinations thereof.

In some embodiments, the CA 19-9 can be detected using an anti-CA 19-9 antibody. In further embodiments, the anti-CA 19-9 antibody is preferably a monoclonal anti-CA 19-9 antibody. Generally, the method for detecting CA 19-9 can be performed in accordance with the methodology described in greater detail above (for free T4).

In accordance with this illustrative Example, the reproducibility (that is, precision and sensitivity) of the CA 19-9 assay, performed on a DxI 9000 immunoassay analyzer, is expected to be markedly improved as compared to the reproducibility obtained on an Access 2 immunoassay analyzer (that is, a clinically-approved assay platform). In accordance with the disclosure, the improved assay is expected to be attributed to one of, or a combination of, assay process changes, the instrument platform (e.g., including improvements such as a dispense control system and an image capture device or camera unit that may be used to provide feedback on analyzer or instrument performance in real time), the use of the substrate formulation disclosed herein, and/or the ALP formulation, which can also be associated with improved signal-to-noise ratio or both.

### Assay Reagents and Compositions

In an aspect, the disclosure provides compositions that comprise ALP conjugated to an antigen or antibody, wherein the compositions are formulated to optimize the stability of the ALP during storage of the compositions.

### Alkaline Phosphatase (ALP)

ALP is a homodimer. In the active site of each monomer, there are three distinct metal binding sites (M1, M2, and M3 sites). The catalytic activity of ALP is modulated by the metal content within these metal binding sites. Peak enzymatic activity is achieved when two of these sites (M1 and M2) are occupied by Zn²⁺ and the third site (M3) is occupied by Mg²⁺.

### Alkaline Phosphatase Substrates and Chemiluminescent Compounds

Substrates that react with ALP include, for example, a combination of nitro blue tetrazolium chloride (NBT) and 5-bromo-4-chloro-3-indolyl phosphate (BCIP), p-Nitrophenyl Phosphate (PNPP), and Lumigen PPD (as found in LUMI-PHOS 530 and LUMI-PHOS Plus (Lumigen, Inc., Southfield, MI)).

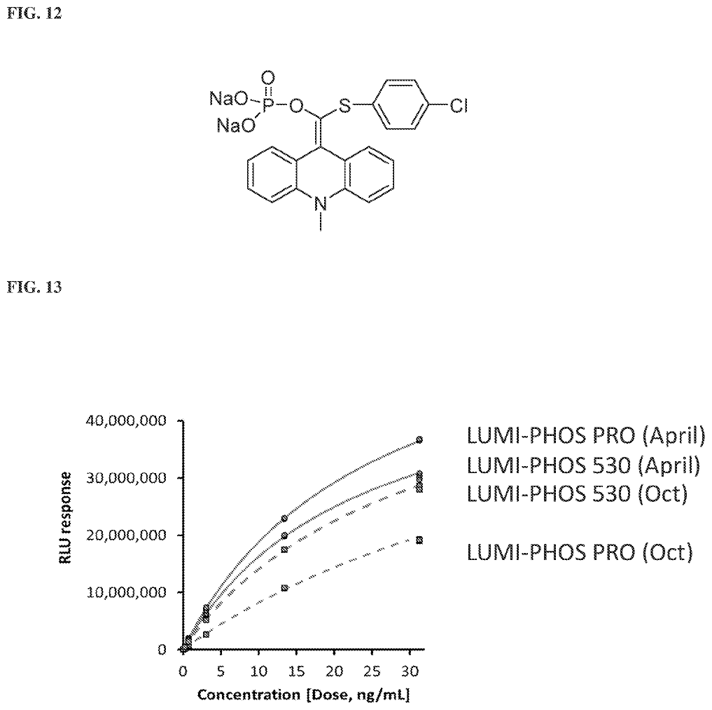

In some embodiments, the chemiluminescent substrate includes a chemiluminescent substrate and formulations thereof, as described in U.S. Pat. No. 10,703,971. In some aspects, the chemiluminescent substrate may include LUMIGEN APS-5 (Lumigen, Inc., Southfield, MI). The structure of the active component of APS-5 is shown in FIG. 12.

In some aspects, the chemiluminescent substrate includes a compound of formula I or a salt thereof:

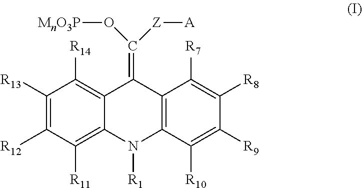

wherein A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents; R₁ is selected from the group consisting of C₅₋₁₄aryl, C₁₋₆ alkyl, C₁₋₆ haloalkyl, and C₅₋₁₄ aralkyl groups; R₇-R₁₄ are independently H, C₁₋₆ alkoxy, halo, or C₁₋₄alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring; R₁₅ is C₁₋₆ alkyl; each M is independently selected from the group consisting of H, an alkali metal, an alkaline earth metal, a transition metal, ammonium, phosphonium, an organic amine salt, and an amino acid salt; Z is O or S; and n is 0, 1, or 2. An “alkyl” group refers to a fully saturated straight or branched chain hydrocarbon containing from 1 to 12 carbon atoms, which is attached to a molecule by a single bond. Alkyl groups can include C₁-C₁₂ alkyl, C₁-C₁₀ alkyl, C₁-C₆ alkyl, C₁-C₅ alkyl all of which are inclusive of C₄ alkyls, C₃ alkyls, C₂ alkyls and C₁ alkyl (methyl). Non-limiting examples of alkyl include methyl, ethyl, n-propyl, iso-propyl, n-butyl, sec-butyl, iso-butyl, tert-butyl, n-pentyl, isopentyl, neopentyl, 1-amyl, n-hexyl, 3-methylhexyl, 2,2-dimethylpentyl, 2,3-dimethylpentyl, n-heptyl, n-octyl, n-nonyl, n-decyl, n-undecyl, and n-dodecyl. In accordance with some example embodiments an alkyl group can be optionally substituted.

“Alkoxy” refers to a group of the formula —OR, where R is an alkyl, alkenyl, or alkynyl group, as defined herein, appended to the parent molecular moiety through the oxygen atom. Non-limiting examples of alkoxy groups include methoxy, ethoxy, propoxy, 2-propoxy, butoxy, tert-butoxy, pentyloxy, and hexyloxy. In accordance with some example embodiments an alkoxy group can be optionally substituted.

The term “aryl” refers to a stable monocyclic (that is, phenyl), bicyclic, tricyclic or tetracyclic ring system containing 6 to 18 carbon atoms and at least one aromatic ring in the ring system. An aryl group can include fused and/or bridged ring systems. Non-limiting examples of aryl include aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In accordance with some example embodiments an aryl group can be optionally substituted.

The term “cycloalkyl” refers to a stable monocyclic, bicyclic, polycyclic, or spirocyclic fully saturated ring system typically comprising from 3 to 20 carbon atoms. Monocyclic ring systems are cyclic hydrocarbon groups that in some embodiments contain from 3 to 10 carbon atoms. Non-limiting examples of monocyclic cycloalkyls include cyclopropyl, cyclobutyl, cyclopentyl, cyclopentenyl, cyclohexyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (that is, a bridging group of the form —(CH₂)_{w}—, where w is 1, 2, or 3). Non-limiting examples of bicyclic and polycyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane, adamantyl, norbornyl, decalinyl. 7,7-dimethyl-bicyclo[2.2.1]heptanyl, and the like. In accordance with some example embodiments a cycloalkyl group can be optionally substituted.

The terms “heterocyclyl” and “heterocycle” refer to a 3- to 20-membered monocyclic, bicyclic, polycyclic, or spirocyclic ring system that may be saturated, unsaturated, or aromatic and that includes from 1 to 6 heteroatoms, N, O, or S. Monocyclic heterocycles include 3, 4, 5, 6, and 7 membered-rings containing at least 1 heteroatom independently selected from the group consisting of O, N, and S. The heterocycle is connected to the parent molecular moiety through any carbon atom or any nitrogen atom contained within the heterocycle. Non-limiting examples of monocyclic heterocycles include azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. Non-limiting examples of bicyclic heterocycles include 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl.

The terms “haloalkyl,” “haloalkenyl,” “haloalkynyl,” and “haloalkoxy” refer to an alkyl, alkenyl, alkynyl, or alkoxy group, as defined above, which is substituted with one or more halogen atoms at any available position. In accordance with some example embodiments any of these “halo-” groups can be optionally substituted.

In some embodiments a substrate formulation (e.g., comprising a substrate capable of generating a signal such as, for example chemiluminescence) comprises in an aqueous solution:
a) 0.01 mM-50 mM of a compound of formula I or a salt thereof:

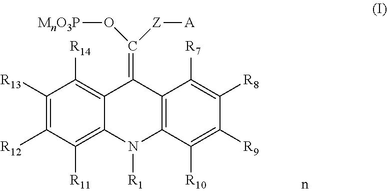

b) 0.01-200 μM of a cationic aromatic compound (CAC);
c) 1 μM-10 mM of a background reducing agent; and
d) 0.05-20 g/L of an ether-linked nonionic surfactant that does not contain a carboxylate ester group; or a hydrophilic polymer.

In some preferred embodiments, the compound or salt thereof of formula I is:

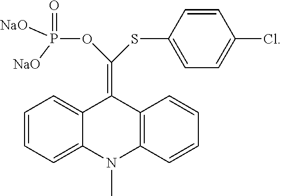

In some embodiments, a substrate formulation including the chemiluminescent substrate is used, and the substrate formulation may include a cationic aromatic compound (CAC), a background reducing agent, or a surfactant, a hydrophilic polymer, or a combination thereof. In some aspects, the surfactant may include an ether-linked nonionic surfactant that does not contain a carboxylate ester group. Additional suitable surfactants are described in WO 2021/086977.

In some embodiments, the composition comprises a cationic aromatic compound (CAC) according to formula II:

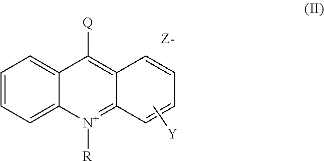

wherein:
Q is selected from the group consisting of halo, cyano, —COOR′, —COSR′, —CONR¹R², —CON(R)SO₂R″, naphthyl, anthryl, N—C₁₋₄ alkyl acridinyl, and halo substituted N—C₁₋₄ alkyl acridinyl;
R is C₁₋₄ alkyl;
R′, R″ are independently selected from the group consisting of C₁₋₆ alkyl, aryl, and alkyl substituted aryl;
R¹, R² are independently selected from the group consisting of H, C₁₋₆ alkyl, aryl, and alkylaryl; and
Z⁻ is a halide or nitrate; and
Y is selected from the group consisting of H, halo, and C₁₋₄ alkyl.

In some further embodiments the compounds according to formula II comprises a structure wherein Q is N-methyl acridinyl, halo substituted N-methyl acridinyl, naphthyl, or anthryl; R is C₁₋₄ alkyl; Z— is selected from the group consisting of Cl⁻, Br⁻, I⁻, and NO₃⁻, and Y is H or halo. Some embodiments provide exemplary CAC compounds such as those disclosed in U.S. Pat. No. 10,703,971.

In some other embodiments the substrate formulation comprises a background reducing agent that comprises one or more of lithium sulfite, sodium sulfite, potassium sulfite, lithium bisulfite, sodium bisulfite, potassium bisulfite, lithium metabisulfite, sodium metabisulfite, potassium metabisulfite, dibutylhydroxytoluene (BHT; 2,6-bis(1,10dimethylethyl)-4-methylphenol), butylated hydroxyl anisole (BHA), 3-t-butyl-4-hydroxyanisole, 3-tert-butyl-4-hydroxyanisole, and an aromatic boronic acid of formula Ar—B(OH)₂, wherein Ar is phenyl, substituted phenyl, a fused aromatic ring system that may or may or include heteroatom(s), a substituted fused aromatic ring system that may or may include heteroatom(s), wherein the substituted aryl group may have from 1-3 substituents independently selected from C₁₋₆ alkyl, halo, alkoxycarbonyl, or hydroxyl groups. In some embodiments, the background reducing agent can be selected from the group consisting of phenyl boronic acid, 4-tolyl boronic acid, 4-chloroboronic acid, 4-iodoboronic acid and 3-methoxycarbonylphenyl boronic acid, and sodium sulfite.

In further embodiments, the substrate formulation comprises an ether-linked nonionic surfactant is of formula (III):

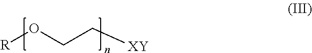

wherein R is selected from C₆₋₂₂alkyl, cycloalkyl, C₆₋₂₂alkyl-substituted cycloalkyl, and mono- or di-C₆₋₂₂alkyl-substituted phenyl; n is a number from 2-200; X is selected from O or S; and Y is selected from H or C₁₋₄ alkyl.

In some further embodiments, the substrate formulation comprises an ether-linked nonionic surfactant is selected from the group consisting of a polyoxyethylene glycol alkyl ether (BRIJ), a polyoxyethylene glycol octylphenol ether (TRITON), or a polyoxyethylene nonylphenyl ether (IGEPAL).

In further embodiments, the substrate formulation can comprise a hydrophilic polymer is according to formula (IV):

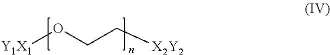

wherein X₁ and X₂ are independently selected from O, S, N or NH, or are absent; Y₁ and Y₂ are independently selected from H, H₂ or C₁₋₄ alkyl; and n is a number from 20 to 12,000. In some further embodiments, the hydrophilic polymer comprises a poly(ethylene glycol) having an average Mw within the range of 1,000 to 511,000.

In some further embodiments, the substrate formulation further comprises an anionic surfactant selected from the group consisting of C₁₀₋₂₂alkyl sulfate and C₁₀₋₂₂alkyl sulfonate. In some further embodiments, the anionic surfactant can be selected from the group consisting of sodium tetradecyl sulfate, sodium dodecyl sulfate (SDS), and sodium tridecyl sulfate (STS).

In some further embodiments, the substrate formulation further comprises an amine buffer selected from the group consisting of iris (tromethamine); AMPD (2-amino-2-methyl-1,3-propanediol); DEA (diethanolamine); AMPSO (N-(1,1-Dimethyl-2-hydroxyethyl)-3-amino-2-hydroxypropanesulfonic acid); 221-Amine (2-amino-2-methylpropan-1-ol); CHES (2-(N-cyclohexylamino) ethanesulfonic acid); glycine; and TAPS (N-Tris(hydroxymethyl)aminopropanesulfonic acid) buffer.

In some further embodiments, the substrate formulation is adapted in use to provide one or more of assay features such as, for example:
a) can achieve maximum intensity (Imax) in ≤5 minutes, ≤4 minutes, ≤2 minutes, ≤1 minute, ≤45 seconds, ≤30 seconds, ≤20 seconds, ≤10 seconds, ≤5 seconds, <4 seconds, ≤3 seconds, or ≤2 seconds after exposure to an alkaline phosphatase enzyme;
b) can exhibit ≤10% loss of original RLU after exposure to a alkaline phosphatase enzyme after storage at 4° C. for 300 days;
c) exhibits ≥90%, or ≥95% retained activity (RLU), compared to original RLU, when stored at 4° C. for 300 days or more;
d) can exhibit ≥90% retained activity when stored at 4° C. for 400 days or more; and/or
e) can exhibit a signal change in (%) per day after 15 days when stored at 30° C. of <−0.50%/day.

In any of the above-described embodiments, the substrate formulation produces chemiluminescence in the presence of a phosphatase enzyme, such as the ALP formulations described herein.

### Optimization of the ALP-Conjugate Composition

Newer ALP-reactive substrates, such as those described in U.S. Pat. No. 10,703,971 and APS-5 (FIG. 12) exhibit a faster turnaround time (TAT) compared to commercially available substrates containing compounds such as LUMI-PHOS 530. This faster substrate TAT allows for faster assay TAT, but it provides less time for ALP to achieve optimal function in a reaction solution prior to the assay being read.

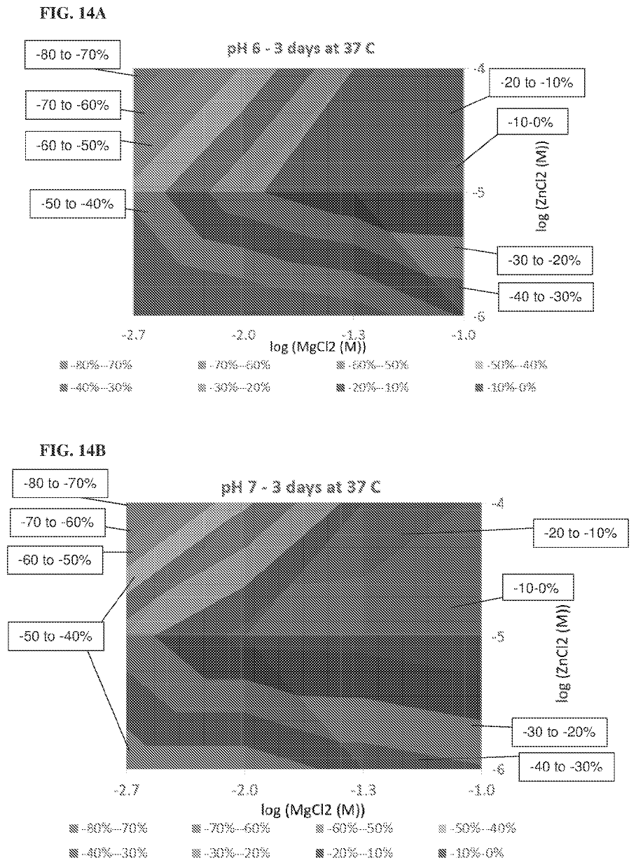

If ALP activity is not maintained prior to the addition of the ALP to the substrate, a dose bias may be observed. As shown in FIG. 13, in an assay that includes LUMI-PHOS 530 (LP530), little or no dose bias is observed. In an assay that includes LUMI-PHOS PRO, an ALP substrate described in U.S. Pat. No. 10,703,971, a dose bias was observed as a result of loss of ALP activity during storage of the enzyme.

A dose bias may be particularly detrimental when the ALP is used in the context of an immunoassay, to detect the presence or absence or concentration of an analyte. During immunoassays, the relationship between the measured signal and the concentration of the measured analyte must typically be established during a process known as assay calibration. Assay calibration includes the production of a standard curve, by using known concentrations of the analyte being assayed. If, however, the activity of the ALP in the samples used for the calibration curve is not the same as the activity of the ALP in the samples being tested, inconsistent assay readings can result.

In some embodiments the storage of ALP in solutions that contain excess concentrations of Zn²⁺ results in the displacement of Mg²⁻ from the enzyme's active site and inhibition of enzyme activity.

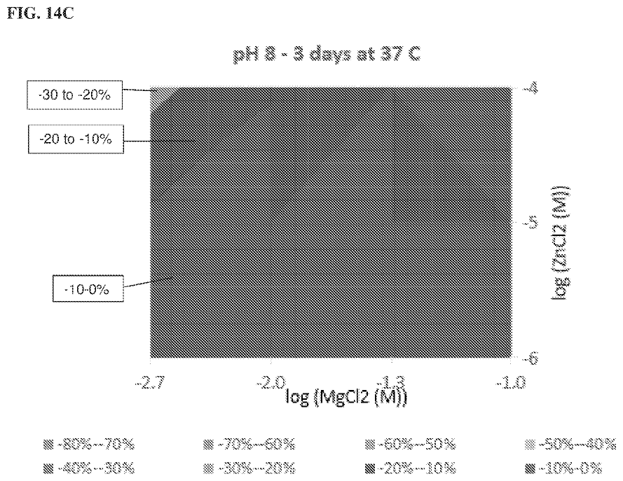

In some other embodiments that ALP inhibition may be eliminated by optimizing the long-term ALP storage solutions. Specifically, the inhibition of ALP activity may be decreased to acceptable levels or even avoided by ensuring that the storage conditions-namely Zn²⁺ and Mg²⁺ concentrations—are optimized so that free Zn²⁺ and Mg²⁺ concentrations are aligned with the apparent dissociation constants between these ions and the M3 site of ALP. And, as further described in Example 2, pH is a major driver in the interaction between these metal ions (Zn²⁺ and Mg²⁺) and the M3 site of the enzyme. Thus, as shown in Example 2 and FIG. 14, the Zn²⁺ and Mg²⁺ concentrations necessary to achieve long-term stability of ALP-conjugated antibodies or antigens during storage are pH dependent.

Because immunoassays are conducted at specific pHs and because antibodies and antigens conjugated to ALP need to be stored at specific pHs to maintain protein conformations, ALP conjugated to antibodies and antigens must be stored at a pH at which the ranges of concentrations of Zn²⁺ and Mg²⁺ that can be used to maintain enzymatic activity are very narrow.

### Compositions

In embodiments of this aspect, this disclosure describes a composition that includes an antibody conjugated to ALP or an antigen conjugated to ALP; a buffer; a zinc salt; and a magnesium salt (also referred to herein as an “antibody composition”). In some aspects, the composition may further include albumin including, for example, bovine serum albumin (BSA), human serum albumin, casein, hydrolyzed casein, or ovalbumin.

In some aspects, when the pH of the composition is in a range of 5.5 to 6.4, the concentration of free Zn²⁺ is in a range of 5 μM to 1 mM and the concentration of free Mg²⁺ is in a range of 150 times the concentration of free Zn²⁺ to 3500 times the concentration of free Zn²⁺. In some aspects, the concentration of free Zn²⁺ may be in a range of 5 μM to 100 μM, in a range of 5 μM to 500 μM, in a range of 50 μM to 150 μM, or in a range of 100 M to 500 μM. In some aspects, the concentration of free Mg²⁺ may be in a range of 700 times the concentration of free Zn²⁺ to 3500 times the concentration of free Zn²⁺, the concentration of free Mg²⁺ may be in a range of 700 times the concentration of free Zn²⁺ to 1500 times the concentration of free Zn²⁺, the concentration of free Mg²⁺ may be in a range of 500 times the concentration of free Zn²⁺ to 1500 times the concentration of free Zn²⁺, the concentration of free Mg²⁺ may be in a range of 150 times the concentration of free Zn²⁺ to 1500 times the concentration of free Zn²⁺, the concentration of free Mg²⁺ may be in a range of 150 times the concentration of free Zn²⁺ to 1000 times the concentration of free Zn²⁺, or the concentration of free Mg²⁺ may be in a range of 150 times the concentration of free Zn²⁺ to 500 times the concentration of free Zn²⁺.

In some aspects, when the pH of the composition is in a range of 6.5 to 7.4, the concentration of free Zn²⁺ is in a range of 1 μM to 5 mM and the concentration of free Mg²⁺ is in a range of 200 times the concentration of free Zn²⁺ to 3500 times the concentration of free Zn²⁺.

In some aspects, when the pH of the composition is in a range of 7.5 to 8.4, the concentration of free Zn²⁺ is in a range of 100 nM to 300 μM and the concentration of free Mg²⁺ is in a range of 100 times the concentration of free Zn²⁺ to 1000 times the concentration of free Zn²⁺.

In some aspects, free magnesium and/or free zinc may be assessed by first removing larger molecules from the storage solution by way of size exclusion chromatography, dialysis, or other means. In some aspects, size exclusion chromatography is preferred. Once this separation is complete, quantification may be accomplished via mass spectroscopy or colorimetry, for example. For instance, 5-bromo-paps may be used as a colorimetric indicator for quantification of zinc. In some aspects, mass spectroscopy is preferred.

In some aspects, the enzymatic activity of the ALP varies by less than 10% of peak activity when the composition is stored at 37° C. for up to 3 days. Incubation of ALP (coated onto PMPs or conjugated onto an antibody or antigen) for 2-3 days at 37° C. has been demonstrated to achieve similar results to incubation of ALP (similarly coated or conjugated) for 3-6 months at 2° C.-8° C.

In some aspects, the enzymatic activity of the ALP varies by less than 10% of peak activity when the composition is stored at a temperature in a range of 2° C. to 6° C. for up to 6 months, up to 9 months, or up to 1 year.

The ALP may include any suitable ALP. In some embodiments, the ALP is preferably an ALP suitable for use in an immunoassay. In some aspects, the ALP may include bovine intestinal ALP.

The buffer may include any suitable buffer. Exemplary buffers include ACES, HEPES, MES, Phosphate, TRIS, ADA, PIPES, MOPSO, BTP, BES, or Bis-TRIS, or combinations thereof. In some aspects, the buffer may include ACES, HEPES, MES, Phosphate, or TRIS. In some aspects, however, phosphate buffer may be excluded because phosphate may alter the interaction between the metal-binding sites in ALP and one or more metal ions.

The zinc salt may include any suitable zinc salt. Exemplary zinc salts include ZnSO₄ and ZnCl₂. In an exemplary aspect, the zinc salt includes ZnCl₂.

The magnesium salt may include any suitable magnesium salt. Exemplary magnesium salts include MgSO₄, MgCl₂ and Mg pidolate. In an exemplary aspect, the magnesium salt includes MgCl₂.

An antibody conjugated to ALP may include any antibody suitable for use in an immunoassay. The term “antibody” as used herein refers to a molecule that contains at least one antigen binding site that immunospecifically binds to a particular antigen target of interest. The term “antibody” thus includes but is not limited to a full length antibody and/or its variants, a fragment thereof including an antigen-binding fragment thereof, peptibodies and variants thereof, monoclonal antibodies (including full-length monoclonal antibodies), polyclonal antibodies, multispecific antibodies (for example, bispecific antibodies) formed from at least two intact antibodies, human antibodies, humanized antibodies, and antibody mimetics that mimic the structure and/or function of an antibody or a specified fragment or portion thereof, including single chain antibodies and fragments thereof. An antibody of the present disclosure thus encompasses antibody fragments including antibody fragments capable of binding to a biological molecule or portions thereof, including but not limited to Fab, Fab′ and F(ab′)₂, pFc′, Fd, a single domain antibody (sdAb), a variable fragment (Fv), a single-chain variable fragment (scFv) or a disulfide-linked Fv (sdFv); a diabody or a bivalent diabody; a linear antibody; a single-chain antibody molecule; and a multispecific antibody formed from antibody fragments. The antibody may be of any isotype (for example, IgG, IgE, IgM, IgD, IgA and IgY), class (for example, IgG1, IgG2, IgG3, IgG4, IgAQ1 and IgA2), or subclass. The antibody may be from any source including, for example, human, rodent, rabbit, cow, sheep, pig, dog, other mammals, chicken, other avians, etc.

In some exemplary embodiments, the antibody conjugated to ALP includes an anti-troponin antibody, an anti-thyroid-stimulating hormone (TSH) antibody, anti-βhCG antibody, an anti-alpha-fetoprotein (AFP) antibody, an anti-sex hormone-binding globulin (SHBG) antibody, an anti-CA 19-9 antibody (ACCESS GI Monitor Assay), or an anti-creatine kinase-MB (CKMB) antibody, as well as other target antigens described herein (e.g., T4, etc.).

In some aspects, the antibody conjugated to ALP may be an antibody that needs to be maintained at a pH of less than 8, a pH of less than 7.5, a pH of less than 7, or a pH of less than 6.5 to maintain protein conformation and/or integrity of the antibody.

An antigen conjugated to ALP may include any antigen suitable for use in an immunoassay. In some exemplary aspects, the antigen conjugated to ALP includes a thyroxine (T4) antigen or a triiodothyronine (T3) antigen.

In some aspects, the antigen conjugated to ALP may be an antigen that needs to be maintained at a pH of less than 8, a pH of less than 7.5, a pH of less than 7, or a pH of less than 6.5 to maintain protein conformation and/or integrity of the antigen.

In some aspects, including, for example, when the antibody conjugated to ALP includes an anti-troponin antibody, the pH of the composition may be in a range of 5.5 to 6.4; the concentration of free Zn²⁺ may be in a range of 100 μM to 500 μM; and the concentration of free Mg²⁺ may be in a range of 150 times the concentration of free Zn²⁺ to 500 times the concentration of free Zn²⁺. In an exemplary embodiment, further described in Example 3D, the pH of the composition is 6, the composition includes 35 mM MgCl₂, 250 μM ZnCl₂, and a ratio of free [Mg²⁺ ]/free [Zn²⁺ ] of 200.

In some aspects, including, for example, when the antibody conjugated to ALP includes an anti-TSH antibody, the pH of the composition may be in a range of 5.5 to 6.4; the concentration of free Zn²⁻ may be in a range of 5 μM to 100 μM; and the concentration of free Mg²⁺ may be in a range of 500 times the concentration of free Zn²⁺ to 1500 times the concentration of free Zn²⁺. In an exemplary embodiment, further described in Example 3E, the pH of the composition is 6, the composition includes 8 mM MgCl₂, 10 μM ZnCl₂, and a ratio of free [Mg²⁺ ]/free [Zn²⁺] of 800.

In some aspects, including, for example, when the antibody conjugated to ALP includes an anti-AFP antibody, the pH of the composition may be in a range of 5.5 to 6.4; the concentration of free Zn²⁺ may be in a range of 50 μM to 150 μM; and the concentration of free Mg²⁺ may be in a range of 150 times the concentration of free Zn²⁺ to 500 times the concentration of free Zn²⁺. In an exemplary embodiment, further described in Example 3A, the pH of the composition is 6, the composition includes 15 mM MgCl₂, 85 μM ZnCl₂, and a ratio of free [Mg²⁺]/free [Zn²⁺] of 176.

In some aspects, including, for example, when the antibody conjugated to ALP includes an anti-βhCG antibody, the pH of the composition is in a range of 5.5 to 6.4; the concentration of free Zn²⁺ may be in a range of 5 μM to 100 μM; and the concentration of free Mg²⁺ may be in a range of 700 times the concentration of free Zn²⁺ to 1500 times the concentration of free Zn²⁺. In an exemplary embodiment, further described in Example 3B, the pH of the composition is 6, the composition includes 32 mM MgCl₂, 35 μM ZnCl₂, and a ratio of free [Mg²⁺]/free [Zn²⁺] of 914.

In some aspects, including, for example, when the antigen conjugated to ALP includes a triiodothyronine (T3) antigen, the pH of the composition may be in a range of 5.5 to 6.4; the concentration of free Zn²⁺ may be in a range of 5 μM to 100 μM; and the concentration of free Mg²⁺ may be in a range of 150 times the concentration of free Zn²⁺ to 500 times the concentration of free Zn²⁺. In an exemplary embodiment, further described in Example 3C, the pH of the composition is 6, the composition includes 2 mM MgCl₂, 10 μM ZnCl₂, and a ratio of free [Mg²⁺]/free [Zn²⁺] of 200.

### Methods of Using the Compositions

In accordance with other aspects and embodiments discussed herein, this disclosure provides methods of using the compositions described herein that include an antibody conjugated to ALP or an antigen conjugated to ALP in an immunoassay.

Any suitable immunoassay method may be used. Non-limiting immunoassay methods include a two-site immunoenzymatic (“sandwich”) assay and a two-step enzyme immunoassay. Competitive assays are also envisioned, and are exemplified herein. In some embodiments, the immunoassay may preferably be performed on an immunoassay analyzer or instrument.

In some embodiments, a method of using the composition may include contacting an analyte with at least a composition including at least one reagent to form a mixture in a container, incubating the mixture; adding a substrate composition including a chemiluminescent compound to the container; and measuring light generated by the reaction after adding the substrate composition to the container. In some aspects, the method further includes a wash step prior to adding the substrate composition to the container. The light may be measured by placing the container in a luminometer. In some embodiments of the methods, one or more composition(s) and/or reagent(s) and/or samples can be combined, contacted, and/or mixed with one or more combinations of compositions, reagents and/or sample. In some embodiments of the methods, a “reaction mixture” comprises a combination of elements that provide for a reaction, under conditions that allow for such a reaction. In some further embodiments a reaction mixture can generate light. In yet further embodiments a reaction mixture can generate chemiluminescence. In such embodiments a reaction mixture can comprise a sample, a composition comprising an antibody to a target antigen that is conjugated to an enzyme (e.g., ALP) or an antigen that is conjugated to an enzyme (e.g., ALP), and a substrate composition comprising a chemiluminescent compound. In some embodiments of the methods described herein, one or more other mixtures can be formed prior to any reaction mixture such as, for example, a first or a second mixture that comprises sample and a composition comprising an antibody, or a sample and a substrate composition and an assay reagent, or a sample and a sample diluent, and the like.

The container may be any suitable container. In some aspects, the container may be a tube including, for example, a reaction vessel (RV). In some aspects, the tube include polyethylene.

The at least one reagent may comprise one or more reagents. In some aspects, the at least one reagent includes buffer, particles (including, for example, paramagnetic microparticles), capture antibody, or antibody-enzyme conjugates (for example, antibody-ALP conjugates) or antigen-enzyme conjugates (for example, antigen-ALP conjugates), or any combination thereof.

In some aspects, the reagent comprises particles. In some aspects, the reagent comprises at least 0.1 μg particles, at least 0.2 μg particles, at least 0.3 μg particles, at least 0.4 μg particles, at least 0.5 μg particles, at least 0.6 μg particles, at least 0.7 μg particles, at least 0.8 μg particles, at least 0.9 μg particles, at least 1.0 μg particles, at least 2.0 μg particles, at least 3.0 μg particles, at least 3.0 μg particles, at least 4.0 μg particles, at least 5.0 μg particles, at least 6.0 μg particles, at least 7.0 μg particles, at least 8.0 μg particles, at least 9.0 μg particles, at least 10 μg particles, at least 15 μg particles, at least 20 μg particles, at least 25 μg particles, at least 30 μg particles, at least 35 μg particles, at least 40 μg particles, at least 45 μg particles, at least 50 μg particles, at least 60 μg particles, or at least 70 μg particles, and/or up to 10 μg particles, up to 11 μg particles, up to 12 μg particles, up to 13 μg particles, up to 14 μg particles, up to 15 μg particles, up to 16 μg particles, up to 17 μg particles, up to 18 μg particles, up to 19 μg particles, up to 20 μg particles, up to 25 μg particles, up to 30 μg particles, up to 35 μg particles, up to 40 μg particles, up to 45 μg particles, up to 50 μg particles, up to 55 μg particles, up to 60 μg particles, up to 65 μg particles, up to 70 μg particles, up to 75 μg particles, up to 80 μg particles, up to 85 μg particles, up to 90 μg particles, up to 100 μg particles, up to 110 μg particles, up to 120 μg particles, up to 130 μg particles, up to 140 μg particles, or up to 150 μg particles. For example, exemplary ranges of particles include at least 0.1 μg and up to 100 μg, at least 0.1 μg and up to 150 μg, at least 5 μg and up to 100 μg, at least 10 μg and up to 100 μg, etc. In some cases, the reagent includes particles from about 0.1 μg to about 150 μg, from about 0.2 μg to about 140 μg, from about 0.3 μg to about 130 μg, from about 0.4 μg to about 120 μg, from about 0.5 μg to about 110 μg, from about 1.0 μg to about 100 μg, from about 1.5 μg to about 90 μg, from about 2.0 μg to about 80 μg, from about 2.5 μg to about 70 μg, from about 3 μg to about 60 μg, from about 4 μg to about 50 μg, from about 0.1 μg to about 60 μg, from about 0.1 μg to about 50 μg, or from about 0.5 μg to about 50 μg. In some embodiments, the particles are microparticles, coated particles, coated microparticles, paramagnetic particles, or paramagnetic microparticles. In some embodiments, the particles are coated with antibodies, monoclonal antibodies, or target-specific antibodies. When the particles include paramagnetic particles, the particles may be separated from other components of a composition by being subjected to a magnetic field.

In some aspects, the method comprises using a reagent comprising particles to detect AFP or betaHCG. In some aspects, the reagent comprises at least 0.1 μg particles, at least 0.2 μg particles, at least 0.3 μg particles, at least 0.4 μg particles, at least 0.5 μg particles, at least 0.6 μg particles, at least 0.7 μg particles, at least 0.8 μg particles, at least 0.9 μg particles, at least 1.0 μg particles, at least 2.0 μg particles, at least 3.0 μg particles, at least 3.0 μg particles, at least 4.0 μg particles, at least 5.0 μg particles, at least 6.0 μg particles, at least 7.0 μg particles, at least 8.0 μg particles, at least 9.0 μg particles, at least 10 μg particles, at least 15 μg particles, at least 20 μg particles, at least 25 μg particles, at least 30 μg particles, at least 35 μg particles, at least 40 μg particles, at least 45 μg particles, or at least 50 μg particles, and/or up to 10 μg particles, up to 11 μg particles, up to 12 μg particles, up to 13 μg particles, up to 14 μg particles, up to 15 μg particles, up to 16 μg particles, up to 17 μg particles, up to 18 μg particles, up to 19 μg particles, up to 20 μg particles, up to 25 μg particles, up to 30 μg particles, up to 35 μg particles, up to 40 μg particles, up to 45 μg particles, up to 50 μg particles, up to 55 μg particles, up to 60 μg particles, up to 65 μg particles, up to 70 μg particles, up to 75 μg particles, up to 80 μg particles, up to 85 μg particles, up to 90 μg particles, up to 100 μg particles, up to 110 μg particles, up to 120 μg particles, up to 130 μg particles, up to 140 μg particles, or up to 150 μg particles. For example, exemplary ranges of particles include at least 0.1 μg and up to 100 μg, at least 0.1 μg and up to 150 μg, at least 5 μg and up to 100 μg, at least 10 μg and up to 100 μg, etc. In some cases, the reagent includes particles from about 0.1 μg to about 150 μg, from about 0.2 μg to about 140 μg, from about 0.3 μg to about 130 μg, from about 0.4 μg to about 120 μg, from about 0.5 μg to about 110 μg, from about 1.0 μg to about 100 μg, from about 1.5 μg to about 90 μg, from about 2.0 μg to about 80 μg, from about 2.5 μg to about 70 μg, from about 3 μg to about 60 μg, from about 4 μg to about 50 μg, from about 0.1 μg to about 60 μg, from about 0.1 μg to about 50 μg, or from about 0.5 μg to about 50 μg.

In some aspects, the method comprises using a reagent comprising particles to detect TSH. In some aspects, the reagent comprises at least 0.1 μg particles, at least 0.2 μg particles, at least 0.3 μg particles, at least 0.4 μg particles, at least 0.5 μg particles, at least 0.6 μg particles, at least 0.7 μg particles, at least 0.8 μg particles, at least 0.9 μg particles, at least 1.0 μg particles, at least 2.0 μg particles, at least 3.0 μg particles, at least 3.0 μg particles, at least 4.0 μg particles, at least 5.0 μg particles, at least 6.0 μg particles, at least 7.0 μg particles, at least 8.0 μg particles, at least 9.0 μg particles, at least 10 μg particles, at least 15 μg particles, at least 20 μg particles, at least 25 μg particles, at least 30 μg particles, at least 35 μg particles, at least 40 μg particles, at least 45 μg particles, or at least 50 μg particles, and/or up to 10 μg particles, up to 11 μg particles, up to 12 μg particles, up to 13 μg particles, up to 14 μg particles, up to 15 μg particles, up to 16 μg particles, up to 17 μg particles, up to 18 μg particles, up to 19 μg particles, up to 20 μg particles, up to 25 μg particles, up to 30 μg particles. For example, exemplary ranges of particles include at least 0.1 μg and up to 20 μg, at least 0.1 μg and up to 20 μg, at least 5 μg and up to 20 μg, at least 10 μg and up to 20 μg, etc.

In some embodiments, the microparticles comprises a paramagnetic or superparamagnetic material such as, for example, ferromagnetic iron oxide Fe₃O₄ or Fe₂O₃. The terms “paramagnetic” and “superparamagnetic” refer to materials that experience a force in a magnetic field gradient, but do not become permanently magnetized. In a specific embodiment, the support comprises iron in the form of maghemite, or Fe₂O₃. In various embodiments, the mean diameter of the microparticle is in the range of 100 nm to 22,900 nm. In a specific embodiment, the mean diameter of the microparticle is in the range of about 750 nm to about 3,000 nm. In another specific embodiment, the mean diameter of the microparticle is in the range of about 950 nm to about 1,150 nm.

In some aspects, the substrate composition may include a buffer. In some aspects, the volume of buffer in the substrate composition is at least 50 μL, at least 100 μL, at least 150 μL, at least 200 μL. In some aspects, the volume of buffer in the substrate composition is up to 100 μL, up to 150 μL, up to 200 μL, up to 250 μL, or up to 300 μL. Thus, in some exemplary embodiments, when the chemiluminescent compound is added to the reagents (including, particles, capture antibody, antibody-enzyme conjugates, and/or antigen-enzyme conjugates), the volume of buffer in the container may be up to 100 μL, or up to 150 μL, or up to 200 μL. In another exemplary embodiment, when the chemiluminescent compound is added to the reagents, the volume of buffer in the container may be in a range of 150 μL to 300 μL

In some embodiments, when the chemiluminescent compound is added to the reagents, the buffer may further include at least 10,000 molecules of analyte, at least 15,000 molecules of analyte, at least 20,000 molecules of analyte, or up to 20,000 molecules of analyte, up to 25,000 molecules of analyte, up to 35,000 molecules of analyte, or up to 50,000 molecules of analyte. For example, the buffer may include 10,000 to 20,000 molecules of analyte or 10,000 to 50,000 molecules of analyte.

In some embodiments, when the chemiluminescent compound is added to the reagents, a mass of the chemiluminescent compound is at least 2 times a mass of the reagent, at least 5 times a mass of the reagent, at least 10 times a mass of the reagent, at least 20 times a mass of the reagent, at least 30 times a mass of the reagent, at least 40 times a mass of the reagent, at least 50 times a mass of the reagent, at least 60 times a mass of the reagent, at least 70 times a mass of the reagent, at least 80 times a mass of the reagent, or at least 90 times a mass of the reagent. In some embodiments, when the chemiluminescent compound is added to the reagents, a mass of the chemiluminescent compound is up to 50 times a mass of the reagent, up to 70 times a mass of the reagent, up to 80 times a mass of the reagent, up to 90 times a mass of the reagent, up to 100 times a mass of the reagent, or up to 150 times a mass of the reagent.

In some embodiments, when the chemiluminescent compound is added to the reagents, a ratio of the chemiluminescent compound to the enzyme is at least 1 μg of chemiluminescent compound to 1500 molecules of enzyme.

In some embodiments, when the chemiluminescent compound is added to the reagents (for example, to the particles, microparticles, paramagnetic particles, paramagnetic microparticles, etc.), the chemiluminescent compound is present at a concentration of at least at least 0.01 g/L, at least 0.02 g/L, at least 0.03 g/L, at least 0.04 g/L, at least 0.05 g/L, at least 0.1 g/L, at least 0.15 g/L, at least 0.2 g/L, at least 0.3 g/L, at least 0.4 g/L, at least 0.5 g/L, at least 1.0 g/L, or at least 2.0 g/L, and/or up to 0.15 g/L, up to 0.2 g/L, up to 0.5 g/L, up to 1 g/L, up to 2 g/L, up to 3 g/L, up to 4 g/L, up to 5 g/L, up to 7 g/L, up to 9 g/L, up to 10 g/L, up to 12 g/L, up to 15 g/L, up to 20 g/L, up to 25 g/L, up to 35 g/L up to 50 g/L. For example, when the chemiluminescent compound is added to the reagents, the chemiluminescent compound may be present at a concentration in a range of 0.01 g/L to 50 g/L or in a range of 0.1 g/L to 10 g/L. In some cases, the chemiluminescent compound is present at a concentration in a range of from about 0.005 to about 20 g/L, from about 0.01 to about 15 g/L, from about 0.01 to about 10 g/L, from about 0.01 to about 9 g/L, from about 0.01 to about 8 g/L, from about 0.01 to about 7 g/L, from about 0.01 to about 6 g/L, from about 0.01 to about 5 g/L, from about 0.05 to about 10 g/L, from about 0.05 to about 8 g/L, from about 0.05 to about 6 g/L, from about 0.05 to about 4 g/L, from about 0.1 to about 10 g/L, from about 0.1 to about 7 g/L, from about 0.1 to about 8 g/L, from about 0.1 to about 7 g/L, from about 0.1 to about 6 g/L, or from about 0.5 to about 10 g/L. In some cases, the chemiluminescent compound is present at a concentration of 0.1 g/L to 0.2 g/L, 0.1 to 0.5 g/L, 0.1 to 1 g/L, 0.1 to 2 g/L, 0.1 to 3 g/L, 0.1 to 4 g/L, 0.1 to 5 g/L, 0.1 to 6 g/L, 0.1 to 7 g/L, 0.1 to 8 g/L, 0.1 to 9 g/L, 0.1 to 10 g/L, 0.1 to 11 g/L, 0.1 to 12 g/L, or 0.1 to 15 g/L. In some cases, the chemiluminescent compound is present at a concentration of 0.05 to 0.5 g/L, 0.1 to 0.5 g/L, 0.2 to 0.5 g/L, 0.3 to 0.5 g/L, 0.4 to 0.5 g/L, 0.1 to 0.4 g/L, 0.1 to 0.3 g/L, or 0.1 to 0.2 g/L. The chemiluminescent compound can be any compound known in the art. In some cases, the chemiluminescent compound is a chemiluminescent substrate, or salt thereof, of formula I:

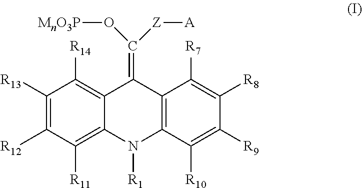

A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
R₁ is selected from the group consisting of C₅₋₁₄aryl, C₁-6 alkyl, C₁₋₆ haloalkyl, and C₅₋₁₄ aralkyl groups;
R₇-R₁₄ are independently H, C₁₋₆ alkoxy, halo, C₁₋₄alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
R₁₅ is C₁₋₆ alkyl;
each Mis independently selected from the group consisting of H, an alkali metal, alkaline earth metal, transition metal, ammonium, phosphonium, organic amine salt, and an amino acid salt;
Z is O or S; and
n is 0, 1, or 2.

In some aspects, the mass of the chemiluminescent compound used in the methods provided herein is at least 0.1 times, at least 0.5 times, at least 0.8 times, at least the same as, at least 1.5 times, at least two times, at least three times, at least five times, at least ten times, at least twenty-five times, at least fifty times, at least one hundred times, or at least one hundred and fifty times a mass of the at least one reagent. In some embodiments, the at least one reagent is a particle, microparticle, paramagnetic particle, paramagnetic microparticle, coated particle, coated paramagnetic particle, coated microparticle, or coated paramagnetic microparticle.

In some aspects, the at least one reagent includes at least 0.01 μg and up to 1.0 μg, at least 0.01 μg and up to 0.7 μg, or at least 0.01 μg and up to 0.5 μg capture antibody. The at least one reagent can include at least 0.001 μg, at least 0.005 μg, at least 0.01 μg, at least 0.02 μg, at least 0.03 μg, at least 0.04 μg, at least 0.05 μg, at least 0.06 μg, at least 0.07 μg, at least 0.08 μg, at least 0.09 μg, at least 0.1 μg, at least 0.2 μg, or at least 0.3 μg, and/or up to 0.05 μg, up to 0.06 μg, up to 0.07 μg, up to 0.08 μg, up to 0.09 μg, up to 0.1 μg, up to 0.2 μg, up to 0.3 μg, up to 0.4 μg, up to 0.5 g, up to 0.6 μg, up to 0.7 μg, up to 0.8 μg, up to 0.9 μg, or up to 1.0 μg captured antibody. Additional amounts of capture antibody may also be envisioned including, for example, at least 0.01 μg and up to 1.0 μg, at least 0.01 μg and up to 0.5 μg, 0.01 μg and up to 0.3 μg, etc.

In some embodiments, the light is measured up to 5 minutes, up to 4 minutes, up to 3 minutes, up to 2 minutes, up to 1 minute, up to 56 seconds, up to 55 seconds, up to 50 seconds, or up to 48 seconds after adding the substrate composition to the container. In contrast to older chemiluminescent substrates which required waiting longer (for example, greater than 5 minutes or greater than 6 minutes) after adding the chemiluminescent substrate to the container before measuring the light emitted, the use of newer ALP-reactive substrates that exhibit a faster turnaround time (TAT) allow for the light to be measured more quickly. As further discussed herein, the compositions of this disclosure decrease the inhibition of ALP activity, allowing for more accurate measurements when chemiluminescent substrates with faster TATs are used. In some embodiments, including for example, when the substrate includes APS-5, the light may, preferably be measured less than 1 minute after adding the chemiluminescent substrate to the container

In yet some further embodiments, the substrate and/or the substrate formulation provides for increased number of assay runs per reagent unit. The improvement in signal to noise and/or the brightness (or amount/intensity) of the light produced by the substrate is markedly brighter than other substrates that form the state of the art. As such, the background associated with the methods and substrate/substrate composition described herein (that is, the substrate signal with no analyte present) is typically much lower than known substrates/substrate compositions. Thus, the increased signal relative to background affords the opportunity to decrease the amount of reagents used in the reaction while retaining assay performance.

In some embodiments, the sample may be present in the immunoassay analyze or instrument as an aliquot, for example, as an aliquot in a vessel or tube. In some embodiments, the sample aliquot has a volume of up to 2 μl, up to 5 μl, up to 8 μl, up to 10 μl, up to 15 μl, up to 20 μl, up to 30 μl, up to 40 μl, or up to 50 μl. In some cases, the sample aliquot has a volume in the range of 1 μl-2 μl, 1 μl-5 μl, 1 μl-8 μl, 2 μl-10 μl, or 2 μl-20 μl, or 2 μl-50 μl. In some embodiments, the sample aliquot has a volume of at least 1 μl, at least 2 μl, or at least 3 μl.

In some embodiments, the method further comprising determining the amount of analyte in the sample. The amount of analyte may be determined, for example, from a stored, multi-point calibration curve.

In an exemplary embodiment, the method may include contacting a sample that includes the analyte with a composition described herein, wherein the composition comprises an antibody bound to ALP, to form an ALP-antibody-analyte complex. The method may further include contacting the ALP-antibody-analyte complex with an anti-analyte antibody bound to a solid phase and washing away sample unbound to the solid phase. Exemplary solid phases include polymer beads and magnetic beads including, for example, paramagnetic particles.

In another exemplary embodiment, the method may include contacting a sample including the analyte with an antibody specific for the analyte to form a complex including the analyte, washing the complex, and contacting the complex with a composition described herein, wherein the composition comprises an antigen bound to ALP.

In some embodiments, the method includes a two-site immunoenzymatic (“sandwich”) assay. For example, a sample may be added to a reaction vessel with a relevant antibody conjugated to alkaline phosphatase, and paramagnetic particles coated with a second antibody. The analyte in the sample binds to the immobilized antibody on the paramagnetic particles while, at the same time, the antibody conjugated to alkaline phosphatase reacts with different antigenic sites on the analyte. After incubation in a reaction vessel, materials bound to the solid phase are held in a magnetic field while unbound materials are washed away. Then, a chemiluminescent substrate is added to the vessel and light generated by the reaction is measured with a luminometer. The light production is directly proportional to the concentration of analyte in the sample. The amount of analyte in the sample is determined from a stored, multi-point calibration curve.

In some embodiments, the method includes a two-step enzyme immunoassay. For example, an antibody specific for the analyte coupled to biotin, sample, buffered protein solution, and streptavidin-coated solid phase may be added to a reaction vessel. During this first incubation the anti-analyte antibody coupled to biotin binds to the solid phase and the free analyte in the sample. After incubation in a reaction vessel, materials bound to the solid phase are held in a magnetic field while unbound materials are washed away. Next, buffered protein solution and an antigen-ALP conjugate are added to the reaction vessel. The antigen-ALP conjugate binds to the vacant anti-analyte antibody binding sites. After incubation in a reaction vessel, materials bound to the solid phase are held in a magnetic field while unbound materials are washed away. Then, the chemiluminescent substrate is added to the vessel and light generated by the reaction is measured with a luminometer. The light production is inversely proportional to the concentration of free analyte in the sample. The amount of analyte in the sample is determined from a stored, multi-point calibration curve.

The methods and assays provided by the disclosure are widely applicable to a number of patient populations. For example, the methods and assays can be used to assess patient populations at risk for one or more conditions, disorders, and/or diseases that are associated with a target antigen, such as the exemplary target antigens detailed herein. The methods and assays can be used to assess patient populations who suffer from one or more conditions, disorders, and/or diseases that are associated with a target antigen. The methods and assays can be used to assess patient populations who are undergoing a preventative and/or therapeutic intervention for the prevention and/or treatment of one or more conditions, disorders, and/or diseases that are associated with a target antigen, in order to assess the efficacy preventative or therapeutic intervention. Non-limiting examples of patient populations that can benefit from the methods and assays provided by the disclosure include patients who have or who are at risk of developing cancer or a tumorigenic disease; patients who are pregnant or who believe they may be pregnant; patients who are pregnant to assess fetal risk of congenital or genetic disorders; patients who have an autoimmune disorder, or one or more clinical symptoms associated with an autoimmune disorder; patients who have or who are at risk of one or more cardiac diseases, such as myocardial infarct, heart failure, myocarditis; patients who have or who are at risk of developing renal disease; patients who have or who are at risk of developing an endocrine and/or hormone imbalance such as a thyroid imbalance; patients who have or who are at risk of developing damage to tissues and organs such as heart, brain, intestine, and the uterus; andn patients who have or who are at risk of developing a sex hormone imbalance.

### Kits and Assay Compositions of Matter

In yet another aspect, the disclosure relates to a kit for detecting a target analyte or antigen in a sample. In embodiments of this aspect, a kit can comprise any of the substrates, enzymes, formulations or compositions comprising the substrates, enzymes, and formulations as generally described herein, and instructions or a label directing appropriate use. Optionally, a kit may also include one or more containers, reagents, reactants, and/or assay diluents, or other devices to facilitate use. The disclosure contemplates that all or any subset of the components for conducting research assays and/or diagnostic assays may be included in the kit. Similarly, the kit may include instructions for making one or more assay solutions comprising one or more of the assay biological samples, substrates, enzymes, formulations, or compositions thereof, under suitable conditions. In some additional example embodiments, a kit may comprise a solution, or a dried or lyophilized preparation of one or more kit components, and instructions for preparing the solution, or dried or lyophilized preparation for use (e.g., for reconstituting a lyophilized or dried product, dispensing and/or diluting a solution, etc.).

The disclosure also encompasses a finished packaged and labeled product (e.g., as a kit, or as one or more parts of a kit). Such an article of manufacture includes the appropriate unit form (e.g., concentrated assay formulations, ready to use assay formulations, etc.) in an appropriate vessel or container such as a glass vial or other container that is typically sterile and sealed. In some example embodiments containers can include, but are not limited to, vials, bottles, and/or pre-filled syringes, and the like. Optionally associated with such kit(s) and/or container(s) can be a notice in the form prescribed by a governmental agency regulating the manufacture, use or sale of the kit and/or assay components, which notice can reflect approval by an agency of the manufacture, use or sale for human use/diagnosis

As discussed above, the kit can include instructions for use or other informational material that advise the physician, clinician, and/or technician on how to appropriately interpret the results of the particular assay in use. In some further embodiments, kits exclusively for research use are contemplated. Such kits may, for example, resemble kits the kits described hereinabove, but may further include a label specifying that the kit and its use is restricted to research purposes only.

the kit comprising one or more of: a chemiluminescent substrate; an enzyme; and/or an assay reagent, or one or more compositions comprising the same, and in accordance with the aspects and embodiments of the disclosure. In some embodiments, the kits can further comprise additional assay reagents such as assay buffers, assay diluents, pH adjusting agents, or additional reporter molecules (e.g., for the indirect or detection of the target analyte or antigen). The kits can also comprise instructions for use in the practice of any one of the assays or methods disclosed herein.

### Immunoassay Analyzer or Instrument

The methods, compositions, and kits disclosed herein may be used in combination with integrated system platforms. For example, the methods, compositions, and kits may be used in combination systems commercially marketed, e.g., by Beckman Coulter such as, for example, the immunoassay analyzers including, Access 2+, UniCel DxI 600, UniCel, DxI 800, or DxI 9000 immunoassay analyzers. In some exemplary embodiments, the integrated system platform is the DxI 9000 immunoassay analyzer. The methods, compositions, and kits disclosed herein can also be used with one or more sample preparation system platforms. Similarly, the methods, compositions, and kits disclosed herein may be used with point-of-care system platforms as well as other available assay platforms. As such, the methods, compositions, and kits of the disclosure can be readily adapted for use with any number of devices, assay platforms, and instrumentation such as, for example, hand held fluorescence detectors, microfluidic devices, enzymatic detection systems, immunochromatographic strips, and lateral flow devices.

Thus, in some embodiments, a method of using a composition described herein includes using an immunoassay analyzer or instrument as described herein, or as may otherwise be known in the art. In some embodiments, the method may preferably include using a DxI 9000 immunoassay analyzer (available from Beckman Coulter, Inc., Brea, California) which comprises a number of advantageous features (e g., hardware and/or software) that can provide additional improved assay results when used in combination with the enzyme compositions and substrate compositions described herein, and in some embodiments such combinations provide for synergistic improvements in assay results.

The immunoassay analyzer or instrument may be used in combination with an enzyme and/or an enzyme composition as described herein. An immunoassay instrument or one or more features thereof may be used in combination with a substrate and/or substrate composition as described herein. Additionally or alternatively, an immunoassay analyzer or instrument, or one or more features thereof, may be used in combination with an enzyme and/or enzyme composition as described herein.

In an exemplary embodiment, the immunoassay analyzer or instrument may load sample containers into the instrument while loaded onto a sample rack such as that described in WO2018232364. As part of loading the sample containers into the immunoassay analyzer or instrument, the device may ready a barcode or other identification mark on the sample. Once the sample containers are inside the immunoassay analyzer or instrument, a portion of the sample may be aliquoted from the sample container, using a disposable tip. Exemplary disposable tips are described in U.S. Pat. Nos. D841182, D887574, and D899623. The sample may be placed in a second vessel, for example, a reaction vessel or RV. The RV including the sample aliquot may be used immediately or stored at 4° C. To build an immunoassay, the immunoassay analyzer or instrument may use a pipettor to obtain and dispense the appropriate amounts of reagents in a container that also contains all or a portion of the aliquoted sample. The reagents may be obtained from a reagent pack. Exemplary reagents include buffer, particles (including, for example, paramagnetic particles), capture antibody, antibody enzyme conjugates or antigen-enzyme conjugates, and combinations thereof. In some embodiments, the enzyme is preferably ALP. The RV including the reagents and the sample may be incubated, for example, at 37° C. to allow the components of the immunoassay to bind to each other (for example, to allow binding of an antibody or antigen to an analyte in the sample). To separate bound antibody or antigen from unbound antibody or antigen, the immunoassay analyzer or instrument will perform one or more wash steps. Such wash steps may be performed by a wash station. An exemplary wash station is described in U.S. Pat. Pub. No. 2021/0025910 A1. In some aspects, it may be preferred that the wash fluid may be heated prior to being added to the reaction vessel. The particles bound to the bound antibody or bound antigen may be separated from the wash fluid by any suitable means. When the particles are magnetic particles, the particles may preferably be separated (for example, by being subjected to a magnetic field) and washed by aggregating the particles by the magnetizing the particles, aspirating a supernatant, dispensing a wash (for example, a buffer) solution, and repeating 1-3 times. When the particles have been washed and unbound antibody or antigen removed, substrate may be added to the container to generate a signal. The container (for example, the reaction vessel) including the particles and the substrate may be incubated on the immunoassay analyzer or instrument prior to measuring the light emitted. The incubation may occur at any suitable temperature, including, for example at 37° C. The immunoassay analyzer or instrument may measure the light emitted using any suitable means. In some embodiments, a luminometer may be preferred. Exemplary luminometers that may be used in the immunoassay analyzer or instrument include those described in WO2019060375, WO2020219869, and/or U.S. Pat. No. 11,604,146. The immunoassay analyzer or instrument may further use a calibration curve to correlate the light measured to an analyte amount.

For example, in some embodiments, the immunoassay analyzer or instrument includes a system that uses disposable tips remove a sample aliquot from a sample for use on the immunoassay analyzer or instrument. Exemplary disposable tips are described in U.S. Pat. Nos. D841182, D887574, and D899623. The use of a disposable tip to remove a sample aliquot from a sample in a sample container can minimize potential contamination of the sample remaining in the sample container. Moreover, once the sample aliquot is removed from the sample in the sample container, the sample container can then be offloaded from the immunoassay analyzer or instrument and used for other purposes including for other types of clinical analysis.

In some embodiments, the immunoassay analyzer or instrument includes a dispense control system. Non-limiting dispense control systems include those described in U.S. Pat. Pub. No. 2020/0264207. The dispense control system may be used for methods for preparing a fluidic substance for evaluation. One exemplary method includes aspirating a first volume of a fluidic substance from a first vessel to a dispense tip and dispensing a second volume of the fluidic substance from the dispense tip to second vessel. The dispensing a second volume of the fluidic substance from the dispense tip to second vessel may include lowering the dispense tip into the second vessel at a first height, the first height configured such that a distal end of the dispense tip remains above a surface level in the second vessel after dispensation; at least partially dispensing the fluidic substance from the dispense tip to the second vessel; and lowering the dispense tip to a second height, the second height configured such that the distal end of the dispense tip touches the surface level in the second vessel after the dispensation. The method may further include dispensing the rest of the fluidic substance from the dispense tip to the second vessel at the second height.

In some embodiments, the immunoassay analyzer or instrument includes an image capture device or camera unit that may be used to provide feedback on analyzer or instrument performance in real time. Non-limiting immunoassay analyzers or instruments including an image capture device or camera units are described in U.S. Pat. No. 11,263,433. For example, the immunoassay analyzer or instrument may include a container carriage device configured to support one or more containers; a sample pipetting device configured to dispense a fluidic substance in at least one of the containers on the container carriage device; an image capture device configured to capture an image of at least one of the containers on the container carriage device; and at least one processing device; wherein the system is configured to: dispense, using the sample pipetting device, at least one fluidic substance into a container; capture, using the image capture device, an image of the container on the container carriage device; analyze, using the at least one processing device, the image of the container to determine a volume of the dispensed at least one fluidic substance in the container; and analyze, using the at least one processing device, the image of the container to determine a particle concentration of particles of interest based on the determined volume of fluidic substances in the container. The immunoassay analyzer or instrument may be capable of evaluating a fluidic substance in a container using a method that includes: dispensing, using a sample pipetting device, at least one fluidic substance into a container; capturing, using an image capture device, an image of at least a part of the container arranged on a container carriage device, which container carriage device is configured to support one or more containers; analyzing, using at least one computing device, the image of the container to determine a volume of the at least one dispensed fluidic substance in the container; and analyzing, using the at least one computing device, the image of the container to determine a particle concentration of particles of interest based on the determined volume of the at least one dispensed fluidic substance in the container.

In some embodiments, the immunoassay instrument or analyzer or one or more features thereof (e.g., hardware and/or software features) is generally described in International Patent Appln. PCT/US2021/065852 (WO 2022/147370).

In some particular embodiments, the method comprises a mechanism that is adapted to de-gas a fluid such as a sample comprising a fluid, a fluid assay reagent, and/or a mixture comprising a sample and one or more assay reagents or diluents. For example, the method and/or mechanism for degassing a fluid can comprise a dispenser that is adapted to include a heater and one or more tubes constructed of a material that is in fluid connection with the system. The tube or tubes may include a first end operable to be connected to a source of one or more fluids/liquids and a second end. The tube or tubes may be connected to a heater or heating element (e.g., via a conductive pathway thermally connecting the heater to the tube or tubes. The material comprising the tube or tubes may have a permeability such that a portion of the gas dissolved in the fluid or liquid passes through the material to an external region upon being degassed from a portion of the liquid within the tube or tubes. For such embodiments comprising a degassing element or feature, factors that are known in the art can be used and/or varied vary in order to control the amount of degassing (e.g., pressure and temperature differentials of fluid within the tube or tubes of the dispensing system. In some embodiments, the pressure differential between a fluid and ambient atmospheric pressure can be constant. In some embodiments, the actual gauge pressure differential measured from inside the dispensing system to outside the dispensing system (that is, ambient atmospheric conditions) can be measured and is different from zero. In embodiments, the amount of gas dissolved in a liquid is proportional to the concentration of that gas and the pressure that it is under (amongst other factors). In some embodiments, heating a fluid, such as liquid sample or liquid assay reagent, in a degasser allows for expansion of the fluid and contributes to an increase in pressure between a pump inlet valve, a pump, a degasser tube or loop, and a dispenser valve. In such embodiments, a pressure build up can impact the ability of the heater to degas the liquid which can impacts the relative light unit (RLU) produced by liquid (e.g., mixture of sample and assay reagent(s)).

In some embodiments, immunoassay analyzer or instrument includes a structure to heat a fluid prior to it being added to the reaction vessel. The structure may include a dispenser. The fluid may include a wash fluid or a substrate composition. The wash fluid can be added to particles bound to the bound antibody or antigen. The wash fluid may be added to particles before and/or after the particles are aggregated (for example, paramagnetic particles may be aggregated by being subjected to a magnetic field). The substrate composition is typically added to the particles after they are washed. The fluid may be heated using any suitable structure. Exemplary dispenser used to heat a fluid are described in, for example, U.S. Pat. No. 10,562,021. In some aspects, the structure to heat the fluid may include a dispenser that includes a fluid pathway, a heat source that is in thermal communication with the fluid pathway, and a probe that includes an inlet and an outlet and contains the fluid. In some embodiments, the dispenser preferably dispenses the fluid at desired fluid temperature so as to maintain the desired fluid temperature or desired fluid temperature range at dispense rates, frequencies, and dispense volumes without drawing-back and purging the dispense fluid.

In some embodiments, the immunoassay analyzer or instrument includes a luminometer as described in WO2019060375, WO2020219869, and/or U.S. Pat. No. 11,604,146. For example, the immunoassay analyzer or instrument may include: a reaction vessel chamber; a light passage intersecting the reaction vessel chamber; a cap that, when in a closed configuration, forms a dark chamber by preventing light emitted by external light sources from entering the reaction vessel chamber and from entering the light passage, and, when in an open configuration, provides access to the reaction vessel chamber; and light detector optically coupled to the light passage, the light detector comprising a sensing element for receiving light from the light passage. Additionally or alternatively, the immunoassay analyzer or instrument may include a light detector configured to sense photons emitted from assay reactions over a period of time; an analog circuit configured to provide an analog signal based on the photons emitted from the assay reactions over the period of time; a counter circuit configured to provide a photon count based on the photons emitted from the assay reactions over the period of time; and a luminometer controller configured to: in response to an analog signal value of the analog signal being greater than a predetermined value, determine and report a measurement value of the photons emitted from the assay reactions over the period of time based on the analog signal value of the analog signal and a linear function, wherein the linear function is derived from a relationship established between the analog signal and the photon count. Additionally or alternatively, the immunoassay analyzer or instrument may include a luminometer system, comprising: a photomultiplier tube configured to sense photons emitted from an assay reaction over a period of time; an analog circuit configured to provide an assay analog voltage based on the photons emitted from the assay over the period of time, the analog circuit comprising: a current sensing resistor coupled to convert current from the photomultiplier tube to a voltage; an amplifier configured to amplify the voltage; and a dedicated electrical connection between a terminal of the current sensing resistor and a terminal of the amplifier; a counter circuit configured to provide an assay photon count based on the photons emitted from the assay over the period of time; and a luminometer controller configured to: in response to the assay analog voltage being greater than a predetermined value, calculating a relative light unit value of the photons emitted from the assay over the period of time based on the assay analog voltage and an optimized linear function.

The following examples are provided merely in an effort to more fully describe and illustrate some of the aspects and embodiments described herein. It is to be understood that the particular examples, materials, amounts, and procedures are to be interpreted broadly in accordance with the scope and spirit of the description as set forth herein.

### ILLUSTRATIVE EXAMPLES

All reagents, starting materials, and solvents used in the Examples were purchased from commercial suppliers (such as Sigma Aldrich, St. Louis, MO) and were used without further purification unless otherwise indicated.

### Example 1—Assay Migration Studies on the Beckman Coulter DxI 9000 Access Immunoassay Analyzer

The U.S. FDA's guidance for industry and staff titled “Assay Migration Studies for In Vitro Diagnostic Devices” provides a least burdensome approach for the transfer of previously-approved assays from an existing to a new system. (See, Guidance for Industry and Staff: Assay Migration Studies for In Vitro Diagnostic Devices issued 2013, available at the FDA website). This approach enables use of rigorous analytical performance data in place of full clinical data to implement a cleared product on a new platform. The Beckman Coulter DxI 9000 Access Immunoassay Analyzer includes numerous updates and new features designed to improve laboratory workflows and provide quality results to support patient management. Such elements include improved pipetting capabilities, updated process monitoring, increased throughput, reliability enhancements, and software features focused on the needs of the operator. The analyzer also utilizes a new alkaline phosphatase substrate reagent that provides reduced time-to-result for every test as well as other benefits including improved signal-to-noise and reduced sensitivity to endogenous alkaline phosphatase interference. The existing menu of Beckman Coulter Access reagents is being transferred to this system.

Data herein summarize results from analytical studies described within the assay migration guidance and obtained during verification testing of assays for high sensitivity cardiac troponin I (hsTnI) and alpha-fetoprotein (AFP) on the DxI 9000 Access Immunoassay Analyzer. Analytical studies for quantitative assays were performed as directed by the assay migration guidance to compare performance of the Access hsTnI and Access AFP assays across the existing Access 2 and new DxI 9000 immunoassay analyzer systems.

### Methods

### Reproducibility

A multi-site study was performed to evaluate the reproducibility of the Access hsTnI and Access AFP assays on the DxI 9000 and Access 2 immunoassay analyzers using a protocol based on CLSI EP05-A3. (See, CLSI. Evaluation of Precision of Quantitative Measurement Procedures; Approved Guideline—Third Edition. CLSI document EP05-A3. Wayne, PA: Clinical and Laboratory Standards Institute; 2014.).

Reproducibility was evaluated on three DxI 9000 and three Access 2 immunoassay analyzers across three external clinical laboratories. Both serum and lithium heparin plasma samples spanning the range of the assay were measured for hsTnI, while serum samples were tested for AFP. Samples were tested in replicates of three per run with two runs per day over five days on each of the three immunoassay analyzers across the three testing sites. Reproducibility was evaluated using three different reagent pack lots and one calibrator lot. QC were tested daily.

### Comparison Study

A method comparison study was completed to compare the Access hsTnI assay on an DxI 9000 immunoassay analyzer to the Access hsTnI assay on the Access 2 immunoassay analyzer for both serum and plasma sample types. A method comparison study was completed to compare the Access AFP assay on DxI 9000 immunoassay analyzer to the Access AFP assay on the Access 2 immunoassay analyzer for serum. Each study used a protocol based on CLSI EP09C-ED3 and the Assay Migration guidance. (Sec, CLSI. Measurement Procedure Comparison and Bias Estimation Using Patient Samples—Third Edition. CLSI document EP09c, 3rd Ed. Wayne, PA: Clinical and Laboratory Standards Institute; 2018; and FDA Migration Guidance cited above) Method comparison studies were performed on three DxI 9000 immunoassay analyzers and three Access 2 immunoassay analyzers at three external laboratories.

>240 serum samples and >180 lithium heparin plasma samples containing hsTnI concentrations spanning the analytical measuring range of the assay were tested. >200 serum samples containing AFP concentrations spanning the analytical measuring range of the assay were tested. All samples were tested in replicates of one on each of three reagents lots across the three external sites. Each reagent lot was tested at different sites on separate DxI 9000/Access 2 immunoassay analyzer pairs. QC were tested daily.

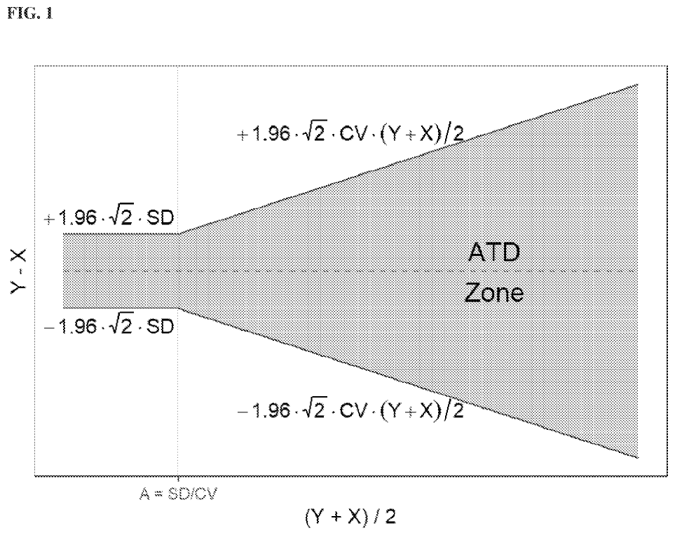

The Assay Migration guidance document cited above provides instructions to calculate an “allowable total difference” or ATD zone, for which 95% of individual sample differences are expected to fall (see FIG. 1).

### Imprecision

Studies were performed to assess the imprecision of the Access hsTnI assay and the Access AFP assay on the DxI 9000 immunoassay analyzer using a protocol based on CLSI EP05-A3. (See, CLSI. Evaluation of Precision of Quantitative Measurement Procedures; Approved Guideline—Third Edition. CLSI document EP05-A3. Wayne, PA: Clinical and Laboratory Standards Institute; 2014.).

Each study was run on three DxI 9000 immunoassay analyzers, three reagent lots and three calibrator lots. Both serum and lithium heparin plasma samples spanning the range of the assay were measured for hsTnI, while serum samples were tested for AFP. Each sample was tested in replicates of two per run for hsTnI and three per run for AFP. Two runs per day were completed over 20 days on each immunoassay analyzer and reagent lot combination. Three commercial quality controls were run in duplicate for each assay on each day to verify the system was in control.

### Detection Capability

Studies were performed to determine the Limit of Blank (LoB), Limit of Detection (LoD), and Limit of Quantitation (LoQ) for the Access hsTnI assay and Access AFP assay using a protocol based on CLSI EP17-A2. (See, CLSI. Evaluation of Detection Capability for Clinical Laboratory Measurement Procedures: Approved Guideline—Second Edition. CLSI document EP17-A2. Wayne, PA: Clinical and Laboratory Standards Institute; 2012.).

For the estimation of LoB, three DxI 9000 immunoassay analyzers were used in the study design with three reagent lots and one calibrator lot. Four S0 calibrator preparations for each respective assay were used for the LoB determination. Blank samples were tested over three days one run per day, five replicates per run, for each pack lot.

For estimation of LoD and LoQ, three DxI 9000 immunoassay analyzer were used in the study design with three reagent lots and one calibrator lot. Both serum and lithium heparin plasma samples containing low levels of analyte were measured for hsTnI, while serum was tested for AFP. Samples were tested in replicates of nine per run with one run per day and five total days on each pack lot and immunoassay analyzer. This design resulted in a minimum of 40 replicates for each sample on each pack lot tested.

Three quality controls were run in replicates of two for each assay on each day to verify the systems were in control.

In some cases, the analyte detected by the immunoassay comprises AFP. The LoB for the AFP immunoassay can be at least 0.05, 0.06, 0.07, 0.08, 0.09, 0.1, 0.11, 0.12, 0.13, or 0.14 ng/ml and/or up to 0.1, 0.11, 0.12, 0.13, 0.14, 0.15, 0.16, 0.17, 0.18, 0.19, 0.2, 0.25, or 0.3 ng/mL. The LoB for the AFT immunoassay can be in a range from about 0.05 to about 0.3, from about 0.06 to about 0.25, from about 0.07 to about 0.2, or from about 0.08 to about 0.15 ng/ml. The LoD for the AFP immunoassay can be at least 0.1, 0.12, 0.14, 0.16, 0.18, 0.2, or 0.22 ng/ml, and/or up to 0.2, 0.21, 0.22, 0.23, 0.24, 0.25, 0.26, 0.28, or 0.3 ng/mL. The LoD for the AFT immunoassay can be in a range from about 0.1 to about 0.3, from about 0.12 to about 0.28, from about 0.14 to about 0.26, from about 0.16 to about 0.24, from about 0.18 to about 0.24, from about 0.2 to about 0.24, or from about 0.22 to about 0.24 ng/mL. The LoQ for the AFP immunoassay can be at least 0.08, 0.09, 1.1, 0.11, 0.12, or 0.13 ng/mL, and/or up to 0.14, 0.15, 0.16, 0.17, 0.18, 0.19, 0.2, 0.21, 0.22, 0.24, 0.26, 0.28, or 0.3 ng/mL. The LoQ for the AFP immunoassay can be in a range from about 0.08 to about 0.3, from about 0.1 to about 0.25, from about 0.12 to about 0.2, or from about 0.14 to about 0.18 ng/mL. In some cases, the AFP immunoassay has a LoB in a range from about 0.05 to about 0.3, from about 0.06 to about 0.25, from about 0.07 to about 0.2, or from about 0.08 to about 0.15 ng/mL, a LoD in a range from about 0.1 to about 0.3, from about 0.12 to about 0.28, from about 0.14 to about 0.26, from about 0.16 to about 0.24, from about 0.18 to about 0.24, from about 0.2 to about 0.24, or from about 0.22 to about 0.24 ng/ml, and a LoQ in a range from about 0.08 to about 0.3, from about 0.1 to about 0.25, from about 0.12 to about 0.2, or from about 0.14 to about 0.18 ng/mL.

In some cases, the analyte detected by the immunoassay comprises TSH. The LoB for the TSH immunoassay can be at least 0.0005, 0.0006, 0.0007, 0.0008, 0.0009, 0.001, 0.0011, 0.0012, 0.0013, 0.0014, or 0.0015 uIU/mL, and/or up to 0.0016, 0.0017, 0.0018, 0.0019, 0.002, 0.0021, 0.0022, 0.0024, 0.0026, 0.0028, or 0.003 uIU/mL. The LoB for the TSH immunoassay can be in a range from about 0.0005 to about 0.003, from about 0.0006 to about 0.0025, from about 0.0008 to about 0.0022, from about 0.001 to about 0.002, from about 0.0012 to about 0.0018, or from 0.0014 to about 0.0016 uIU/mL. The LoD for the TSH immunoassay can be at least 0.001, 0.0012, 0.0014, 0.0016, 0.0018, 0.002, 0.0022, or 0.0024 uIU/mL, and/or up to 0.0026, 0.0028, 0.003, 0.0032, 0.0034, 0.0036, 0.0038, or 0.004 uIU/mL. The LoD for the TSH immunoassay can be in a range from about 0.001 to about 0.004, from about 0.0015 to about 0.0035, from about 0.002 to about 0.003, or from about 0.0022 to about 0.0028 uIU/mL. The LoQ for the TSH immunoassay can be at least 0.0003, 0.0004, 0.0005, 0.0006, or 0.0007 uIU/mL, and/or up to 0.0008, 0.0009, 0.001, 0.0011, 0.0012, 0.0013, 0.0014, or 0.0015 uIU/mL. The LoQ for the TSH immunoassay can be in a range from about 0.0003 to about 0.0015, from about 0.0005 to about 0.0012, or from about 0.0008 to about 0.001. In some cases, the TSH immunoassay has a LoB in a range from about 0.0005 to about 0.003, from about 0.0006 to about 0.0025, from about 0.0008 to about 0.0022, from about 0.001 to about 0.002, from about 0.0012 to about 0.0018, or from 0.0014 to about 0.0016 uIU/mL, a LoD in a range from about 0.001 to about 0.004, from about 0.0015 to about 0.0035, from about 0.002 to about 0.003, or from about 0.0022 to about 0.0028 uIU/mL, and a LoQ in a range from about 0.0003 to about 0.0015, from about 0.0005 to about 0.0012, or from about 0.0008 to about 0.001.

In some cases, the analyte detected by the immunoassay comprises hCG. The LoB for the hCG immunoassay can be at least 0.0, 0.0005, 0.001, 0.01, or 0.05 mIU/mL, and/or up to 0.06, 0.07, 0.08, 0.09, 0.1, 0.15, or 0.2 mIU/mL. The LoB for the hCG immunoassay can be in a range from about 0.0 to about 0.2, from about 0.0005 to about 0.15, from about 0.001 to about 0.15, from about 0.01 to about 0.1, or from about 0.05 to about 0.1 mIU/mL. The LoD for the hCG immunoassay can be at least 0.001, 0.005, 0.01, or 0.05 mIU/mL, and/or up to 0.1, 0.12, 0.14, 0.16, 0.18, 0.2, 0.25, or 0.3 mIU/mL. The LoD for the hCG immunoassay can be in a range from about 0.001 to about 0.3, from about 0.005 to about 0.3, from about 0.01 to about 0.25, from about 0.1 to about 0.25, or from about 0.15 to about 0.2 mIU/mL. The LoQ for the hCG immunoassay can be at least 0.001, 0.005, 0.01, 0.05, or 0.1 mIU/mL, and/or up to 0.12, 0.14, 0.16, 0.18, 0.2, 0.22, 0.24, 0.26, 0.28, or 0.3 mIU/mL. The LoQ for the hCG immunoassay can be in a range from about 0.001 to about 0.3, from about 0.01 to about 0.25, from about 0.05 to about 0.25, from 0.1 to about 0.2, or from 0.12 to about 0.18 mIU/mL. In some cases, hCG immunoassay has a LoB in a range from about 0.0 to about 0.2, from about 0.0005 to about 0.15, from about 0.001 to about 0.15, from about 0.01 to about 0.1, or from about 0.05 to about 0.1 mIU/mL, a LoD in a range from about 0.001 to about 0.3, from about 0.005 to about 0.3, from about 0.01 to about 0.25, from about 0.1 to about 0.25, or from about 0.15 to about 0.2 mIU/mL, and a LoQ in a range from about 0.001 to about 0.3, from about 0.01 to about 0.25, from about 0.05 to about 0.25, from 0.1 to about 0.2, or from 0.12 to about 0.18 mIU/mL.

In some cases, the analyte detected by the immunoassay comprises free T4. The LoB for the free T4 immunoassay can be at least 0.001, 0.005, 0.01, 0.05, 0.1, 0.12, or 0.14 ng/ml, and/or up to 0.16, 0.18, 0.2, 0.22, or 0.24 ng/mL. The LoB for the free T4 immunoassay can be in a range from about 0.001 to about 0.3, from about 0.01 to about 0.25, from about 0.1 to about 0.2, or from about 0.15 to about 0.2 ng/ml.

### Linearity

Studies were performed to assess the linearity of the Access hsTnI assay and the Access AFP assay on the DxI 9000 immunoassay analyzer based on CLSI EP06-Ed2. (See, CLSI. Evaluation of Linearity of Quantitative Measurement Procedures. CLSI document EP06-Ed2. Wayne, PA: Clinical and Laboratory Standards Institute; 2020.).

Samples covering the full analytical measuring range of each assay were used for the linearity determination. Both serum and lithium heparin plasma sample types were evaluated independently for hsTnI, while serum was tested for AFP. A native sample containing a concentration at the low end of the measuring interval was obtained. A high sample was prepared by spiking antigen into a low sample until a concentration above the highest commercial calibrator was achieved. In addition to the high and low concentration samples, seven mixtures were tested in this study. These samples were prepared independently by using incrementally larger proportions of the high sample diluted with the low sample, to achieve concentrations that covered the range of the assay. The low sample was run in replicates of eight, and all other samples were run in replicates of four. This study was run on one DxI 9000 immunoassay analyzer, using three reagent lots and one calibrator lot. Three quality controls were run in replicates of two for each assay on each day to verify the system was in control.

### Results

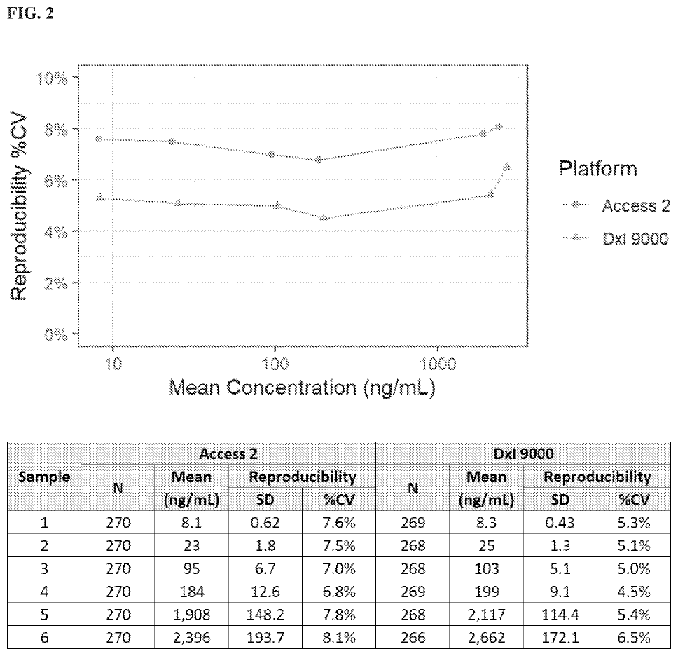

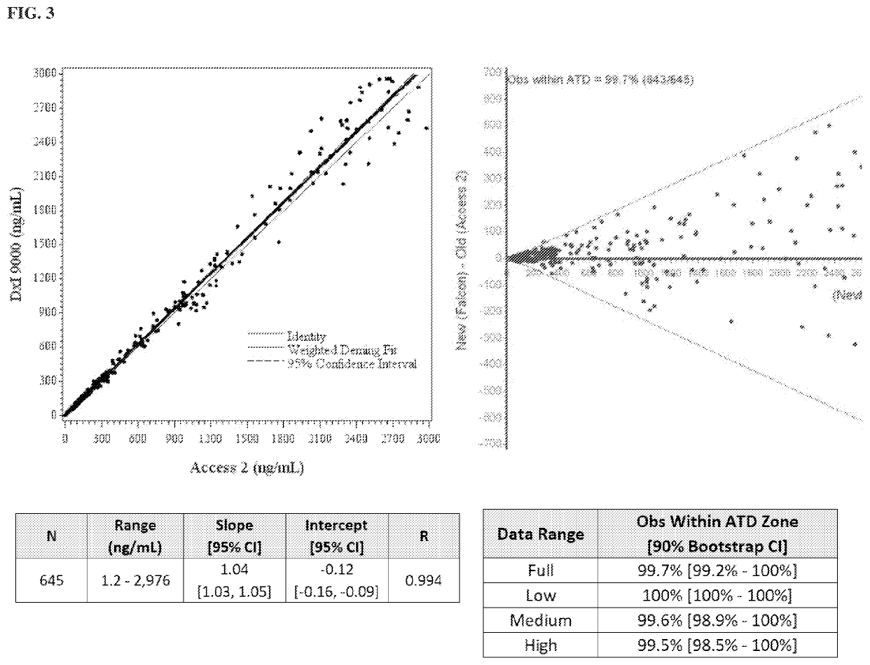

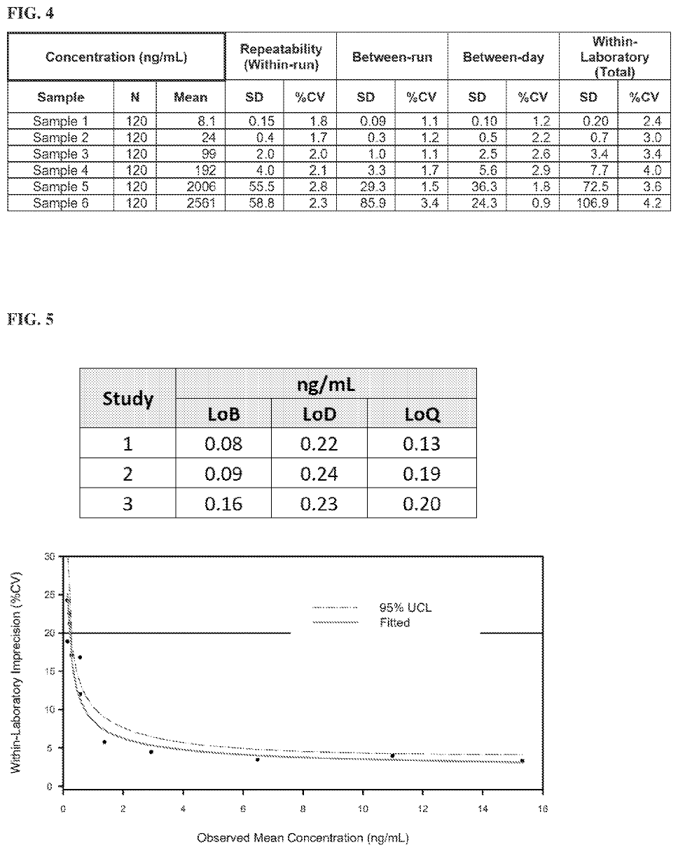

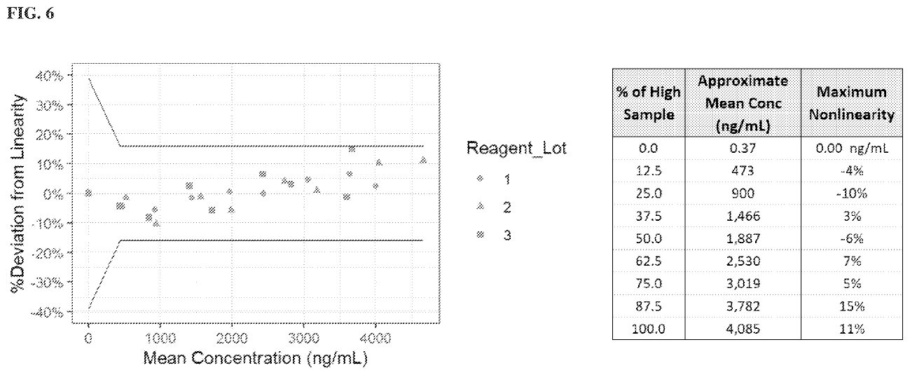

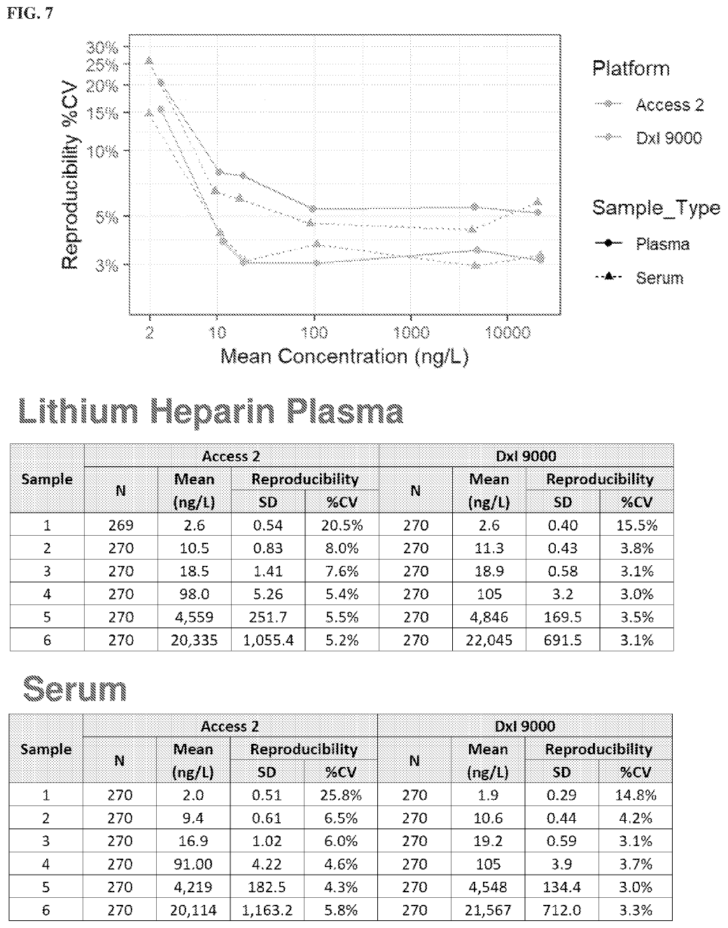

Results for Access AFP (for alpha-fetoprotein) are shown in FIG. 2-FIG. 6 and are described in the descriptions thereof.

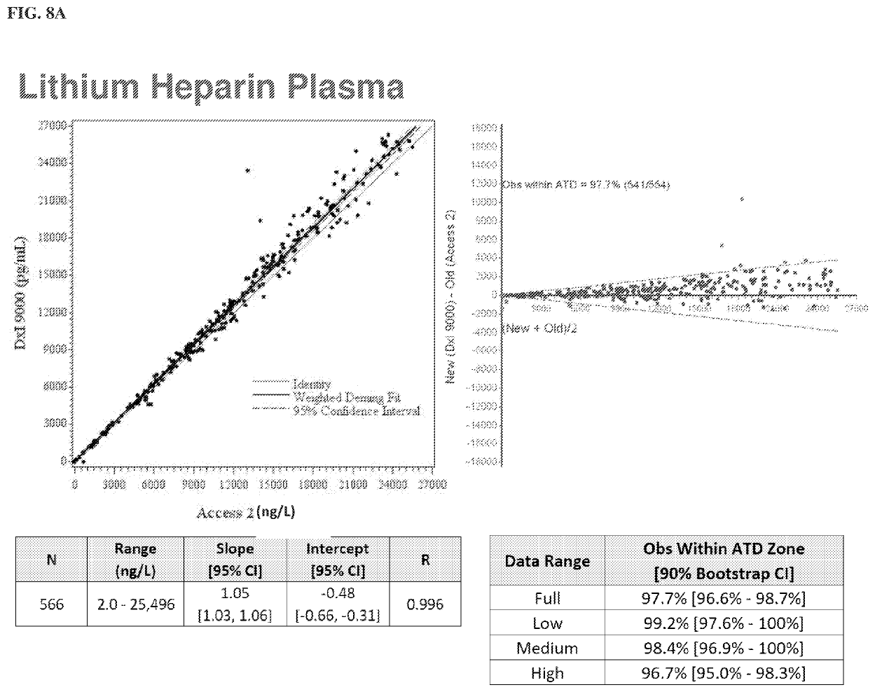

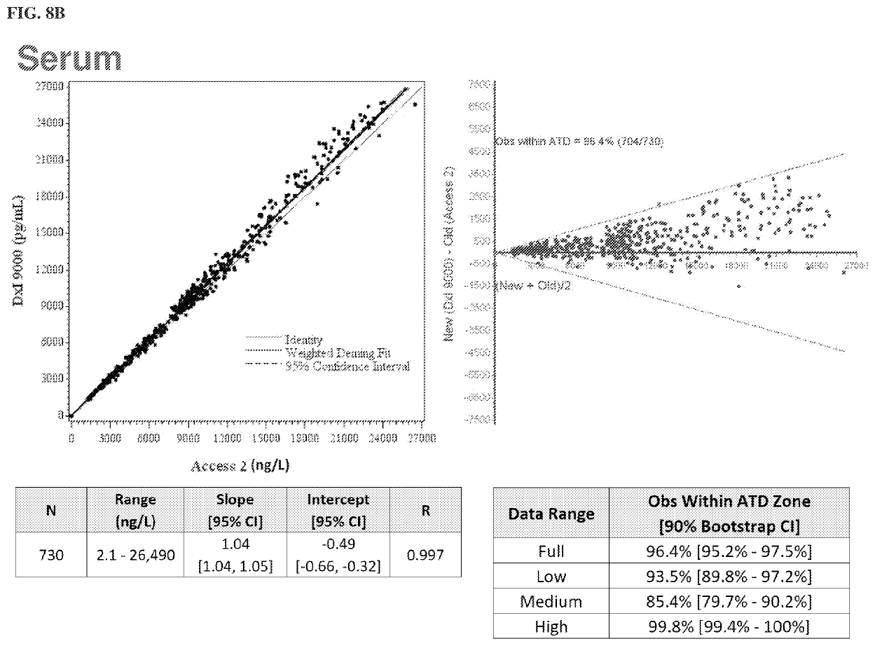

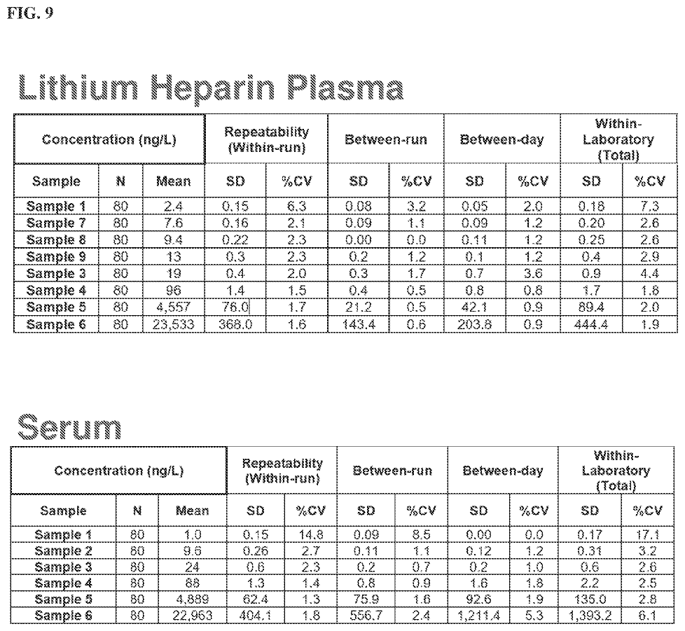

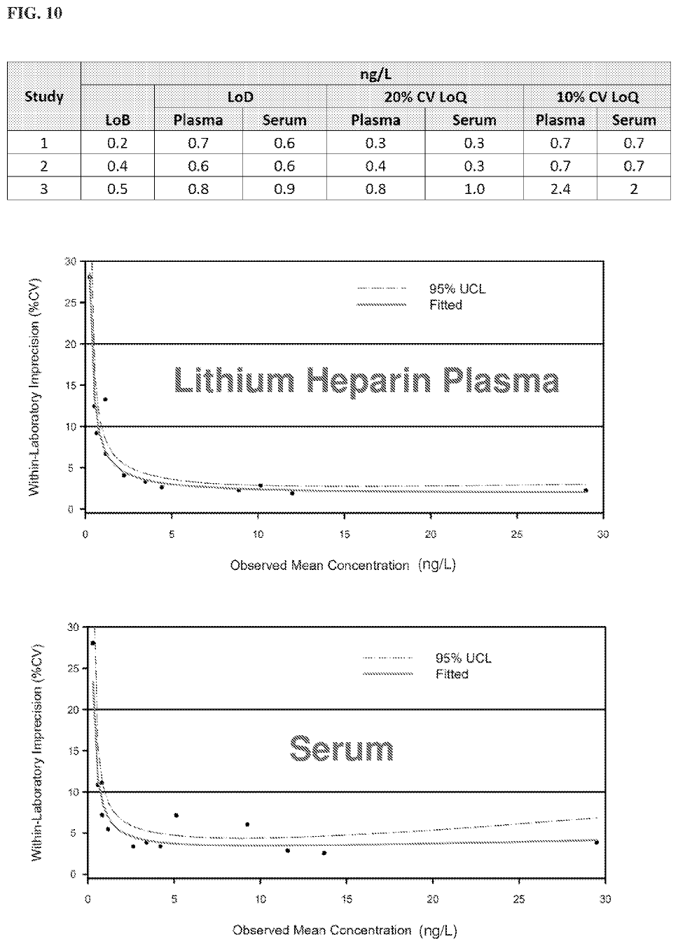

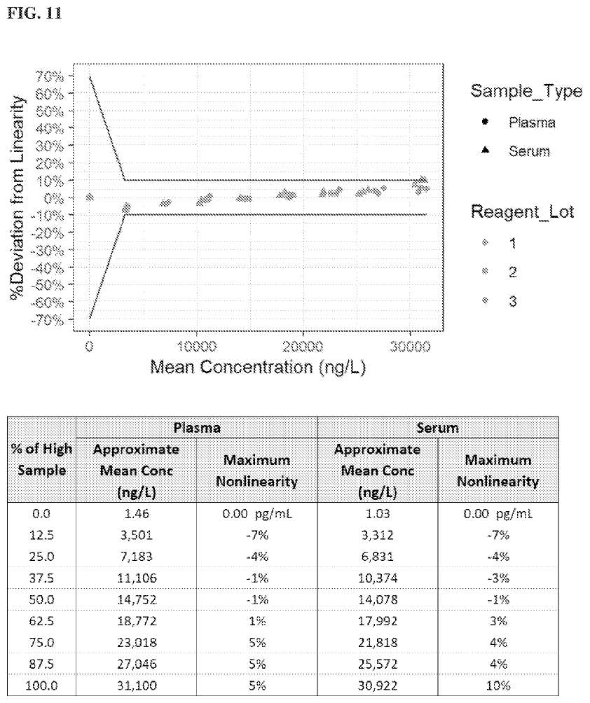

Results for Access hsTnI (for cardiac troponin) are shown in FIG. 7-FIG. 11 and are described in the descriptions thereof.

### Conclusions

Individual assay data generated for the Access hsTnI and Access AFP assays on the DxI 9000 Access Immunoassay Analyzer including accuracy, imprecision, detection capability, and linearity met US FDA assay migration guidance study design criteria and demonstrate acceptable assay performance.

### Example 2—Assay Performance Using Improved Compositions

Alkaline phosphatase (ALP) was coated onto paramagnetic particles (PMPs) and then stored in various diluents (pH 6, 7, or 8) for 0, 1 and 3 days at 37° C. Incubation of ALP coated onto PMPs for 3 days at 37° C. has previously been demonstrated to achieve nearly identical results to incubation of ALP coated onto PMPs for 6 months at 4° C.

The activity of the ALP-coated particles was then assessed on a DxI 800 immunoassay analyzer (Beckman Coulter, Inc., Brea, CA). The ALP-coated particles were loaded into reagent packs and subsequently loaded onto the DxI 800 immunoassay analyzer. The system washed the particles to eliminate the storage solution and then the activity was assessed with LUMI-PHOS PRO (Lumigen, Inc., Southfield, MI).

Results after 3 days at 37° C. are shown in FIG. 14. As seen in FIG. 14, although a broad range of concentrations of magnesium and zinc allow for ALP stability over 3 days at 37° C. at pH 8, a much narrower range of concentrations of magnesium and zinc allow for ALP stability over 3 days at 37° C. at pH 7, and a very narrow range of concentrations of magnesium and zinc allow for optimal ALP stability and activity over 3 days at 37° C. at pH 6.

Table 1 details the estimated apparent dissociation constants between metal species and the M3 site of bovine intestinal ALP as a function of pH.

TABLE 1

| | | |
|---|---|---|
| pH | K_{App (Mg)} | K_{App (Zn)} |

| | | | |
|---|---|---|---|
| 6 | 10 | mM | 100 uM |
| 7 | 2 | mM | 100 uM |
| 8 | <<2 | mM | >100 uM |

### Example 3—Targeted Antigen Assays Using Improved Compositions

This Example describes the optimization of free magnesium and zinc ion concentrations in compositions in which ALP is conjugated to an antibody or an antigen for use in a specific immunoassay. Optimal ALP activity (as measured by relative light units (RLU)) was balanced with other features that affect assay performance including antibody- or antigen-conjugate stability, pH, buffers, albumin (for example, BSA) concentration, and dose recovery to determine the final amounts of magnesium and zinc selected.

### Example 3A—AFP

This Example describes an immunoassay for the detection of alpha-fetoprotein (AFP) in a sample. The Example further describes an exemplary composition for use in the immunoassay that includes a mouse monoclonal anti-AFP antibody conjugated to ALP.

The assay to detect AFP is a two-site immunoenzymatic (“sandwich”) assay. A sample is added to a reaction vessel with mouse monoclonal anti-AFP-alkaline phosphatase conjugate, and paramagnetic particles coated with a second mouse monoclonal anti-AFP antibody. The AFP in the sample binds to the immobilized monoclonal anti-AFP on the solid phase while, at the same time, the monoclonal anti-AFP-alkaline phosphatase conjugate reacts with different antigenic sites on the sample AFP.

10 μL of serum samples (or alternatively amniotic fluid samples) containing a range of concentrations of AFP were loaded on a DxI 9000 immunoassay analyzer using disposable pipette tips. The immunoassay analyzer aliquoted diluted samples into individual reaction vessels (RVs).

To perform the immunoenzymatic assay, the immunoassay analyzer dispensed to each RV 52 μg paramagnetic microparticles (PMPs) coated with anti-AFP antibody. The immunoassay analyzer also dispensed to each RV anti-AFP antibodies conjugated to ALP. The contents of the RV tube were mixed and incubated at 37° C. for about 5 min. After incubation in the RV, materials bound to the PMPs were held in a magnetic field while unbound materials are washed away. Then, 200 μL of, LUMI-PHOS PRO, was added to each RV tube. Light generated by the reaction was measured with a luminometer. The light production is directly proportional to the concentration of AFP in the sample. The amount of analyte in the sample was determined from a stored, multi-point calibration curve.

The composition including the mouse monoclonal anti-AFP antibody conjugated to ALP had a pH of 6. The composition was optimized to include 15 mM MgCl₂ and 100 μM ZnCl₂, resulting in an estimated concentration of free Mg²⁺ of 15 mM and an estimated concentration of free Zn²⁺ of 85 μM. The ratio of free [Mg²⁺]/free [Zn²⁺] is 176. Without wishing to be bound by theory, it is believed that additional ZnCl₂ to achieve was required a free Zn²⁺ in the desired range (see Example 1) because the composition including the mouse monoclonal anti-AFP antibody conjugated to ALP includes albumin which complexes to zinc ions. The estimated concentration of free Zn²⁺ is based on 1:1 binding between BSA and Zn and does not include the weaker interaction with PO₄.

### Example 3B—βhCG

This Example describes an immunoassay for the detection of beta human chorionic gonadotropin (βhCG) in a sample. The Example further describes an exemplary composition for use in the immunoassay that includes a rabbit anti-βhCG antibody conjugated to ALP.

The assay to detect βhCG is a two-site immunoenzymatic (“sandwich”) assay. A sample is added to a reaction vessel along with a citrate buffer. After an initial incubation, rabbit anti-βhCG alkaline phosphatase conjugate and paramagnetic particles coated with goat anti-mouse IgG: mouse monoclonal anti-βhCG complexes are added. The hCG binds to the immobilized monoclonal anti-βhCG on the solid phase while, at the same time, the rabbit anti-βhCG alkaline phosphatase conjugate reacts with different antigenic sites on the hCG.

13 μL of serum samples (or alternatively, plasma samples) containing a range of concentrations of βhCG were loaded on a DxI 9000 immunoassay analyzer using disposable pipette tips. The immunoassay analyzer aliquoted samples into individual reaction vessels (RVs).

To perform the immunoenzymatic assay, the immunoassay analyzer dispensed to each RV 50 μg of paramagnetic microparticles (PMPs) coated with anti-βhCG antibody. The immunoassay analyzer also dispensed to each tube βhCG antibodies conjugated to ALP. The contents of the RV tube were mixed and incubated at 37° C. for about 6 min. After incubation in the RV, materials bound to PMPs were held in a magnetic field while unbound materials are washed away. Then, 200 μL of LUMI-PHOS PRO, was added to each RV tube. Light generated by the reaction was measured with a luminometer. The light production is directly proportional to the concentration of total βhCG in the sample. The amount of analyte in the sample was determined from a stored, multi-point calibration curve.

The composition including the rabbit anti-βhCG antibody conjugated to ALP had a pH of 6. The composition was optimized to include 32 mM MgCl₂ and 50 μM ZnCl₂, resulting in an estimated concentration of free Mg²⁺ of 32 mM and an estimated concentration of free Zn²⁺ of 35 μM. The ratio of free [Mg²⁺]/free [Zn²⁺] is 914. Without wishing to be bound by theory, it is believed that additional ZnCl₂ was required because the composition including the rabbit anti-βhCG antibody conjugated to ALP includes bovine serum albumin (BSA) which complexes to zinc ions. The estimated concentration of free Zn²⁺ is based on binding between albumin and Zn and does not include the weaker/more complex interaction with ACES.

### Example 3C—Free T4

This Example describes an immunoassay for the detection of thyroxine (T4). The Example further describes an exemplary composition for use in the immunoassay that includes a triiodothyronine (T3) antigen conjugated to ALP.

The assay to detect (free) T4 is a two-step enzyme immunoassay. Monoclonal anti-thyroxine (T4) antibody coupled to biotin, sample, buffered protein solution, and streptavidin-coated solid phase are added to the reaction vessel. During this first incubation the anti-T4 antibody coupled to biotin binds to the solid phase and the free T4 in the sample. After incubation in a reaction vessel, materials bound to the solid phase are held in a magnetic field while unbound materials are washed away. Next, buffered protein solution and triiodothyronine (T3)-ALP conjugate are added to the reaction vessel. The T3-ALP conjugate binds to the vacant anti-T4 antibody binding sites.

30 μL serum samples (or alternatively, plasma samples) containing a range of concentrations of (free) T4 were loaded on DxI 9000 immunoassay analyzers using disposable pipette tips. The immunoassay analyzer aliquoted samples into individual reaction vessels (RVs).

To perform the immunoassay, the immunoassay analyzer dispensed to each RV anti-T4 antibody coupled to biotin, 18 μg streptavidin-coated paramagnetic microparticles (PMPs), and triiodothyronine (T3) conjugated to ALP. The contents of the RV tube were mixed and incubated at 37° C. for about 13 min. After incubation in the RV, materials bound to the PMPs were held in a magnetic field while unbound materials were washed away. Then, 200 μL of LUMI-PHOS PRO was added to each RV tube. Light generated by the reaction was measured with a luminometer. The light production is inversely proportional to the concentration of free T4 in the sample. The amount of analyte in the sample was determined from a stored, multi-point calibration curve.

The composition including the T3 antigen conjugated to ALP had a pH of 6. The composition was optimized to include 2 mM MgCl₂ and 10 μM ZnCl₂, resulting in an estimated concentration of free Mg²⁺ of 2 mM and an estimated concentration of free Zn²⁺ of 10 μM. The ratio of free [Mg²⁺]/free [Zn²⁺] is 200. Without wishing to be bound by theory, it is believed that additional ZnCl₂ was required because the composition including the T3 antigen conjugated to ALP includes ovalbumin which complexes to zinc ions.

### Example 3D—cTnI

This Example describes an immunoassay for the detection of cardiac troponin 1 (cTnI). The Example further describes an exemplary composition for use in the immunoassay that includes a monoclonal anti-cTnI antibody conjugated to ALP.

The assay to detect cTnI is a two-site immunoenzymatic (“sandwich”) assay. Monoclonal anti-cTnI antibody conjugated to alkaline phosphatase is added to a reaction vessel along with a surfactant-containing buffer and sample. After a short incubation, paramagnetic particles coated with monoclonal anti-cTnI antibody are added. The human cTnI binds to the anti-cTnI antibody on the solid phase, while the anti-cTnI antibody-ALP conjugate reacts with different antigenic sites on the cTnI molecules.

50 μL serum samples (or alternatively plasma samples) containing a range of concentrations of cTnI were loaded on DxI 9000 immunoassay analyzers using disposable pipette tips. The immunoassay analyzer aliquoted samples into individual reaction vessels (RVs).

To perform the immunoenzymatic assay, the immunoassay analyzer dispensed to each RV a solution of 75 μg paramagnetic microparticles (PMPs) coated with anti-cTnI-antibody. The immunoassay analyzer also dispensed to each tube anti-cTnI antibodies conjugated to ALP. The contents of the RV tube were mixed and incubated at 37° C. for about 5 min. After incubation in the RV, materials bound to the PMPs are held in a magnetic field while unbound materials are washed away. Then, 200 μL of LUMI-PHOS PRO was added to each RV tube. Light generated by the reaction was measured with a luminometer. The light production is directly proportional to the concentration of cTnI in the sample. The amount of analyte in the sample was determined from a stored, multi-point calibration curve.

The composition including the monoclonal anti-cTnI antibody conjugated to ALP had a pH of 6. The composition was optimized to include 35 mM MgCl₂ and 250 μM ZnCl₂, resulting in an estimated concentration of free Mg²⁺ of 35 mM and an estimated concentration of free Zn²⁺ of 175 μM. Without wishing to be bound by theory, it is believed that additional ZnCl₂ was required because the composition including the monoclonal anti-cTnI antibody conjugated to ALP includes bovine serum albumin (BSA) and ACES buffer, each of which complex to zinc ions.

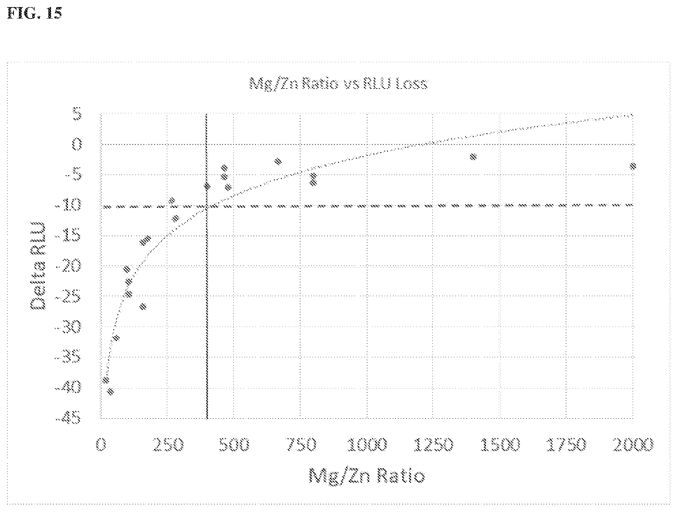

The ratio of free [Mg²⁺]/free [Zn²⁺] is 200. As shown in FIG. 15, the Mg/Zn ratio selected was not optimized for RLU loss alone but still provides acceptable performance.

### Example 3E—TSH

This Example describes an immunoassay for use in the detection of human thyroid-stimulating hormone (hTSH) in a sample. The Example further describes an exemplary composition for use in the immunoassay that includes a mouse anti-hTSH antibody conjugated to ALP.

The assay to detect hTSH is a two-site immunoenzymatic (“sandwich”) assay. A sample is added to a reaction vessel with mouse anti-hTSH-alkaline phosphatase conjugate, buffered protein solution and paramagnetic particles coated with immobilized mouse monoclonal anti-hTSH antibody. The hTSH binds to the immobilized monoclonal anti-hTSH antibody on the solid phase while the mouse anti-hTSH-alkaline phosphatase conjugate reacts with a different antigenic site on the hTSH.

25 μL serum samples (or alternatively, plasma samples) containing a range of concentrations of hTSH were loaded on DxI 9000 immunoassay analyzers using disposable pipette tips. The immunoassay analyzer aliquoted samples into individual reaction vessels (RVs).

The immunoassay analyzer dispensed to each tube 13 μg of paramagnetic microparticles (PMPs) coated with anti-hTSH antibody. The immunoassay analyzer also dispensed to each tube anti-hTSH antibodies conjugated to ALP. The contents of the RV tube were mixed and incubated at 37° C. for about 14 min. After incubation in the RV, materials bound to the PMPs were held in a magnetic field while unbound materials are washed away. Then, 200 μL of LUMI-PHOS PRO was added to each RV tube. Light generated by the reaction was measured with a luminometer. The light production is directly proportional to the concentration of hTSH in the sample. The amount of analyte in the sample was determined from a stored, multi-point calibration curve.

The composition including the mouse anti-hTSH antibody conjugated to ALP has a pH of 6. The composition was optimized to include 8 mM MgCl₂ and 25 μM ZnCl₂, resulting in an estimated concentration of free Mg²⁺ of 8 mM and an estimated concentration of free Zn²⁺ of 10 μM. Without wishing to be bound by theory, it is believed that additional ZnCl₂ was required because the composition including the mouse anti-hTSH antibody conjugated to ALP includes bovine serum albumin (BSA) and ACES buffer, each of which complex to zinc ions. The estimated concentration of free Zn²⁺ is based on 1:1 binding between BSA and Zn and does not include the weaker/more complex interaction with ACES. The ratio of free [Mg²⁺ ]/free [Zn²⁺] is 800.

### Example 4—Dose Bias Assay

To determine if ALP activity is maintained prior to the addition of the ALP to the substrate, a dose bias assay was performed. The RLU response activity of LUMI-PHOS 530 (Lumigen, Southfield, MI) and LUMI-PHOS PRO (Lumigen, Southfield, MI) were measured against a known concentration of analyte, using an immunoassay reagent composition including ALP but in which the Mg/Zn ratio was not formulated to optimize ALP activity under all conditions. Then the immunoassay reagent composition was stored at 4° C. for 6 months (from April to October). RLU response activity of LUMI-PHOS 530 and LUMI-PHOS PRO were again used to detect analyte.

As shown in FIG. 13, in an assay that includes LUMI-PHOS 530, little or no dose bias is observed. In an assay that includes LUMI-PHOS PRO, a dose bias was observed as a result of loss of ALP activity during storage of the enzyme in the immunoassay reagent composition.

### EXEMPLARY ASPECTS

Some exemplary aspects relate to a method comprising:
measuring serum or plasma concentration of a target analyte in a biological sample;
wherein the measuring comprises detecting the target analyte using an antibody to the target analyte or an antigen associated with the target analyte; and
wherein the method further comprises measuring the concentration of target analyte using a substrate formulation comprising a chemiluminescent compound.

Some exemplary aspects relate to methods comprising any one or more of A1-A12 and/or B1-B10 as follows:
A1. A method comprising:
   measuring serum or plasma concentration of cardiac troponin I (cTnI) in a blood sample;
   wherein the method comprises detecting cTnI using an anti-cTnI antibody, and
   wherein the method further comprises measuring the concentration of cTnI using a substrate formulation comprising a chemiluminescent compound.
A2. The method of A1, wherein the method comprises measuring cTnI in an apparatus comprising a dispense control system, an image capture device, or camera unit or a combination thereof.
A3. The method of A1 or A2, wherein the method further comprises comparing the cTnI concentrations to a calibration curve.
A4. The method of any of A1 to A3, wherein the chemiluminescent compound comprises formula I or a salt thereof:

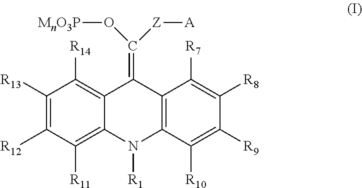

wherein
   A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
   R₁ is selected from the group consisting of C₅₋₁₄aryl, C₁₋₆ alkyl, C₁₋₆ haloalkyl, and C₅₋₁₄ aralkyl groups;
   R₇-R₁₄ are independently H, C₁₋₆ alkoxy, halo, C₁₋₄alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
   R₁₅ is C₁₋₆ alkyl;
   each M is independently selected from the group consisting of H, an alkali metal, alkaline earth metal, transition metal, ammonium, phosphonium, organic amine salt, and an amino acid salt;
   Z is O or S; and
   n is 0, 1, or 2;
A5. The method of any of A1 to A4, wherein the substrate formulation further comprises:
   a cationic aromatic compound (CAC);
   a background reducing agent; or
   an ether-linked nonionic surfactant that does not contain a carboxylate ester group; or a hydrophilic polymer; or
   a combination thereof.
A6. The method of any of A1 to A5, wherein the substrate formulation comprises
   a) 0.01 mM-50 mM of a chemiluminescent compound of formula I or a salt thereof;
   b) 0.01-200 μM of a cationic aromatic compound (CAC);
   c) 1 μM-10 mM of a background reducing agent; and
   d) 0.05-20 g/L of an ether-linked nonionic surfactant that does not contain a carboxylate ester group; or a hydrophilic polymer.
A7. The method of any of A1 to A6, wherein the measurement of cTnI exhibits a coefficient of variation of less than 4% for concentrations of cTnI in plasma greater than 10 ng/ml.
A8. The method of any of A1 to A7, wherein the measurement of cTnI exhibits a standard deviation (SD) of less than 0.5 ng/ml for concentrations of cTnI in plasma less than 10 ng/mL.
A9. The method of any of A1 to A6, wherein the measurement of cTnI exhibits a coefficient of variation of less than 4% for concentrations of cTnI in serum greater than 15 ng/mL.
A10. The method of any of A1 to A6 or A9, wherein the measurement of cTnI exhibits a standard deviation (SD) of less than 0.5 ng/mL for concentrations of cTnI in serum less than 15 ng/ml.
A11. The method of any of A7 to A10, where the reproducibility conditions of measurement include at least between-site and between-lot variance components.
A12. The method of any of A7 to A10, where the reproducibility conditions of measurement include between-site, between-lot, between-day, between-run, and within-run variance components.
B1. A method comprising:
   measuring serum or plasma concentration of alpha-fetoprotein (AFP) in a blood sample;
   wherein the method comprises detecting AFP using an anti-AFP antibody, and
   wherein the method further comprises measuring the concentration of AFP using a substrate formulation comprising a chemiluminescent compound.
B2. The method of B1, wherein the method comprises measuring AFP in an apparatus comprising a dispense control system, an image capture device, or camera unit or a combination thereof.
B3. The method of B1 or B2, wherein the method further comprises comparing the cTnI concentrations to a calibration curve.
B4. The method of any of B1 to B3, wherein the chemiluminescent compound comprises formula I or a salt thereof:

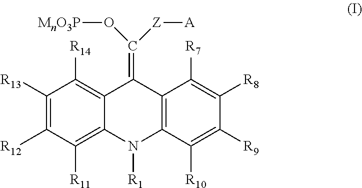

wherein
   A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
   R₁ is selected from the group consisting of C₅₋₁₄aryl, C₁₋₆ alkyl, C₁₋₆ haloalkyl, and C₅₋₁₄ aralkyl groups;
   R₇-R₁₄ are independently H, C₁₋₆ alkoxy, halo, C₁₋₄alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
   R₁₅ is C₁₋₆ alkyl;
   each M is independently selected from the group consisting of H, an alkali metal, alkaline earth metal, transition metal, ammonium, phosphonium, organic amine salt, and an amino acid salt;
   Z is O or S; and
   n is 0, 1, or 2;
B5. The method of any of B1 to B4, wherein the substrate formulation further comprises:
   a cationic aromatic compound (CAC);
   a background reducing agent; or
   an ether-linked nonionic surfactant that does not contain a carboxylate ester group; or a hydrophilic polymer; or
   a combination thereof.
B6. The method of any of B1 to B5, wherein the substrate formulation comprises
   a) 0.01 mM-50 mM of a chemiluminescent compound of formula I or a salt thereof;
   b) 0.01-200 μM of a cationic aromatic compound (CAC);
   c) 1 μM-10 mM of a background reducing agent; and
   d) 0.05-20 g/L of an ether-linked nonionic surfactant that does not contain a carboxylate ester group; or a hydrophilic polymer.
B7. The method of any of B1 to B6, wherein the measurement of AFP exhibits a coefficient of variation (CV) of less than 7% for concentrations of AFP greater than 10 ng/ml.
B8. The method of any of B1 to B7, wherein the measurement of AFP exhibits a standard deviation (SD) of less than 0.5 ng/mL for concentrations of AFP less than 10 ng/ml.
B9. The method of any of B7 or B8, where the reproducibility conditions of measurement include at least between-site and between-lot variance components.
B10. The method of any of B7 or B8, where the reproducibility conditions of measurement include between-site, between-lot, between-day, between-run, and within-run variance components.

Some exemplary aspects relate to composition comprising any one or more of C1-C20 and related methods comprising any one or more of C21-C35 as follows:
C1. A composition comprising
   an antibody conjugated to alkaline phosphatase (ALP) or an antigen conjugated to ALP;
   a buffer;
   a zinc salt; and
   a magnesium salt;
   wherein
      the concentration of free Zn²⁻ is in a range of 5 μM to 1 mM and the concentration of free Mg²⁺ is in a range of 150 times the concentration of free Zn²⁺ to 3500 times the concentration of free Zn²⁺, when the pH of the composition is in a range of 5.5 to 6.4.
C2. The composition of C1, wherein the enzymatic activity of the ALP varies by less than 25% of peak activity when the composition is stored at 37° C. for up to 3 days.
C3. The composition of C1 or C2, wherein the enzymatic activity of the ALP varies by less than 25% of peak activity when the composition is stored at a temperature in a range of 2° C. to 6° C. for up to 6 months, up to 9 months, or up to 1 year.
C4. A composition comprising
   an antibody conjugated to alkaline phosphatase (ALP) or an antigen conjugated to ALP;
   a buffer;
   a zinc salt; and
   a magnesium salt;
   wherein
      the concentration of free Zn²⁻ is in a range of 5 μM to 1 mM and the concentration of free Mg²⁺ is in a range of 700 times the concentration of free Zn²⁺ to 3500 times the concentration of free Zn²⁺, when the pH of the composition is in a range of 5.5 to 6.4;
   the concentration of free Zn²⁻ is in a range of 1 μM to 5 mM and the concentration of free Mg²⁺ is in a range of 200 times the concentration of free Zn²⁺ to 3500 times the concentration of free Zn²⁺, when the pH of the composition is in a range of 6.5 to 7.4; or
   the concentration of free Zn²⁻ is in a range of 100 nM to 300 UM and the concentration of free Mg²⁺ is in a range of 100 times the concentration of free Zn²⁺ to 1000 times the concentration of free Zn²⁺, when the pH of the composition is in a range of 7.5 to 8.4.
C5. The composition of C4, wherein the enzymatic activity of the ALP varies by less than 10% of peak activity when the composition is stored at 37° C. for up to 3 days.
C6. The composition of C4 or C5, wherein the enzymatic activity of the ALP varies by less than 10% of peak activity when the composition is stored at a temperature in a range of 2° C. to 6° C. for up to 6 months, up to 9 months, or up to 1 year.
C7. The composition of any of C1 to C6, wherein the ALP comprises bovine intestinal ALP.
C8. The composition of any of C1 to C7, wherein the buffer comprises ACES, HEPES, MES, Phosphate, TRIS, ADA, PIPES, MOPSO, BTP, BES, or Bis-TRIS, or combinations thereof.
C9. The composition of any of C1 to C8, wherein the zinc salt comprises ZnCl₂.
C10. The composition of any of C1 to C9, wherein the magnesium salt comprises MgCl₂.
C11. The composition of any of C1 to C10, wherein the composition further comprises albumin.
C12. The composition of C11, wherein the albumin comprises bovine serum albumin, human serum albumin, casein, hydrolyzed casein, or ovalbumin.
C13. The composition of any of C1 to C12, wherein the antibody conjugated to ALP comprises an anti-troponin antibody, an anti-thyroid-stimulating hormone (TSH) antibody, an anti-alpha-fetoprotein (AFP) antibody, an anti-beta human chorionic gonadotropin (βhCG) antibody, an anti-sex hormone-binding globulin (SHBG) antibody, an anti-CA 19-9 antibody, or an anti-creatine kinase-MB (CKMB) antibody.
C14. The composition of C13, wherein the antibody conjugated to ALP comprises an anti-troponin antibody;
the pH of the composition is in a range of 5.5 to 6.4; and the concentration of free Zn²⁺ is in a range of 100 μM to 500 μM and the concentration of free Mg²⁺ is in a range of 150 times the concentration of free Zn²⁺ to 500 times the concentration of free Zn²⁺.
C15. The composition of C13, wherein
   the antibody conjugated to ALP comprises an anti-TSH antibody;
   the pH of the composition is in a range of 5.5 to 6.4; and
   the concentration of free Zn²⁺ is in a range of 5 μM to 100 UM and the concentration of free Mg²⁺ is in a range of 500 times the concentration of free Zn²⁺ to 1500 times the concentration of free Zn²⁺.
C16. The composition of C13, wherein
   the antibody conjugated to ALP comprises an anti-AFP antibody;
   the pH of the composition is in a range of 5.5 to 6.4; and
   the concentration of free Zn²⁺ is in a range of 50 μM to 150 UM and the concentration of free Mg²⁺ is in a range of 150 times the concentration of free Zn²⁺ to 500 times the concentration of free Zn²⁺.
C17. The composition of C13, wherein
   the antibody conjugated to ALP comprises an anti-βhCG antibody;
   the pH of the composition is in a range of 5.5 to 6.4; and
   the concentration of free Zn²⁺ is in a range of 5 μM to 100 M and the concentration of free Mg²⁺ is in a range of 700 times the concentration of free Zn²⁺ to 1500 times the concentration of free Zn²⁺.
C18. The composition of any of C1 to C18, wherein the antigen conjugated to ALP comprises a triiodothyronine (T3) antigen.
C19. The composition of C18, wherein
   the pH of the composition is in a range of 5.5 to 6.4; and
   the concentration of free Zn²⁺ is in a range of 5 μM to 100 UM and the concentration of free Mg²⁺ is in a range of 150 times the concentration of free Zn²⁺ to 500 times the concentration of free Zn²⁺.
C20. The composition of any of C1 to C19, wherein the concentration of free magnesium, free zinc, or a combination thereof, is determined by removing larger molecules from the storage solution by way of size exclusion chromatography, then quantifying free magnesium, free zinc, or a combination thereof, via mass spectroscopy.
C21. A method of using the composition of any of C1 to C20 in an immunoassay.
C22. The method of C21, wherein the immunoassay is performed on an immunoassay analyzer or instrument.
C23. The method of C21 or C22, wherein the method comprises
   contacting an analyte with the composition of any one of C1 to C20 to form a mixture in a vessel,
   incubating the mixture,
   adding a chemiluminescent substrate to the vessel, and
   measuring light generated by the reaction for up to 5 minutes, up to 4 minutes, up to 3 minutes, up to 2 minutes, up to 1 minute, up to 56 seconds, up to 55 seconds, up to 50 seconds, or up to 48 seconds after adding the chemiluminescent substrate to the vessel.
C24. The method of C23, the method further comprising a wash step prior to adding the chemiluminescent substrate to the vessel.
C25. The method of C23 or C24, wherein the chemiluminescent substrate comprises formula I or a salt thereof:

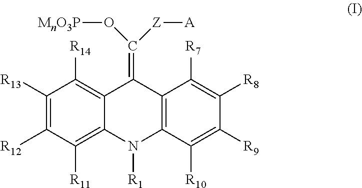

wherein
   A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
   R₁ is selected from the group consisting of C₅₋₁₄aryl, C₁-6 alkyl, C₁₋₆ haloalkyl, and C₅₋₁₄ aralkyl groups;
   R₇-R₁₄ are independently H, C₁₋₆ alkoxy, halo, or C₁₋₄alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
   R₁₅ is C₁₋₆ alkyl;
   each M is independently selected from the group consisting of H, an alkali metal, an alkaline an earth metal, a transition metal, ammonium, phosphonium, an organic amine salt, and an amino acid salt;
   Z is O or S; and
   n is 0, 1, or 2.
C26. The method of C25, wherein a substrate formulation comprising the chemiluminescent substrate further comprises:
   a cationic aromatic compound (CAC);
   a background reducing agent; or
   an ether-linked nonionic surfactant that does not contain a carboxylate ester group; or a hydrophilic polymer; or
   a combination thereof.
C27. The method of any of C23 to C26, wherein the chemiluminescent substrate comprises APS-5.
C28. The method of any of C23 to C27, wherein the method further comprises determining the amount of analyte in the sample.
C29. The method of C28, wherein the amount of analyte is determined from a stored, multi-point calibration curve.
C30. The method of any of C23 to C29, wherein the method comprises contacting a sample comprising the analyte with the composition, wherein the composition comprises an antibody bound to ALP, and further wherein an ALP-antibody-analyte complex is formed.
C31. The method of C30, wherein the method further comprises contacting the ALP-antibody-analyte complex with an anti-analyte antibody bound to a solid phase and washing away sample unbound to the solid phase.
C32. The method of C31, wherein the solid phase comprises paramagnetic particles.
C33. The method of any of C23 to C32, wherein the method comprises contacting a sample comprising the analyte with an antibody specific for the analyte to form a complex comprising the analyte, washing the complex, and contacting the complex with the composition, wherein the composition comprises an antigen bound to ALP.
C34. The method of any of C23 to C33, wherein the method further comprises storing the composition of any one of C1 to C20 for at least 1 month, at least 2 months, at least 3 months, at least 4 months, or at least 5 months prior to using the composition in an immunoassay.
C35. The method of C34, wherein the method further comprises storing the composition of any one of C1 to C20 at a temperature in a range of 2° C. to 6° C. for up to 6 months or up to 1 year prior to using the composition in an immunoassay.

1. A container comprising:
a. at least one reagent comprising particles, wherein a mass of the particles is at least 0.1 μg and up to 100 μg; and
b. a chemiluminescent substrate formulation wherein the chemiluminescent substrate formulation comprises a chemiluminescent compound, or salt thereof, of formula I: 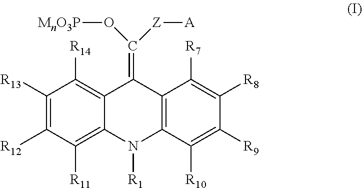
wherein
A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
R₁ is selected from the group consisting of C₅₋₁₄aryl, C₁₋₆ alkyl, C₁₋₆ haloalkyl, and C₅₋₁₄ aralkyl groups;
R₇-R₁₄ are independently H, C₁₋₆ alkoxy, halo, C₁₋₄alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
R₁₅ is C₁₋₆ alkyl;
each M is independently selected from the group consisting of H, an alkali metal, alkaline earth metal, transition metal, ammonium, phosphonium, organic amine salt, and an amino acid salt;
Z is O or S; and
n is 0, 1, or 2.

2. The container of claim 1, wherein the container further comprises:
a. up to 25,000 molecules of analyte, up to 35,000 molecules of analyte or up to 50,000 molecules of analyte;
b. up to 60,000, up to 70,000, up to 80,000, up to 90,000, up to 100,000, or up to 200,000 molecules of enzyme, or
c. a combination thereof.

3. The container of claim 1 or 2, wherein the container further comprises 10,000 to 15,000;
10,000 to 20,000; 10,000 to 25,000; 15,000 to 20,000; or 15,000 to 25,000 molecules of analyte.

4. The container of any of claims 1 to 3, wherein the container comprises buffer.

5. The container of any of claims 1 to 4, wherein the particles comprise paramagnetic particles or paramagnetic microparticles.

6. The container of any of claims 1 to 5, wherein the particles comprise coated paramagnetic particles.

7. The container of any of claims 1 to 6, wherein the particles are coated with antibodies or antigen.

8. The container of any of claims 1 to 7, wherein the at least one reagent comprises
at least 0.1 μg paramagnetic particles, at least 0.5 μg paramagnetic particles, at least 1.0 μg paramagnetic particles, at least 2.5 μg paramagnetic particles, at least 5 μg paramagnetic particles, at least 7.5 μg paramagnetic particles, at least 10 μg paramagnetic particles, at least 15 μg paramagnetic particles, or at least 20 μg paramagnetic particles and/or up to 10 μg paramagnetic particles, up to 15 μg paramagnetic particles, up to 20 μg paramagnetic particles, up to 25 μg paramagnetic particles, up to 30 μg paramagnetic particles, up to 35 μg paramagnetic particles, up to 40 μg paramagnetic particles, up to 45 μg paramagnetic particles, up to 50 μg paramagnetic particles, up to 50 μg paramagnetic particles, up to 75 μg paramagnetic particles, up to 100 μg paramagnetic particles.

9. The container of any of claims 1 to 8, wherein the mass of the chemiluminescent compound is at least 0.1 times, at least 0.5 times, at least the same as, at least 1.5 times, at least two times, at least three times, at least five times, at least ten times, at least twenty-five times, at least fifty times, at least one hundred times, or at least one hundred and fifty times a mass of the at least one reagent.

10. The container of any of claims 1 to 9, wherein the container further comprises an enzyme, an antibody, a capture antibody, an antibody-enzyme conjugate, or an antigen-enzyme conjugate.

11. The container of claim 10, wherein the enzyme is alkaline phosphatase; the antibody-enzyme conjugates comprise ALP; or the antigen-enzyme conjugates comprise ALP.

12. The container of any of the preceding claims, wherein the container is a tube comprising polyethylene.

13. The container of any of the preceding claims, wherein the container is in a luminometer.

14. The container of any of the preceding claims, wherein the chemiluminescent compound is present at a concentration of 0.01 g/L to 50 g/L; 0.1 g/L to 10 g/L; 0.01 g/L to 10 g/L; 0.1 g/L to 30 g/L; or 0.1 g/L to 50 g/L.

15. The container of any of the preceding claims, wherein the chemiluminescent compound is present at a concentration of at least 0.01 g/L, at least 0.05 g/L, at least 0.1 g/L, at least 0.2 g/L, at least 0.5 g/L, at least 1 g/L; or up to 0.5 g/L, up to 1 g/L, up to 2 g/L, up to 5 g/L, up to 7 g/L, or up to 10 g/L.

16. The container of any of the preceding claims, wherein the chemiluminescent compound is present in a volume of 150 μL buffer.

17. The container of any of the preceding claims, wherein the chemiluminescent compound is present in a volume of at least 150 μL and up to 300 μL of buffer.

18. The container of any of the preceding claims, wherein the chemiluminescent compound is present in a volume of up to 200 μL of buffer or up to 500 μL of buffer.

19. The container of any of the preceding claims, wherein the antibody or antigen conjugated to the enzyme comprises an anti-troponin antibody, an anti-thyroid-stimulating hormone (TSH) antibody, an anti-alpha-fetoprotein (AFP) antibody, an anti-beta human chorionic gonadotropin (βhCG) antibody, an anti-sex hormone-binding globulin (SHBG) antibody, an anti-CA 19-9 antibody, or an anti-creatine kinase-MB (CKMB) antibody.

20. The container of any of the preceding claims, wherein the container is situated in an immunoassay analyzer or instrument.

21. The container of claim 20, wherein the immunoassay analyzer or instrument comprises a system that uses disposable tips remove a sample aliquot from a sample, a dispense control system, an image capture device and/or camera unit that may be used to provide feedback on an immunoassay analyzer or instrument performance in real time, a dispenser that heats a wash fluid prior to it being added to the reaction vessel, or a mechanism that is adapted to de-gas a fluid, or a combination thereof.

22. The container of claim 20 or 21, wherein the immunoassay system comprises a luminometer system, the luminometer system, comprising:
a photomultiplier tube configured to sense photons emitted from an assay reaction over a period of time;
an analog circuit configured to provide an assay analog voltage based on the photons emitted from the assay over the period of time, the analog circuit comprising: a current sensing resistor coupled to convert current from the photomultiplier tube to a voltage; an amplifier configured to amplify the voltage; and
a dedicated electrical connection between a terminal of the current sensing resistor and a terminal of the amplifier; a counter circuit configured to provide an assay photon count based on the photons emitted from the assay over the period of time; and
a luminometer controller configured to: in response to the assay analog voltage being greater than a predetermined value, calculating a relative light unit value of the photons emitted from the assay over the period of time based on the assay analog voltage and an optimized linear function.

23. The use of the container of any of claims 1 to 22 in an immunoassay method.

24. An immunoassay analyzer or instrument comprising a container,
wherein the immunoassay analyzer or instrument comprises a system that uses disposable tips remove a sample aliquot from a sample, a dispense control system, an image capture device and/or camera unit that may be used to provide feedback on immunoassay analyzer or instrument performance in real time, a dispenser that heats a wash fluid prior to it being added to the reaction vessel, or a mechanism that is adapted to de-gas a fluid, and/or
wherein the immunoassay system comprises a luminometer system, the luminometer system, comprising:
a photomultiplier tube configured to sense photons emitted from an assay reaction over a period of time;
an analog circuit configured to provide an assay analog voltage based on the photons emitted from the assay over the period of time, the analog circuit comprising: a current sensing resistor coupled to convert current from the photomultiplier tube to a voltage; an amplifier configured to amplify the voltage; and
a dedicated electrical connection between a terminal of the current sensing resistor and a terminal of the amplifier; a counter circuit configured to provide an assay photon count based on the photons emitted from the assay over the period of time; and
a luminometer controller configured to: in response to the assay analog voltage being greater than a predetermined value, calculating a relative light unit value of the photons emitted from the assay over the period of time based on the assay analog voltage and an optimized linear function; and
wherein the container comprises up to 100 μg of paramagnetic microparticles.

25. The immunoassay analyzer or instrument of claim 24, wherein the container further includes an analyte to be detected by the immunoassay analyzer or instrument.

26. An immunoassay analyzer or instrument comprising a container,
wherein the immunoassay analyzer or instrument comprises a system that uses disposable tips remove a sample aliquot from a sample, a dispense control system, an image capture device and/or camera unit that may be used to provide feedback on analyzer or instrument performance in real time, a dispenser that heats a wash fluid prior to it being added to the reaction vessel, or a mechanism that is adapted to de-gas a fluid, and/or
wherein the immunoassay system comprises a luminometer system, the luminometer system, comprising:
a photomultiplier tube configured to sense photons emitted from an assay reaction over a period of time;
an analog circuit configured to provide an assay analog voltage based on the photons emitted from the assay over the period of time, the analog circuit comprising: a current sensing resistor coupled to convert current from the photomultiplier tube to a voltage; an amplifier configured to amplify the voltage; and
a dedicated electrical connection between a terminal of the current sensing resistor and a terminal of the amplifier; a counter circuit configured to provide an assay photon count based on the photons emitted from the assay over the period of time; and
a luminometer controller configured to: in response to the assay analog voltage being greater than a predetermined value, calculating a relative light unit value of the photons emitted from the assay over the period of time based on the assay analog voltage and an optimized linear function; and
wherein the container comprises a sample aliquot with a volume of 2 μl to 50 μl.

27. The immunoassay instrument of any of claims 24 to 26, wherein the container comprises a sample aliquot with a volume of up to 2 μl, up to 5 μl, up to 8 μl, up to 10 μl, up to 15 μl, up to 20 μl, up to 30 μl, up to 40 μl, or up to 50 μl.

28. The immunoassay instrument of claim 26 or 27, wherein the container is a disposable pipette tip.

29. The immunoassay instrument of any of claims 26 to 28, wherein the sample aliquot is an aliquot of serum, an aliquot of plasma, an aliquot of urine, an aliquot of amniotic fluid, an aliquot of whole blood, an aliquot of synovial fluid, an aliquot of cerebrospinal fluid, an aliquot of saliva, an aliquot of seminal fluid, an aliquot of nasal fluid, an aliquot of mucous, or an aliquot of bronchoalveolar lavage.

30. The immunoassay instrument of any of claims 26 to 29 wherein the container is a polyethylene tube.

31. Use of the immunoassay instrument of any of claims 26 to 30 in an immunoassay method.

32. A method comprising:
contacting a sample that is suspected of containing a target analyte with an antibody composition, to form a mixture;
incubating the mixture in a container or vessel; and
adding a substrate composition to the container or vessel to form a reaction mixture;
wherein the antibody composition comprises:
an antibody to the target analyte conjugated to alkaline phosphatase (ALP) or an antigen conjugated to ALP;
a buffer comprising a zinc salt and a magnesium salt;
wherein
the buffer concentration of free Zn²⁺ is in a range of 5 μM to 1 mM and the concentration of free Mg²⁺ is in a range of 150 times the concentration of free Zn²⁺ to 3500 times the concentration of free Zn²⁺, when the pH of the composition is in a range of 5.5 to 6.4; and
wherein the substrate composition comprises:
a chemiluminescent compound, or salt thereof, of formula I: 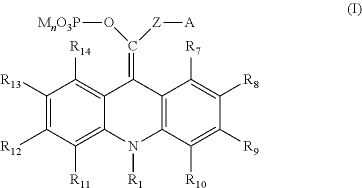
wherein
A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
R₁ is selected from the group consisting of C₅₋₁₄aryl, C₁₋₆ alkyl, C₁₋₆ haloalkyl, and C₅₋₁₄ aralkyl groups;
R₇-R₁₄ are independently H, C₁₋₆ alkoxy, halo, C₁₋₄alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
R₁₅ is C₁₋₆ alkyl;
each M is independently selected from the group consisting of H, an alkali metal, alkaline earth metal, transition metal, ammonium, phosphonium, organic amine salt, and an amino acid salt;
Z is O or S; and
n is 0, 1, or 2.

33. The method of claim 32, wherein the enzymatic activity of the ALP in the ALP-conjugate in the antibody composition varies by less than 25% of peak activity when the composition is stored at 37° C. for up to 3 days.

34. The method of claim 32 or 33, wherein the enzymatic activity of the ALP in the ALP-conjugate in the antibody composition varies by less than 25% of peak activity when the composition is stored at a temperature in a range of 2° C. to 6° C. for up to 6 months, up to 9 months, or up to 1 year.

35. The method of any of claims 32 to 34,
wherein in antibody composition,
(a) the buffer concentration of free Zn²⁺ is in a range of 5 μM to 1 mM and the concentration of free Mg²⁺ is in a range of 700 times the concentration of free Zn²⁺ to 3500 times the concentration of free Zn²⁺, when the pH of the composition is in a range of 5.5 to 6.4;
(b) the buffer concentration of free Zn²⁺ is in a range of 1 μM to 5 mM and the concentration of free Mg²⁺ is in a range of 200 times the concentration of free Zn²⁺ to 3500 times the concentration of free Zn²⁺, when the pH of the composition is in a range of 6.5 to 7.4; or
(c) the buffer concentration of free Zn²⁺ is in a range of 100 nM to 300 UM and the concentration of free Mg²⁺ is in a range of 100 times the concentration of free Zn²⁺ to 1000 times the concentration of free Zn²⁺, when the pH of the composition is in a range of 7.5 to 8.4.

36. The method of claim 35, wherein the enzymatic activity of the ALP in the ALP-conjugate in the antibody composition varies by less than 10% of peak activity when the composition is stored at 37° C. for up to 3 days.

37. The method of claim 35 or 36, wherein the enzymatic activity of the ALP in the antibody composition varies by less than 10% of peak activity when the composition is stored at a temperature in a range of 2° C. to 6° C. for up to 6 months, up to 9 months, or up to 1 year.

38. The method of any of claims 32-37, wherein the ALP in the antibody composition comprises bovine intestinal ALP.

39. The method of any of claims 32-38, wherein the buffer in the antibody composition comprises ACES, HEPES, MES, Phosphate, TRIS, ADA, PIPES, MOPSO, BTP, BES, or Bis-TRIS, or combinations thereof.

40. The method of any of claims 32-39, wherein the zinc salt comprises ZnCl₂.

41. The method of any of claims 32-40, wherein the magnesium salt comprises MgCl₂.

42. The method of any of claims 32-41, wherein the antibody composition further comprises albumin.

43. The method of claim 42, wherein the albumin comprises bovine serum albumin, human serum albumin, casein, hydrolyzed casein, or ovalbumin.

44. The method of any of claims 32-43, wherein the antibody or antigen conjugated to ALP comprises an anti-troponin antibody (cTnI), an anti-thyroid-stimulating hormone (TSH) antibody, an anti-alpha-fetoprotein (AFP) antibody, a triiodothyronine (T3) antigen, an anti-beta human chorionic gonadotropin (βhCG) antibody, an anti-sex hormone-binding globulin (SHBG) antibody, an anti-CA 19-9 antibody, or an anti-creatine kinase-MB (CKMB) antibody.

45. The method of claim 44, wherein
the antibody conjugated to ALP comprises an anti-troponin (cTnI) antibody;
the pH of the antibody composition is in a range of 5.5 to 6.4; and
the concentration of free Zn²⁺ is in a range of 100 μM to 500 UM and the concentration of free Mg²⁺ is in a range of 150 times the concentration of free Zn²⁺ to 500 times the concentration of free Zn²⁺.

46. The method of claim 44, wherein
the antibody conjugated to ALP comprises an anti-TSH antibody;
the pH of the antibody composition is in a range of 5.5 to 6.4; and
the concentration of free Zn²⁺ is in a range of 5 μM to 100 UM and the concentration of free Mg²⁺ is in a range of 500 times the concentration of free Zn²⁺ to 1500 times the concentration of free Zn²⁺.

47. The method of claim 44, wherein
the antibody conjugated to ALP comprises an anti-AFP antibody;
the pH of the antibody composition is in a range of 5.5 to 6.4; and
the concentration of free Zn²⁺ is in a range of 50 μM to 150 UM and the concentration of free Mg²⁺ is in a range of 150 times the concentration of free Zn²⁺ to 500 times the concentration of free Zn²⁺.

48. The method of claim 44, wherein
the antibody conjugated to ALP comprises an anti-βhCG antibody;
the pH of the antibody composition is in a range of 5.5 to 6.4; and
the concentration of free Zn²⁺ is in a range of 5 μM to 100 μM and the concentration of free Mg²⁺ is in a range of 700 times the concentration of free Zn²⁺ to 1500 times the concentration of free Zn²⁺.

49. The method of claim 44, wherein the antigen conjugated to ALP comprises a triiodothyronine (T3) antigen.

50. The method of any of claims 32-49, wherein the concentration of free magnesium, free zinc, or a combination thereof in the antibody composition, is determined by removing larger molecules from the storage solution by way of size exclusion chromatography, then quantifying free magnesium, free zinc, or a combination thereof, via mass spectroscopy.

51. The method of any of claims 32-50, wherein the substrate composition further comprises a cationic aromatic compound (CAC);
a background reducing agent; or
an ether-linked nonionic surfactant that does not contain a carboxylate ester group; or a hydrophilic polymer; or
a combination thereof.

52. The method of claim 51, wherein the substrate composition comprises:
a) 0.01 mM-50 mM of the chemiluminescent compound of formula I or a salt thereof; and
b) 0.01-200 UM of the cationic aromatic compound (CAC);
c) 1 μM to 10 mM of the background reducing agent; or
d) 0.05-20 g/L of the ether-linked nonionic surfactant that does not contain a carboxylate ester group; or a hydrophilic polymer; or
a combination a) with of one or more of b), c), and/or d).

53. The method of any of claims 32-52, wherein substrate composition comprises the compound:

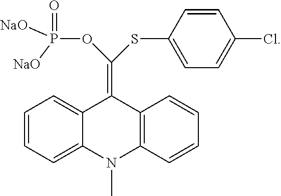

54. The method of any of claims 32-53, wherein the method is an immunoassay method.

55. The method of claim 54, wherein the immunoassay is performed on an immunoassay analyzer or instrument.

56. The method of any of claims 32-55, wherein the method further comprises measuring light generated by the reaction for up to 5 minutes, up to 4 minutes, up to 3 minutes, up to 2 minutes, up to 1 minute, up to 56 seconds, up to 55 seconds, up to 50 seconds, or up to 48 seconds after adding the substrate composition to the vessel.

57. The method of any of claims 32-56, wherein the method further comprises a wash step prior to adding the substrate composition to the vessel.

58. The method of any of claims 32-57, wherein the method further comprises determining the amount of analyte in the sample.

59. The method of claim 58, wherein the amount of analyte is determined from a stored, multi-point calibration curve.

60. The method of any of claims 56 to 59, wherein the light generated by the reaction is measured using a luminometer.

61. The method of any of claims 32-56, wherein the method further comprises at least two wash steps prior to adding the substrate composition to the vessel.

62. The method of any one of claims 32 to 61, wherein the sample suspected of containing the target analyte comprises serum, plasma, urine, amniotic fluid, whole blood, synovial fluid, cerebrospinal fluid, saliva, seminal fluid, nasal fluid, mucous, or bronchoalveolar lavage.

63. The method of any one of claims 32 to 62, wherein the sample suspected of containing the target analyte is a serum sample, a plasma sample, a urine sample, an amniotic fluid sample, or a whole blood sample.

64. The method of any of claims 32-63, wherein the method comprises contacting a sample comprising the analyte with the antibody composition, wherein the antibody composition comprises an antibody bound to ALP, and further wherein an ALP-antibody-analyte complex is formed.

65. The method of claim 64, wherein the method further comprises contacting the ALP-antibody-analyte complex with an anti-analyte antibody bound to a solid phase and washing away sample unbound to the solid phase.

66. The method of claim 65, wherein the solid phase comprises paramagnetic particles, paramagnetic microparticles, or particles.

67. The method of any of claims 57 to 66, wherein the method comprises contacting a sample comprising the analyte with an antibody specific for the analyte to form a complex comprising the analyte, washing the complex, and contacting the complex with the substrate composition, wherein the antibody composition comprises an antigen bound to ALP.

68. The method of any of claims 35-67, wherein the method further comprises storing the antibody composition for at least 1 month, at least 2 months, at least 3 months, at least 4 months, or at least 5 months prior to using the composition in an immunoassay.

69. The method of claim 68, wherein the method further comprises storing the antibody composition at a temperature in a range of 2° C. to 6° C. for up to 6 months or up to 1 year prior to using the composition in an immunoassay.

70. The method of any of claims 35-69, further comprising reproducibility conditions of measurement comprising at least one of between-site, between-lot, between-day, instrument-to-instrument, between-run, and within-run variance components.

71. The method of any of claims 35-70, wherein the target analyte comprises cardiac troponin I (cTnI), alpha-fetoprotein (AFP), thyroid stimulating hormone (TSH), beta human chorionic gonadotropin (βhCG), free thyroxine (T4), creatinine kinase (CK-MB), sex hormone binding globulin (SHBG), or cancer antigen 19-9 (CA 19-9).

72. The method of any of claims 35-71, wherein the target analyte comprises cardiac troponin I (cTnI), alpha-fetoprotein (AFP), thyroid stimulating hormone (TSH), beta human chorionic gonadotropin (βhCG), free thyroxine (T4).

73. The method of any of claims 35-72, wherein the target analyte comprises cardiac troponin I (cTnI).

74. The method of any of claims 35-72, wherein the target analyte comprises alpha-fetoprotein (AFP).

75. The method of any of claims 35-72, wherein the target analyte comprises thyroid stimulating hormone (TSH).

76. The method of any of claims 35-72, wherein the target analyte comprises beta human chorionic gonadotropin (βhCG).

77. The method of any of claims 35-72, wherein the target analyte comprises free thyroxine (T4).

78. An immunoassay method comprising:
a. in a reaction vessel, contacting a sample suspected of containing an analyte with 0.1 μg-100 μg of particles; and
b. subjecting the reaction vessel to further sample processing on an immunoassay instrument.

79. The immunoassay method of claim 78, wherein the immunoassay analyzer or instrument comprises a system that uses disposable tips remove a sample aliquot from a sample, a dispense control system, an image capture device and/or camera unit that may be used to provide feedback on immunoassay analyzer or instrument performance in real time, a dispenser that heats a wash fluid prior to it being added to the reaction vessel, or a mechanism that is adapted to de-gas a fluid, or a combination thereof.

80. The immunoassay method of claim 78 or 79, wherein the immunoassay system comprises a luminometer system, the luminometer system, comprising:
a photomultiplier tube configured to sense photons emitted from an assay reaction over a period of time;
an analog circuit configured to provide an assay analog voltage based on the photons emitted from the assay over the period of time, the analog circuit comprising: a current sensing resistor coupled to convert current from the photomultiplier tube to a voltage; an amplifier configured to amplify the voltage; and
a dedicated electrical connection between a terminal of the current sensing resistor and a terminal of the amplifier; a counter circuit configured to provide an assay photon count based on the photons emitted from the assay over the period of time; and
a luminometer controller configured to: in response to the assay analog voltage being greater than a predetermined value, calculating a relative light unit value of the photons emitted from the assay over the period of time based on the assay analog voltage and an optimized linear function.

81. The immunoassay method of any of claims 78 to 80, the immunoassay method further comprising
adding a chemiluminescent substrate to the reaction vessel, wherein the chemiluminescent substrate is a chemiluminescent compound, or salt thereof, of formula I: 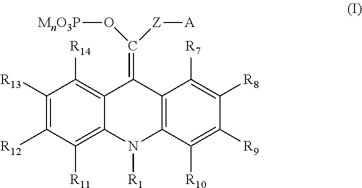
wherein
A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
R₁ is selected from the group consisting of C₅₋₁₄aryl, C₁-6 alkyl, C₁₋₆ haloalkyl, and C₅₋₁₄ aralkyl groups;
R₇-R₁₄ are independently H, C₁₋₆ alkoxy, halo, C₁₋₄alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
R₁₅ is C₁₋₆ alkyl;
each M is independently selected from the group consisting of H, an alkali metal, alkaline earth metal, transition metal, ammonium, phosphonium, organic amine salt, and an amino acid salt;
Z is O or S; and
n is 0, 1, or 2.

82. The immunoassay method of any of claims 78 to 81, wherein the further sample processing comprises:
incubating the reaction vessel;
washing the paramagnetic microparticles;
applying a magnetic field to the paramagnetic microparticles; or
any combination thereof.

83. The immunoassay method of any of claims 78-82, wherein the immunoassay method further comprises allowing the chemiluminescent compound to generate a signal and counting the signal generated by the chemiluminescent compound.

84. The immunoassay method of any of claims 78-83, wherein the immunoassay method further comprises determining the amount of analyte in the sample.

85. The immunoassay method of any of claims 78-84, wherein the amount of the analyte is determined from a stored, multi-point calibration curve.

86. The immunoassay method of any of claims 78-85, wherein the analyte detected by the immunoassay comprises cardiac troponin I (cTnI), alpha-fetoprotein (AFP), thyroid stimulating hormone (TSH), beta human chorionic gonadotropin (βhCG), free thyroxine (T4), creatinine kinase (CK-MB), sex hormone binding globulin (SHBG), or cancer antigen 19-9 (CA 19-9).

87. The immunoassay method of any of claims 78-85, wherein the analyte detected by the immunoassay comprises alpha-fetoprotein (AFP), thyroid stimulating hormone (TSH), beta human chorionic gonadotropin (βhCG), creatinine kinase (CK-MB), sex hormone binding globulin (SHBG), or cancer antigen 19-9 (CA 19-9).

88. The immunoassay method of any of claims 78-87, wherein the sample is a serum sample, a plasma sample, a urine sample, an amniotic fluid sample, a whole blood sample, a synovial fluid sample, a cerebrospinal fluid sample, a saliva sample, a seminal fluid sample, a nasal fluid sample, a mucous sample, or a bronchoalveolar lavage sample.

89. The immunoassay method of any of claims 78-88, wherein the sample is a serum sample, a plasma sample, or an amniotic fluid sample.

90. The immunoassay method of any of claims 78-89, wherein the analyte detected by the immunoassay comprises AFP and a Limit of Blank (LoB) is 0.08-0.16 ng/ml, a Limit of Detection (LoD) is 0.22-0.23 ng/mL, and/or a Limit of Quantitation (LoQ) is 0.13-0.20.

91. The immunoassay method of any of claims 78-89, wherein the analyte detected by the immunoassay comprises TSH and a Limit of Blank (LoB) is 0.001-0.002 uIU/mL, a Limit of Detection (LoD) is 0.002-0.003 uIU/mL, and/or a Limit of Quantitation (LoQ) is 0.0008-0.001 uIU/mL.

92. The immunoassay method of any of claims 78-89, wherein the analyte detected by the immunoassay comprises hCG and a Limit of Blank (LoB) is 0.0-0.1 mIU/mL, a Limit of Detection (LoD) is 0.2 mIU/mL, and/or a Limit of Quantitation (LoQ) is 0.1-0.2 mIU/mL.

93. The immunoassay method of any of claims 78-89, wherein the analyte detected by the immunoassay comprises free T4 and a Limit of Blank (LoB) is 0.18 ng/mL.

94. The immunoassay method of any of claims 78-93, wherein the particles are paramagnetic particles, microparticles, or paramagnetic microparticles.

95. The immunoassay method of any of claims 78-94, wherein the particles are coated particles.

96. The immunoassay method of any of claims 78-95, wherein the particles are coated with antibodies.

97. The immunoassay method of claim 96, wherein the particles are coated with monoclonal antibodies, target-specific antibodies, or target-specific monoclonal antibodies.

98. The immunoassay method of any of claims 78-97, wherein the particles comprise at least 0.1 μg paramagnetic particles, at least 0.5 μg paramagnetic particles, at least 1.0 μg paramagnetic particles, at least 2.5 μg paramagnetic particles, at least 5 μg paramagnetic particles, at least 7.5 μg paramagnetic particles, at least 10 μg paramagnetic particles, at least 15 μg paramagnetic particles, or at least 20 μg paramagnetic particles and/or up to 10 μg paramagnetic particles, up to 15 μg paramagnetic particles, up to 20 μg paramagnetic particles, up to 25 μg paramagnetic particles, up to 30 μg paramagnetic particles, up to 35 μg paramagnetic particles, up to 40 μg paramagnetic particles, up to 45 μg paramagnetic particles, up to 50 μg paramagnetic particles, up to 50 μg paramagnetic particles, up to 75 μg paramagnetic particles, up to 100 μg paramagnetic particles.

99. An immunoassay method to detect alpha-fetoprotein (AFP) in a sample, wherein the immunoassay method comprises:
contacting a sample that is suspected of containing AFP with an anti-AFP antibody conjugated to an enzyme, to form a mixture, wherein the sample is a blood sample, a plasma sample, or an amniotic fluid sample;
incubating the mixture in a container or vessel;
adding a chemiluminescent substrate to the container or vessel to form a reaction mixture;
incubating the reaction mixture;
measuring light generated by the reaction mixture; and
detecting a concentration of AFP in the sample from the light generated by the reaction mixture;
wherein a Limit of Blank (LoB) of the immunoassay method is 0.08-0.16 ng/ml, a Limit of Detection (LoD) of the immunoassay method is 0.22-0.23 ng/mL, and/or a Limit of Quantitation (LoQ) of the immunoassay method is 0.13-0.20.

100. An immunoassay method to detect thyroid stimulating hormone (TSH) in a sample, wherein the immunoassay method comprises:
contacting a sample that is suspected of containing TSH with an anti-TSH antibody conjugated to an enzyme, to form a mixture, wherein the sample is a plasma sample, a urine sample, an amniotic fluid sample, a whole blood sample, a synovial fluid sample, a cerebrospinal fluid sample, a saliva sample, a seminal fluid sample, a nasal fluid sample, a mucous sample, or a bronchoalveolar lavage sample;
incubating the mixture in a container or vessel;
adding a chemiluminescent substrate to the container or vessel to form a reaction mixture;
incubating the reaction mixture;
measuring light generated by the reaction mixture; and
detecting a concentration of TSH in the sample from the light generated by the reaction mixture;
wherein a Limit of Blank (LoB) of the immunoassay method is 0.001-0.002 uIU/mL, a Limit of Detection (LoD) of the immunoassay method is 0.002-0.003 uIU/mL, and/or a Limit of Quantitation (LoQ) of the immunoassay method is 0.0008-0.001 uIU/mL.

101. An immunoassay method to detect beta human chorionic gonadotropin (βhCG) in a sample, wherein the immunoassay method comprises:
contacting a sample that is suspected of containing βhCG with an anti-βhCG antibody conjugated to an enzyme, to form a mixture, wherein the sample is a plasma sample, a urine sample, an amniotic fluid sample, a whole blood sample, a synovial fluid sample, a cerebrospinal fluid sample, a saliva sample, a seminal fluid sample, a nasal fluid sample, a mucous sample, or a bronchoalveolar lavage sample;
incubating the mixture in a container or vessel;
adding a chemiluminescent substrate to the container or vessel to form a reaction mixture;
incubating the reaction mixture;
measuring light generated by the reaction mixture; and
detecting a concentration of βhCG in the sample from the light generated by the reaction mixture;
wherein a Limit of Blank (LoB) of the immunoassay method is 0.0-0.1 mIU/mL, a Limit of Detection (LoD) of the immunoassay method is 0.2 mIU/mL, and/or a Limit of Quantitation (LoQ) of the immunoassay method is 0.1-0.2 mIU/mL.

102. An immunoassay method to detect free thyroxine 4 (T4) in a sample, wherein the immunoassay method comprises:
contacting a sample that is suspected of containing T4 with an anti-T4 antibody conjugated to an enzyme, to form a mixture, wherein the sample is a plasma sample, a urine sample, an amniotic fluid sample, a whole blood sample, a synovial fluid sample, a cerebrospinal fluid sample, a saliva sample, a seminal fluid sample, a nasal fluid sample, a mucous sample, or a bronchoalveolar lavage sample;
incubating the mixture in a container or vessel;
adding a chemiluminescent substrate to the container or vessel to form a reaction mixture, wherein the chemiluminescent substrate is a substrate for the enzyme;
incubating the reaction mixture;
measuring light generated by the reaction mixture; and
detecting a concentration of T4 in the sample from the light generated by the reaction mixture;
wherein a Limit of Blank (LoB) of the immunoassay method is less than 0.18 ng/mL.

103. The immunoassay method of any of claims 99-102, wherein the chemiluminescent substrate comprises a chemiluminescent compound, or salt thereof, of formula I: 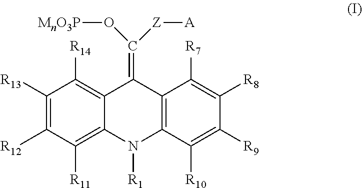
A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
R₁ is selected from the group consisting of C₅₋₁₄aryl, C₁-6 alkyl, C₁₋₆ haloalkyl, and C₅₋₁₄ aralkyl groups;
R₇-R₁₄ are independently H, C₁₋₆ alkoxy, halo, C₁₋₄alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
R₁₅ is C₁₋₆ alkyl;
each Mis independently selected from the group consisting of H, an alkali metal, alkaline earth metal, transition metal, ammonium, phosphonium, organic amine salt, and an amino acid salt;
Z is O or S; and
n is 0, 1, or 2.

104. The immunoassay method of claim 103, wherein the immunoassay method is conducted using an immunoassay analyzer or instrument.

105. The immunoassay method of claim 104, wherein the immunoassay analyzer or instrument comprises a system that uses disposable tips remove a sample aliquot from a sample, a dispense control system, an image capture device and/or camera unit that may be used to provide feedback on immunoassay analyzer or instrument performance in real time, a dispenser that heats a wash fluid prior to it being added to the reaction vessel, or a mechanism that is adapted to de-gas a fluid, or a combination thereof.

106. The immunoassay method of any of claims 104-105, wherein the immunoassay method is conducted using an immunoassay system that comprises a luminometer system, the luminometer system, comprising:
a photomultiplier tube configured to sense photons emitted from an assay reaction over a period of time;
an analog circuit configured to provide an assay analog voltage based on the photons emitted from the assay over the period of time, the analog circuit comprising: a current sensing resistor coupled to convert current from the photomultiplier tube to a voltage; an amplifier configured to amplify the voltage; and
a dedicated electrical connection between a terminal of the current sensing resistor and a terminal of the amplifier; a counter circuit configured to provide an assay photon count based on the photons emitted from the assay over the period of time; and
a luminometer controller configured to: in response to the assay analog voltage being greater than a predetermined value, calculating a relative light unit value of the photons emitted from the assay over the period of time based on the assay analog voltage and an optimized linear function.
